SEQUENCE LISTING (344 sequences)

SEQ ID NO: 1 - DNA, 85 bp, synthetic construct
  source 1..85: mol_type = other DNA
tatgctatgaggatccccacaatcagaaggtacagcgattttcttcccatcaggtctcgatctcgtacaaaacgactatgaccat

SEQ ID NO: 2 - DNA, 75 bp, synthetic construct
  source 1..75: mol_type = other DNA
cgaaagccatgacctccgatcactctcattctccatggctcctcaacgttatctgggagaggcgtataaatataa

SEQ ID NO: 3 - DNA, 85 bp, synthetic construct
  source 1..85: mol_type = other DNA
ttctggccaccagagagctgagttcctttgtctcctactttagtgctatacttacggttttgacattccggaaatttggatcgaa

SEQ ID NO: 4 - DNA, 75 bp, synthetic construct
  source 1..75: mol_type = other DNA
cgaaagccatgacctccgatcactcgcaaaatatgaggctgtctaacaagggcacgagctatggcaatgcgttgt

SEQ ID NO: 5 - DNA, 85 bp, synthetic construct
  source 1..85: mol_type = other DNA
gaagtctggctgccaaaactcctccgtgggaagcaccaggaaaccacttgcagaagatcaaaaaacgatccctgtccatcaatac

SEQ ID NO: 6 - DNA, 75 bp, synthetic construct
  source 1..75: mol_type = other DNA
cgaaagccatgacctccgatcactccagtaggctgaatttggtcactaggatcttctatacaaccaattcctcca

SEQ ID NO: 7 - DNA, 85 bp, synthetic construct
  source 1..85: mol_type = other DNA
cagcacagttaccctgtaaggcactttacaggtgcaaaatcttaaggtgcctagcccgcgcaactcgtcctattaaaagtcggaa

SEQ ID NO: 8 - DNA, 75 bp, synthetic construct
  source 1..75: mol_type = other DNA
cgaaagccatgacctccgatcactccttaggcgtgtcattcaccacttggggatcatttcctcatctaaagcatt

SEQ ID NO: 9 - DNA, 85 bp, synthetic construct
  source 1..85: mol_type = other DNA
tgcactgagaggatcgtccaggagatagatgtcagcatcttgatacactgcctgcattctcatggaaatgcaatggattcattcc

SEQ ID NO: 10 - DNA, 75 bp, synthetic construct
  source 1..75: mol_type = other DNA
cgaaagccatgacctccgatcactcatttgacaaatacacagttcgaacaagtgtctgctaacttccgcatctac

SEQ ID NO: 11 - DNA, 85 bp, synthetic construct
  source 1..85: mol_type = other DNA
agcaggaaagtgttttagaagcatttgcggtggacaatggaaggcccggcctacggttaccgtctttataagtgaacaaaaccgg

SEQ ID NO: 12 - DNA, 75 bp, synthetic construct
  source 1..75: mol_type = other DNA
cgaaagccatgacctccgatcactcaaggcataattccacaggacattcacagttgtgtgctagagacagagagg

SEQ ID NO: 13 - DNA, 81 bp, synthetic construct
  source 1..81: mol_type = other DNA
atcggcaaaggcgaggctctgtgctcgcggggcggacgcggtctcgcatttggaatgatgtgtactgggaataagacgacg

SEQ ID NO: 14 - DNA, 74 bp, synthetic construct
  source 1..74: mol_type = other DNA
cgaaagccatgacctccgatcactccgatatcatcatccatggtgagctggcggcgggtgtggacgggcggcgg

SEQ ID NO: 15 - DNA, 85 bp, synthetic construct
  source 1..85: mol_type = other DNA
ttggaaactggacagaacaatctggaactctcatctcctgttgctcctctcagagttgtcaagacgttggcgtgatgggatgaac

SEQ ID NO: 16 - DNA, 75 bp, synthetic construct
  source 1..75: mol_type = other DNA
cgaaagccatgacctccgatcactcgtagcacagttgctaatagctgaaaaccggcacgaaaactttactgccct

SEQ ID NO: 17 - DNA, 73 bp, synthetic construct
  source 1..73: mol_type = other DNA
ggcgcggggcaggggtccgaaggcctgcacgcatttgcccttggtgaaaagtctcttgttattttgtgctcgc

SEQ ID NO: 18 - DNA, 71 bp, synthetic construct
  source 1..71: mol_type = other DNA
cgaaagccatgacctccgatcactctgctgtcctgacagggcgaggtcctctccaagcgcacgtgatgctc

SEQ ID NO: 19 - DNA, 85 bp, synthetic construct
  source 1..85: mol_type = other DNA
agaaggagggccgtgcccaaaggcacaaagcaattacggtagaatcccagcacaagaatccctgctagctgaaggagggtcaaac

SEQ ID NO: 20 - DNA, 71 bp, synthetic construct
  source 1..71: mol_type = other DNA
cgaaagccatgacctccgatcactctcttccccctccaagcaccattgggagcaaggtgcatgttaggaaa

SEQ ID NO: 21 - DNA, 85 bp, synthetic construct
  source 1..85: mol_type = other DNA
aaaaaggtctgagttcaagtgttggctccaccatatactacctgtgagaccagataaggttgttattgtggaggatgttactaca

SEQ ID NO: 22 - DNA, 75 bp, synthetic construct
  source 1..75: mol_type = other DNA
cgaaagccatgacctccgatcactctcccaggagcctaggatttgtctcagtggaaaaatcatgggattttgtgt

SEQ ID NO: 23 - DNA, 73 bp, synthetic construct
  source 1..73: mol_type = other DNA
tttcctctccttatgcacagatggacaggctgccttccctttcgttgggacgcttgaagcgcaagtagaaaac

SEQ ID NO: 24 - DNA, 66 bp, synthetic construct
  source 1..66: mol_type = other DNA
cgaaagccatgacctccgatcactcctcctccatgtgaagcccctgaaacatacacaccctggaac

SEQ ID NO: 25 - DNA, 85 bp, synthetic construct
  source 1..85: mol_type = other DNA
aatcaggtttaatagtggtccattatctcactgccacagacacacaactgccagcagacctgcaatatcaaagttataagcgcgt

SEQ ID NO: 26 - DNA, 75 bp, synthetic construct
  source 1..75: mol_type = other DNA
cgaaagccatgacctccgatcactctgacgactctctgggaatctcacacatcactctttcctagcaccgaagag

SEQ ID NO: 27 - DNA, 85 bp, synthetic construct
  source 1..85: mol_type = other DNA
ggtctacagttgattgtagcattgtggtccagtagcaccagaagacattccgaggaaacaagtgggaggtttgtgaagaggttta

SEQ ID NO: 28 - DNA, 75 bp, synthetic construct
  source 1..75: mol_type = other DNA
cgaaagccatgacctccgatcactcaatccacattggccatttcacaagccacgtgaagaggggttttcccattg

SEQ ID NO: 29 - DNA, 85 bp, synthetic construct
  source 1..85: mol_type = other DNA
gagcaagccaggtagctgaagtcttgtcaaaagagacaccactctgattccaatatgccaattgaggcgagggaactgggattgt

SEQ ID NO: 30 - DNA, 75 bp, synthetic construct
  source 1..75: mol_type = other DNA
cgaaagccatgacctccgatcactcctggttagcctgaaacactgccctgttacaaagacctgcaattctggaca

SEQ ID NO: 31 - DNA, 80 bp, synthetic construct
  source 1..80: mol_type = other DNA
tccatgttcacccccatggctgcaaagacgatggcgaagttgtcgcttacagatcgtgtgctcatgacttccacagacgt

SEQ ID NO: 32 - DNA, 72 bp, synthetic construct
  source 1..72: mol_type = other DNA
cgaaagccatgacctccgatcactcccatggttccattctgctcaaagtcagacttgaagaatctggctgtc

SEQ ID NO: 33 - DNA, 85 bp, synthetic construct
  source 1..85: mol_type = other DNA
gggctcacgtcaggaaggcgcaggaattcagaaaactctaaagcttggaacactcactaattgtccgtgcgacgacaaattaccg

SEQ ID NO: 34 - DNA, 75 bp, synthetic construct
  source 1..75: mol_type = other DNA
cgaaagccatgacctccgatcactccacagagagaccaacaggacgcatggcagaactgcatagaagatcgctca

SEQ ID NO: 35 - DNA, 70 bp, synthetic construct
  source 1..70: mol_type = other DNA
gtgacataggtgcgacggccccagggctccgcaaaccaatttggttttactcccctcgattatgcggagt

SEQ ID NO: 36 - DNA, 60 bp, synthetic construct
  source 1..60: mol_type = other DNA
cgaaagccatgacctccgatcactcgggtaggtgccacgaacaccatcagggcaatccag

SEQ ID NO: 37 - DNA, 85 bp, synthetic construct
  source 1..85: mol_type = other DNA
agacaagtctgaatgctccactttttcaattctctctccattcttcagtaccacgcgatgacgttcgtcaagagtcgcataatct

SEQ ID NO: 38 - DNA, 75 bp, synthetic construct
  source 1..75: mol_type = other DNA
cgaaagccatgacctccgatcactcggggtgaattcagtgtagtacaagagatagaaagaccagtccttgctgaa

SEQ ID NO: 39 - DNA, 73 bp, synthetic construct
  source 1..73: mol_type = other DNA
tcttcctcctcctccccagactgaagccggtaaagatcctagctgttatggctattgctgaaacagcaaaatt

SEQ ID NO: 40 - DNA, 62 bp, synthetic construct
  source 1..62: mol_type = other DNA
cgaaagccatgacctccgatcactcgcgctcaggatactcaagaccagtgctgtggatctgc

SEQ ID NO: 41 - DNA, 85 bp, synthetic construct
  source 1..85: mol_type = other DNA
actcctctgttaggatgaaaacgaggtgttaccactggccttttctatagccgagatacgtcttaccgcacataggtaacctatt

SEQ ID NO: 42 - DNA, 75 bp, synthetic construct
  source 1..75: mol_type = other DNA
cgaaagccatgacctccgatcactcatgagtaatgatcaagaaaggaacgatccatgctggtgccattcgcacct

SEQ ID NO: 43 - DNA, 85 bp, synthetic construct
  source 1..85: mol_type = other DNA
ctgaaatcgctgcaactcaaggcttcaagggagagcaagatccctttcaacagtttcatgctaatcaaagaagagctaatccgac

SEQ ID NO: 44 - DNA, 75 bp, synthetic construct
  source 1..75: mol_type = other DNA
cgaaagccatgacctccgatcactcctgtagttcagcctaaacaccgagacaggcttacacagggagagaagaca

SEQ ID NO: 45 - DNA, 81 bp, synthetic construct
  source 1..81: mol_type = other DNA
ccccagcacagagaataagagcaagatccgttcggtacagcttccccttgacgtagattgctatcaggttacgatgactgc

SEQ ID NO: 46 - DNA, 75 bp, synthetic construct
  source 1..75: mol_type = other DNA
cgaaagccatgacctccgatcactctagatacagtgtatcactagcaagtggttgcggccaccaactctagttat

SEQ ID NO: 47 - DNA, 85 bp, synthetic construct
  source 1..85: mol_type = other DNA
ctcttctgtcttctgaggccaggagaggagttgggaaatgtgagcaattccataaaattggttttgcctttcagcaattcaactt

SEQ ID NO: 48 - DNA, 75 bp, synthetic construct
  source 1..75: mol_type = other DNA
cgaaagccatgacctccgatcactcatgggcctttttcattttctgggcagctgatgggaggaataacccagtct

SEQ ID NO: 49 - DNA, 81 bp, synthetic construct
  source 1..81: mol_type = other DNA
acatctgggcattgtggctgaccaccacggggattatgtgttcacgctcgtaaccccgggcactagattatggtgaatggt

SEQ ID NO: 50 - DNA, 66 bp, synthetic construct
  source 1..66: mol_type = other DNA
cgaaagccatgacctccgatcactccacgttgcagcgacacacgatcactcgaaccaggagctgcc

SEQ ID NO: 51 - DNA, 71 bp, synthetic construct
  source 1..71: mol_type = other DNA
tatgatgtctaccctgttctcaggaacttcaccatactatttctgttcacggatgaaggcctatatcaatg

SEQ ID NO: 52 - DNA, 61 bp, synthetic construct
  source 1..61: mol_type = other DNA
cgaaagccatgacctccgatcactcgggttgatccttatcggtcacagttagattagctac

SEQ ID NO: 53 - DNA, 85 bp, synthetic construct
  source 1..85: mol_type = other DNA
agttggctgacctgacatggagtgtagtaaaacaccaggacagcggaagccggctatgattcaccaccgtgtaggagttatgcgc

SEQ ID NO: 54 - DNA, 75 bp, synthetic construct
  source 1..75: mol_type = other DNA
cgaaagccatgacctccgatcactcttaaagcacagtcctttatcccattagcctgtgaaatgtacagttagggc

SEQ ID NO: 55 - DNA, 85 bp, synthetic construct
  source 1..85: mol_type = other DNA
agagagtctccccaaagaggcgcccgatagcagctccatagacaaagatgcggatgtgtcgctaacctcctggtcctataaagca

SEQ ID NO: 56 - DNA, 75 bp, synthetic construct
  source 1..75: mol_type = other DNA
cgaaagccatgacctccgatcactcgatgggattggtgatccctccagccacgatgccctcagggaagataaaag

SEQ ID NO: 57 - DNA, 85 bp, synthetic construct
  source 1..85: mol_type = other DNA
aactcaagaggcagcttgccagcgagctcttttagacataagcatttttacagaaatgtcactcccatggtggctgatatagaaa

SEQ ID NO: 58 - DNA, 75 bp, synthetic construct
  source 1..75: mol_type = other DNA
cgaaagccatgacctccgatcactcatgggagggccttgatgggatcattttgtgaacagttcgcttttataaca

SEQ ID NO: 59 - DNA, 85 bp, synthetic construct
  source 1..85: mol_type = other DNA
aaaaattgcccagaagtaaactttgtgctggaacccctgcctccattgaccatgtcgaaccttggataggagcgaccgattacgt

SEQ ID NO: 60 - DNA, 75 bp, synthetic construct
  source 1..75: mol_type = other DNA
cgaaagccatgacctccgatcactcccctgtgctctaaagacatttcctcgcattcacattatttccttggatac

SEQ ID NO: 61 - DNA, 85 bp, synthetic construct
  source 1..85: mol_type = other DNA
gccctgcggcacaagggattgacacgcgttccccaaatccgatgtttctgctctgtgaactgtcatcggtccgatcaattagtct

SEQ ID NO: 62 - DNA, 75 bp, synthetic construct
  source 1..75: mol_type = other DNA
cgaaagccatgacctccgatcactcgtctttcagcaacacagttacacaaggaacagaacagtctctcccgccca

SEQ ID NO: 63 - DNA, 76 bp, synthetic construct
  source 1..76: mol_type = other DNA
cctctctttccttcttcaccactgggaccaggaggaccttgcctatgcatcatgtgcctcactaggacatcatgct

SEQ ID NO: 64 - DNA, 60 bp, synthetic construct
  source 1..60: mol_type = other DNA
cgaaagccatgacctccgatcactcctggagggccggcagatccagcttccccattaggg

SEQ ID NO: 65 - DNA, 85 bp, synthetic construct
  source 1..85: mol_type = other DNA
gaccccttggtcacccttgtcaccttttggtccttgaaaacttaagcccacgggaaaaccattgatcatgtattctttgtcgcat

SEQ ID NO: 66 - DNA, 75 bp, synthetic construct
  source 1..75: mol_type = other DNA
cgaaagccatgacctccgatcactctctcctttttcttgaacttgagcttgtcctggtactcctggaggcccact

SEQ ID NO: 67 - DNA, 85 bp, synthetic construct
  source 1..85: mol_type = other DNA
ctcagtccaggaaagcccatgattccttcttctcccttcaaggaaatgccccctatgttgttgttgaggagatttatgttgtggg

SEQ ID NO: 68 - DNA, 71 bp, synthetic construct
  source 1..71: mol_type = other DNA
cgaaagccatgacctccgatcactccttctgtcctggtgatcctttttcaccactcaagccagggtaaccc

SEQ ID NO: 69 - DNA, 79 bp, synthetic construct
  source 1..79: mol_type = other DNA
ctttgacccagtggtcccatttcaccttgatgacctggaattccctctaatacatcgtgatacgggcgatataatgctc

SEQ ID NO: 70 - DNA, 72 bp, synthetic construct
  source 1..72: mol_type = other DNA
cgaaagccatgacctccgatcactccatcttcacctggtggccctaaaattcccggatttcctggatatcct

SEQ ID NO: 71 - DNA, 76 bp, synthetic construct
  source 1..76: mol_type = other DNA
agttcctttctgacctttcaatccatgcaagccgttcaggccgaatttgtccgctgggtaatggtggaattaaaga

SEQ ID NO: 72 - DNA, 67 bp, synthetic construct
  source 1..67: mol_type = other DNA
cgaaagccatgacctccgatcactctggaccaggtggccccacatcatgcaaacctgaagcaccttt

SEQ ID NO: 73 - DNA, 85 bp, synthetic construct
  source 1..85: mol_type = other DNA
agaagcacagggaaaagaattgttaattctgcgtgtgtgtttttgtttctctttacttgttgtcaccccagagatgttctcaatt

SEQ ID NO: 74 - DNA, 75 bp, synthetic construct
  source 1..75: mol_type = other DNA
cgaaagccatgacctccgatcactcgttgtttcccagttctgtacaagcagaggccacaggaggattcttacata

SEQ ID NO: 75 - DNA, 85 bp, synthetic construct
  source 1..85: mol_type = other DNA
acccttttgaccctcaattccttggaatccattaagcccaggaaatcccactcgtcaatgatctacgatcctcgctaagtattca

SEQ ID NO: 76 - DNA, 75 bp, synthetic construct
  source 1..75: mol_type = other DNA
cgaaagccatgacctccgatcactcacagcaccatctatatcgatgaaaacatctgggcctggcaggccaatgtc

SEQ ID NO: 77 - DNA, 83 bp, synthetic construct
  source 1..83: mol_type = other DNA
ctggtgcagaccacgtcatcggcgccaaacgtcaggataagttcatcgcttcagttaaaggctatcttgctccgctcgttctc

SEQ ID NO: 78 - DNA, 75 bp, synthetic construct
  source 1..75: mol_type = other DNA
cgaaagccatgacctccgatcactccttcctgaaagtaggagcaagccagctgccttcactctcggacataaatt

SEQ ID NO: 79 - DNA, 85 bp, synthetic construct
  source 1..85: mol_type = other DNA
gagacgaatctcagcgtcagggcgctgctgaaggatgtgataggatggggccaccgttttgccagttccaccagtaactgtaagg

SEQ ID NO: 80 - DNA, 75 bp, synthetic construct
  source 1..75: mol_type = other DNA
cgaaagccatgacctccgatcactcctgatgcggtgaccgtggtgtggaaacccccagcactgatgctagaatca

SEQ ID NO: 81 - DNA, 85 bp, synthetic construct
  source 1..85: mol_type = other DNA
gcttcgtttgcgtcgttcgtggctggaaggaacagtggtggagaatactaccggcacaagcagacaaaatcaacatggtcattta

SEQ ID NO: 82 - DNA, 75 bp, synthetic construct
  source 1..75: mol_type = other DNA
cgaaagccatgacctccgatcactcgtgtaatagagagaggatagagagctcctgttcggagctgggggaacttg

SEQ ID NO: 83 - DNA, 85 bp, synthetic construct
  source 1..85: mol_type = other DNA
cccccacccccatcccgcaaagaaaaagtaacagtcgcagaagaggcaggcagttcttaattccattggctctctcaatgggtcg

SEQ ID NO: 84 - DNA, 62 bp, synthetic construct
  source 1..62: mol_type = other DNA
cgaaagccatgacctccgatcactcccgggacatgggcctggacaaccacctggagagggaa

SEQ ID NO: 85 - DNA, 85 bp, synthetic construct
  source 1..85: mol_type = other DNA
agtctcaaaggagattctcacacagtatgtgtgccaccaagctctcgagacagtgcgttttataaatacagggatgctgtttttc

SEQ ID NO: 86 - DNA, 63 bp, synthetic construct
  source 1..63: mol_type = other DNA
cgaaagccatgacctccgatcactccagagtgtggaattctgtgcctggaccctgttccatga

SEQ ID NO: 87 - DNA, 71 bp, synthetic construct
  source 1..71: mol_type = other DNA
tattttctctgatgtctccaacagagccttcactcccgatattttgctgatgtaactgttactgaccttgc

SEQ ID NO: 88 - DNA, 61 bp, synthetic construct
  source 1..61: mol_type = other DNA
cgaaagccatgacctccgatcactccagccgcttatcaagcacatccaatgacacagctgt

SEQ ID NO: 89 - DNA, 81 bp, synthetic construct
  source 1..81: mol_type = other DNA
gagcccaagcactgagcagggaggtgaacgccagtccagaaagaagctgtgtccgtctatacgcatactggtccacatata

SEQ ID NO: 90 - DNA, 75 bp, synthetic construct
  source 1..75: mol_type = other DNA
cgaaagccatgacctccgatcactcacatatttatatagcacataaattagggagtgctctgacccctgcccgtg

SEQ ID NO: 91 - DNA, 85 bp, synthetic construct
  source 1..85: mol_type = other DNA
agaagtttgcatcagggttttaagcctcaggatgttaggaaagggaatgtcaccgtgtggacggcaactcagagataacgcatat

SEQ ID NO: 92 - DNA, 75 bp, synthetic construct
  source 1..75: mol_type = other DNA
cgaaagccatgacctccgatcactcgcattgcagtgaatggttgaactcggccaatttctccaaccactgaaagg

SEQ ID NO: 93 - DNA, 85 bp, synthetic construct
  source 1..85: mol_type = other DNA
gcaaagcacaagtgacacatggtccctctctaacacctcagcacaccaaccccgaatgtataatgctgacgttcttgcttttggc

SEQ ID NO: 94 - DNA, 75 bp, synthetic construct
  source 1..75: mol_type = other DNA
cgaaagccatgacctccgatcactctatgcacagttcacggctcatatgcaaagtggaagacacgtgggacaaga

SEQ ID NO: 95 - DNA, 85 bp, synthetic construct
  source 1..85: mol_type = other DNA
ttccatcaggttgtctggtgcagaaagcacaaagcaggcaacaaggcgggcctcaagacctaagcgacagcgtgaccttgtttca

SEQ ID NO: 96 - DNA, 75 bp, synthetic construct
  source 1..75: mol_type = other DNA
cgaaagccatgacctccgatcactctgtccaggctgccctgcacacttcaaaaatgtacaactcaggtgcaaatg

SEQ ID NO: 97 - DNA, 85 bp, synthetic construct
  source 1..85: mol_type = other DNA
attgctattcacagtctcatctatatataattggtgcttttccagttctgcgctttagaatgcttaaagatggcagagttggagg

SEQ ID NO: 98 - DNA, 75 bp, synthetic construct
  source 1..75: mol_type = other DNA
cgaaagccatgacctccgatcactccttctcaggttttccaggattgaacgaagcacacgaaggttagttgggat

SEQ ID NO: 99 - DNA, 73 bp, synthetic construct
  source 1..73: mol_type = other DNA
ccagcctgcagagcagcgcacagctcatcttgcttgagctgatgtacagggtgaaggaactgtaacggcagct

SEQ ID NO: 100 - DNA, 75 bp, synthetic construct
  source 1..75: mol_type = other DNA
cgaaagccatgacctccgatcactcaacagcaaacaatatcaacaaccggaacccagggctcccccagcacctcc

SEQ ID NO: 101 - DNA, 70 bp, synthetic construct
  source 1..70: mol_type = other DNA
tcgatgtggtctgcacagaggtgtgcctctcacacctcccctttcccaagtaaatgtacgggaattatcg

SEQ ID NO: 102 - DNA, 75 bp, synthetic construct
  source 1..75: mol_type = other DNA
cgaaagccatgacctccgatcactcctgcggttggtaaacagctgcacgaacatcggtgaaggggccagatccgc

SEQ ID NO: 103 - DNA, 85 bp, synthetic construct
  source 1..85: mol_type = other DNA
gcgcctggaggagcgaacaaaacaccgaaccaccaagacgagaaaaggagcacaaactcactactaccaacaacctcaccaaaaa

SEQ ID NO: 104 - DNA, 74 bp, synthetic construct
  source 1..74: mol_type = other DNA
cgaaagccatgacctccgatcactctccgagaattcgcagcggcgaaaatgggcgcgcgaggtcgagaggggcc

SEQ ID NO: 105 - DNA, 80 bp, synthetic construct
  source 1..80: mol_type = other DNA
aagcgttgaaaggcggacggaggctggaggaggcggccaggagcgcctgccaatgcactcgatcttgtcatttttttgcg

SEQ ID NO: 106 - DNA, 70 bp, synthetic construct
  source 1..70: mol_type = other DNA
cgaaagccatgacctccgatcactccgagctggtaaaagccgccctggacatgcgggtcgagcatcaggg

SEQ ID NO: 107 - DNA, 85 bp, synthetic construct
  source 1..85: mol_type = other DNA
atgtctctcacactcagtcatacctggaaatgccacatacgtacacggatctctaaggtaaagtgcttctcaataacatccgctc

SEQ ID NO: 108 - DNA, 75 bp, synthetic construct
  source 1..75: mol_type = other DNA
cgaaagccatgacctccgatcactcgcacagtgaagcatcggtcaatagcaagaaatcactgaagagcctctgac

SEQ ID NO: 109 - DNA, 80 bp, synthetic construct
  source 1..80: mol_type = other DNA
aggcccccattgcgaacggtctcatagtcatagtagaacgggtcccaatcagaaaccctacttgcctgctgcatgttgtg

SEQ ID NO: 110 - DNA, 70 bp, synthetic construct
  source 1..70: mol_type = other DNA
cgaaagccatgacctccgatcactcaggaggatgaggagccccacgatgaaggccagtccagcgaagatc

SEQ ID NO: 111 - DNA, 74 bp, synthetic construct
  source 1..74: mol_type = other DNA
tcccctcttcaaggggtctacatggcaactgtgaggaggctaggacgcaaatcacttgaagaagtgaaagcgag

SEQ ID NO: 112 - DNA, 69 bp, synthetic construct
  source 1..69: mol_type = other DNA
cgaaagccatgacctccgatcactctactttattgatggtacatgacaaggtgcggctccctaggcccc

SEQ ID NO: 113 - DNA, 70 bp, synthetic construct
  source 1..70: mol_type = other DNA
cgactgcccgcccatattgcacttggtcactacatctatttgtggcagaatatgtcgatgcaaaaaacct

SEQ ID NO: 114 - DNA, 72 bp, synthetic construct
  source 1..72: mol_type = other DNA
cgaaagccatgacctccgatcactcgacactagtacaaaataagttacttctggccgtggtgctccctgcag

SEQ ID NO: 115 - DNA, 85 bp, synthetic construct
  source 1..85: mol_type = other DNA
tttcctttgcagaggccaacagctttgaacaaatgattcgcctacaacttctttcgggttatatctatcatttacttgacaccct

SEQ ID NO: 116 - DNA, 75 bp, synthetic construct
  source 1..75: mol_type = other DNA
cgaaagccatgacctccgatcactcaccctcaacggcaactggtgaacggtaacactgattgcccagaactggta

SEQ ID NO: 117 - DNA, 85 bp, synthetic construct
  source 1..85: mol_type = other DNA
aagtttttcaatattttgtcgtacgttgtctcagctgcatcgcaagagcccgctttattatgtgttcgtctaactctgtttctgt

SEQ ID NO: 118 - DNA, 61 bp, synthetic construct
  source 1..61: mol_type = other DNA
cgaaagccatgacctccgatcactccctactttgtcactcaccagccttctatttctggat

SEQ ID NO: 119 - DNA, 85 bp, synthetic construct
  source 1..85: mol_type = other DNA
gcacacggtggcaaaacatgagtgggaatttcattctcagaccctgctggcttccttcctgtgttccagctacaaacttagaaac

SEQ ID NO: 120 - DNA, 73 bp, synthetic construct
  source 1..73: mol_type = other DNA
cgaaagccatgacctccgatcactctgtgtattgcccgacacattggatttcagcttctgtgcctgtaaattt

SEQ ID NO: 121 - DNA, 85 bp, synthetic construct
  source 1..85: mol_type = other DNA
ttgcctgggcaccactcttttgcttaaaagtagtgaagtcacgccaagtgctagcatctctgacgaaaacagcagacggaaaagt

SEQ ID NO: 122 - DNA, 64 bp, synthetic construct
  source 1..64: mol_type = other DNA
cgaaagccatgacctccgatcactcttgtcaaggagtttgcctaaggcgctccagtcacccatg

SEQ ID NO: 123 - DNA, 85 bp, synthetic construct
  source 1..85: mol_type = other DNA
atctgagcctgttatgaaaggtgtagctctgtcagcagattaagttcctgcatgaacgtgtcgtgttatgcagcggtatgtcgtg

SEQ ID NO: 124 - DNA, 75 bp, synthetic construct
  source 1..75: mol_type = other DNA
cgaaagccatgacctccgatcactcctactgagaaggaagcctgggtgaatacaaaggtcacagggagctgagtg

SEQ ID NO: 125 - DNA, 85 bp, synthetic construct
  source 1..85: mol_type = other DNA
ctaggtgcttggattgccgagttgagtttgctggaagggaaatggggcggcctaaattgggaaaaaaggttttagctattgatgg

SEQ ID NO: 126 - DNA, 72 bp, synthetic construct
  source 1..72: mol_type = other DNA
cgaaagccatgacctccgatcactccaacggaggctcagtccagacagggattaaccgacttgtgctggtat

SEQ ID NO: 127 - DNA, 85 bp, synthetic construct
  source 1..85: mol_type = other DNA
tgattgttattccaaaggccatctgaagactctgtcacggtgtcattccccctacatatataggaaaagggaaggtagaagagct

SEQ ID NO: 128 - DNA, 75 bp, synthetic construct
  source 1..75: mol_type = other DNA
cgaaagccatgacctccgatcactcctttagtggtattggccgtcagtagactatgttctaggaacagttgagtt

SEQ ID NO: 129 - DNA, 85 bp, synthetic construct
  source 1..85: mol_type = other DNA
gcagtattcacttttttggtttttatcttcagtgctggatctattttgatcctgttgcagtatcacgtaaatacctacttcgata

SEQ ID NO: 130 - DNA, 75 bp, synthetic construct
  source 1..75: mol_type = other DNA
cgaaagccatgacctccgatcactcaagtgaatggaagtcctttattcctagtacatctattagcacattggtct

SEQ ID NO: 131 - DNA, 85 bp, synthetic construct
  source 1..85: mol_type = other DNA
cgactccagaactccccgcccttggtgactgaggaaggaagggttggcccctcagcatctttagcagtagcacttgctaaattgg

SEQ ID NO: 132 - DNA, 64 bp, synthetic construct
  source 1..64: mol_type = other DNA
cgaaagccatgacctccgatcactccgcgggagtgggagacctggtggtaggctcttcgccttc

SEQ ID NO: 133 - DNA, 71 bp, synthetic construct
  source 1..71: mol_type = other DNA
tgcatagaagggaaactgggcttcgggctgcgcctcctggtcaagacttgcatgaggacccgcaaattcct

SEQ ID NO: 134 - DNA, 69 bp, synthetic construct
  source 1..69: mol_type = other DNA
cgaaagccatgacctccgatcactcagacaggagtccttgacatcgtgggagaggcattgtggcaatac

SEQ ID NO: 135 - DNA, 80 bp, synthetic construct
  source 1..80: mol_type = other DNA
gaagctattcagccactgtagttaagagctcctgttctgatccagcgattaagccgtagttgaatttatggagcggtgcc

SEQ ID NO: 136 - DNA, 70 bp, synthetic construct
  source 1..70: mol_type = other DNA
cgaaagccatgacctccgatcactcaagcccctcaacttgatcacggtgagaacacagggagcctttgga

SEQ ID NO: 137 - DNA, 85 bp, synthetic construct
  source 1..85: mol_type = other DNA
ccagtgaagccactgctttacacagaagatacacatagcttcctattgttcatttggtgttgcctagtactaggtgactggtacc

SEQ ID NO: 138 - DNA, 75 bp, synthetic construct
  source 1..75: mol_type = other DNA
cgaaagccatgacctccgatcactcacaccatctgtcatcatgactcaaagggaaatgctagccacaccattttt

SEQ ID NO: 139 - DNA, 85 bp, synthetic construct
  source 1..85: mol_type = other DNA
tcttttcccacaaggatttgcagccctcttccacctcaaagctacctccaccatgctgaaatcatggttgaagaagaagaagatc

SEQ ID NO: 140 - DNA, 75 bp, synthetic construct
  source 1..75: mol_type = other DNA
cgaaagccatgacctccgatcactcggtggctaggaggagtggaaagacactgatgccacctggaatcatagatt

SEQ ID NO: 141 - DNA, 85 bp, synthetic construct
  source 1..85: mol_type = other DNA
atcaaaacctgccctctcagacacatgcagacccaacagcagattttaaacatcaacaccaaacgcaatatcaggctgaatggtg

SEQ ID NO: 142 - DNA, 75 bp, synthetic construct
  source 1..75: mol_type = other DNA
cgaaagccatgacctccgatcactccgtcaaggtcaaggtttaaggcctttcgtagtcctgtcatttcaacaaat

SEQ ID NO: 143 - DNA, 85 bp, synthetic construct
  source 1..85: mol_type = other DNA
agggttagtccagacccacagtccacaaagacagcattaaagagggcttacctagttgacttggagtatgccatgaagactcgtc

SEQ ID NO: 144 - DNA, 60 bp, synthetic construct
  source 1..60: mol_type = other DNA
cgaaagccatgacctccgatcactctgggaaggcggaggctcaggagggcaggcaaccac

SEQ ID NO: 145 - DNA, 71 bp, synthetic construct
  source 1..71: mol_type = other DNA
tcagcttccatcgctgtgcagaggaaccaaccggggcgagctattgtaaatacgcagcaacaagcgtccag

SEQ ID NO: 146 - DNA, 60 bp, synthetic construct
  source 1..60: mol_type = other DNA
cgaaagccatgacctccgatcactccttcgtcaggcatattggtgaggctgacgggctgg

SEQ ID NO: 147 - DNA, 85 bp, synthetic construct
  source 1..85: mol_type = other DNA
caaacttctctgctccaactcagctccatcgccattggtctgacgcagcgcctcgaacagtgatacgcacactgataactatgcg

SEQ ID NO: 148 - DNA, 72 bp, synthetic construct
  source 1..72: mol_type = other DNA
cgaaagccatgacctccgatcactccggcagactcggcgcggctcgggcacctctcttccttatctcttact

SEQ ID NO: 149 - DNA, 75 bp, synthetic construct
  source 1..75: mol_type = other DNA
gctcctcaatgaagttccgcagctgtagccaaatggctccctgtttcattaatgtaacggacaggtcgctgaggt

SEQ ID NO: 150 - DNA, 75 bp, synthetic construct
  source 1..75: mol_type = other DNA
cgaaagccatgacctccgatcactccaggaagaacatgaactgcaccagcgtgctcgtccatgtgcctttcattt

SEQ ID NO: 151 - DNA, 82 bp, synthetic construct
  source 1..82: mol_type = other DNA
catccctctgtcaggcagagcctattataccccgattaccagaacagctctaacctgcatacatatggcatttagttgttca

SEQ ID NO: 152 - DNA, 75 bp, synthetic construct
  source 1..75: mol_type = other DNA
cgaaagccatgacctccgatcactctgagaactccagatgagttttgtggttgaaaagcctttcagttcttcctc

SEQ ID NO: 153 - DNA, 85 bp, synthetic construct
  source 1..85: mol_type = other DNA
cctttgtcttggatactatgggagcaaaacggtagccccaaagcacctcccgatacccgtagcgtgatggtcttatagctgctct

SEQ ID NO: 154 - DNA, 75 bp, synthetic construct
  source 1..75: mol_type = other DNA
cgaaagccatgacctccgatcactccacttccactgtcttgctaaagttatggaaatccactcggtatttccctt

SEQ ID NO: 155 - DNA, 85 bp, synthetic construct
  source 1..85: mol_type = other DNA
cactcacttccataatcgtcagtacatgccccgctttaattgtgtgattgcatacgaaatttgagcaagcaattgaaggcttaga

SEQ ID NO: 156 - DNA, 75 bp, synthetic construct
  source 1..75: mol_type = other DNA
cgaaagccatgacctccgatcactcctttgaaatgggattggtaaggatgacagtgtaatttcctgtgtctcttt

SEQ ID NO: 157 - DNA, 83 bp, synthetic construct
  source 1..83: mol_type = other DNA
ctgatggcattagattcctgcacgctaagagttctccctctacatctgctgtaacggtagatgggaaaaagtgtgctcatttt

SEQ ID NO: 158 - DNA, 75 bp, synthetic construct
  source 1..75: mol_type = other DNA
cgaaagccatgacctccgatcactcgtctttctcttcacctactgatggtttaggcgtgtgcatggctttgcctg

SEQ ID NO: 159 - DNA, 85 bp, synthetic construct
  source 1..85: mol_type = other DNA
tttggattcctcatttttcctgattatccactcacaagatgactcagttgcctacgtatatatccaagtggttatgtccgacggc

SEQ ID NO: 160 - DNA, 75 bp, synthetic construct
  source 1..75: mol_type = other DNA
cgaaagccatgacctccgatcactcaatgatagaagatccagtcacagaccccatctgttctttgatttttgtct

SEQ ID NO: 161 - DNA, 85 bp, synthetic construct
  source 1..85: mol_type = other DNA
aattgccagtttagaagcaattgtagcagcataggatggaggtggggttacatgttggagttaacggagacccgccatcgtttac

SEQ ID NO: 162 - DNA, 75 bp, synthetic construct
  source 1..75: mol_type = other DNA
cgaaagccatgacctccgatcactctggatgttttgtgagttaactggcagggtggtgggtaaatttggattgtg

SEQ ID NO: 163 - DNA, 73 bp, synthetic construct
  source 1..73: mol_type = other DNA
ggccaaccgggcatggacacggacagcaggtgttgttgcggttgttaatatgacaggccgctaaagacgttct

SEQ ID NO: 164 - DNA, 64 bp, synthetic construct
  source 1..64: mol_type = other DNA
cgaaagccatgacctccgatcactctggagcgagtggtgaagctcatgcccccagaaacggggt

SEQ ID NO: 165 - DNA, 85 bp, synthetic construct
  source 1..85: mol_type = other DNA
cttgactggcgagacatggtggcttgttcctggtggagcaagagaaccagccttcttgaagacctatgtaaagaaacgggtcact

SEQ ID NO: 166 - DNA, 60 bp, synthetic construct
  source 1..60: mol_type = other DNA
cgaaagccatgacctccgatcactctgaagctacgactgcccccgctccggaaggacaca

SEQ ID NO: 167 - DNA, 75 bp, synthetic construct
  source 1..75: mol_type = other DNA
cttcccatcacgtgtctcgatcttcttcacaaccacggcccgaacctaactcctcgctacattcctattgttttc

SEQ ID NO: 168 - DNA, 65 bp, synthetic construct
  source 1..65: mol_type = other DNA
cgaaagccatgacctccgatcactcctgttcacttgggcaggacgtcagaggactcagacaccag

SEQ ID NO: 169 - DNA, 85 bp, synthetic construct
  source 1..85: mol_type = other DNA
aataaatcttcagcagccttgagttcaagggtggcattttggtgcaactgcaatctatattccttacacgcttgcttgctcctac

SEQ ID NO: 170 - DNA, 75 bp, synthetic construct
  source 1..75: mol_type = other DNA
cgaaagccatgacctccgatcactcatacctccaattcttccagcggcttctggtaattttcctgaatttgtgac

SEQ ID NO: 171 - DNA, 85 bp, synthetic construct
  source 1..85: mol_type = other DNA
ctgtgctgatgctcaagtcattaccctctaagataatcataagctggagactttgacagagagtcaaaatcaataaactccgatc

SEQ ID NO: 172 - DNA, 75 bp, synthetic construct
  source 1..75: mol_type = other DNA
cgaaagccatgacctccgatcactccaatacattagtatgttcttcagatgggtgcaggtacacctcatcttggg

SEQ ID NO: 173 - DNA, 75 bp, synthetic construct
  source 1..75: mol_type = other DNA
ctcaaagccatttctccgctgacatccagtagtgctcttccaattaaagaacatgcgtaaaacgacctcgttgta

SEQ ID NO: 174 - DNA, 63 bp, synthetic construct
  source 1..63: mol_type = other DNA
cgaaagccatgacctccgatcactcagaggagccacagaggcagaaccgagcgccaggctgag

SEQ ID NO: 175 - DNA, 78 bp, synthetic construct
  source 1..78: mol_type = other DNA
gagggaagcagcagcgtttcggcagaacagggatgagctgctgccggtgcctgactaatcgtgtaggtgtctaataca

SEQ ID NO: 176 - DNA, 68 bp, synthetic construct
  source 1..68: mol_type = other DNA
cgaaagccatgacctccgatcactccgtggcagccacatggacgggctccgttgttatagaagaggga

SEQ ID NO: 177 - DNA, 82 bp, synthetic construct
  source 1..82: mol_type = other DNA
gcatgagcactggcctgactccgcaaagcaactgttgtttaaggctccgccgcgtttcgttgcgataacctcatatagtccc

SEQ ID NO: 178 - DNA, 75 bp, synthetic construct
  source 1..75: mol_type = other DNA
cgaaagccatgacctccgatcactctagtaaccaggttccacttcgttgcactgacgtccaatcatgtgaggccg

SEQ ID NO: 179 - DNA, 85 bp, synthetic construct
  source 1..85: mol_type = other DNA
aaatatcaagtacctggcatgtggttgcagagtcccttggatgcgatcatcgagcttggttctgtccctatagctacagctgtct

SEQ ID NO: 180 - DNA, 75 bp, synthetic construct
  source 1..75: mol_type = other DNA
cgaaagccatgacctccgatcactcttcagatgcagcttgtaggagataccaggctcaaggcagacaggattagg

SEQ ID NO: 181 - DNA, 85 bp, synthetic construct
  source 1..85: mol_type = other DNA
aacttcacccaaactagcttggcagtcaatcttcagtcctcttctgcaacccgtaggagtcggttgccactcgttgatagtcaac

SEQ ID NO: 182 - DNA, 75 bp, synthetic construct
  source 1..75: mol_type = other DNA
cgaaagccatgacctccgatcactcaaggaaaccaaaccaaaccaccccattgtggcctgagacttgctggagtg

SEQ ID NO: 183 - DNA, 85 bp, synthetic construct
  source 1..85: mol_type = other DNA
tgtgccggtaaaagccattcttgcacttctcgcagtgaatgccatcagtgcttactcttgccagtgggcatgaaattgcgagctt

SEQ ID NO: 184 - DNA, 75 bp, synthetic construct
  source 1..75: mol_type = other DNA
cgaaagccatgacctccgatcactcaagagaacctttggagttacaattgcagggcaaacagcggtccctttctc

SEQ ID NO: 185 - DNA, 85 bp, synthetic construct
  source 1..85: mol_type = other DNA
cgtaagctgctgactggatggattagacttgaacacgtgtgggtcatacactaatgatcccgcggtgtagtaattctacggaatg

SEQ ID NO: 186 - DNA, 72 bp, synthetic construct
  source 1..72: mol_type = other DNA
cgaaagccatgacctccgatcactccacacaggtggctctcacaacagctgtggactgtaatgtgtgtggac

SEQ ID NO: 187 - DNA, 85 bp, synthetic construct
  source 1..85: mol_type = other DNA
acacactcggccgtgcaagccaaaactcgcaccaggaaatcaaagtagggctgaggctgttaaagctgtagcaactcttccacga

SEQ ID NO: 188 - DNA, 75 bp, synthetic construct
  source 1..75: mol_type = other DNA
cgaaagccatgacctccgatcactcactcctgaagacaatcggcacgagctccccagtctctccaaaaagaggac

SEQ ID NO: 189 - DNA, 80 bp, synthetic construct
  source 1..80: mol_type = other DNA
agcaccaaacctagagccctcccctcgcacagtgtaatacacaaccagctcggattggatgtctcgtaaggaatacttat

SEQ ID NO: 190 - DNA, 75 bp, synthetic construct
  source 1..75: mol_type = other DNA
cgaaagccatgacctccgatcactcacaagattattgcggccggattcaaagttggggatgtaggcacgtttgat

SEQ ID NO: 191 - DNA, 85 bp, synthetic construct
  source 1..85: mol_type = other DNA
ggtttcttgtatggcagttacactggttgccatatccactgggtcaccaacttaaagctatccacgaatgtcaaaaatgtggttt

SEQ ID NO: 192 - DNA, 75 bp, synthetic construct
  source 1..75: mol_type = other DNA
cgaaagccatgacctccgatcactccctgtgtaaaatctcttagctgagaatgagtggtgtgggtttgctcctag

SEQ ID NO: 193 - DNA, 85 bp, synthetic construct
  source 1..85: mol_type = other DNA
aaaatgtcacaggagactttgaagaggcagacaggagggtggtggcagcactatcaattcgtgaccccgatcatccagtccagaa

SEQ ID NO: 194 - DNA, 75 bp, synthetic construct
  source 1..75: mol_type = other DNA
cgaaagccatgacctccgatcactcgcacgtatcttttaaggaatgacaccagttcctggcttctgaccaaagaa

SEQ ID NO: 195 - DNA, 85 bp, synthetic construct
  source 1..85: mol_type = other DNA
cctatgaagaattagggatgctccttggtccagagtagatgccaagaatgcattcgcaaccatgtgaagtaatgtgagcgtactt

SEQ ID NO: 196 - DNA, 75 bp, synthetic construct
  source 1..75: mol_type = other DNA
cgaaagccatgacctccgatcactcgcatgtcccccaaattgacttcagttggtatttcctgctttttaaaggtc

SEQ ID NO: 197 - DNA, 76 bp, synthetic construct
  source 1..76: mol_type = other DNA
agagaccagcttaactcagagcacactccctccaagcggtgcaccagttagcgtggcgtataccatgttgttaaca

SEQ ID NO: 198 - DNA, 75 bp, synthetic construct
  source 1..75: mol_type = other DNA
cgaaagccatgacctccgatcactcctgcgcagatccagcaaccttgtcaatactcatttttttccaggaccaga

SEQ ID NO: 199 - DNA, 85 bp, synthetic construct
  source 1..85: mol_type = other DNA
agagtccaagagaatggccgagttcatgagccgcaacacgatgtaagttgctatgataaatggttttcatcagccggattttgtt

SEQ ID NO: 200 - DNA, 75 bp, synthetic construct
  source 1..75: mol_type = other DNA
cgaaagccatgacctccgatcactcactgaaggtgtagctagggtacatcaaagccccgatatcagtagaatggg

SEQ ID NO: 201 - DNA, 75 bp, synthetic construct
  source 1..75: mol_type = other DNA
aatgctgattagctgtagagctgaaggcacggctgccaggcagcaagaaggagtatggaacttatagcaagagag

SEQ ID NO: 202 - DNA, 75 bp, synthetic construct
  source 1..75: mol_type = other DNA
cgaaagccatgacctccgatcactccaccgggaggagccactctctggaatcttaaattaccaggtaggagtgag

SEQ ID NO: 203 - DNA, 85 bp, synthetic construct
  source 1..85: mol_type = other DNA
gcactccacatctgggcttctgcattatttctatgacgcgggagtttaacctatcagctaatagggtcggctcaacagtgtatcc

SEQ ID NO: 204 - DNA, 75 bp, synthetic construct
  source 1..75: mol_type = other DNA
cgaaagccatgacctccgatcactcggaagtccattttgggctatttggaaatagtgagtattctgcaacatctg

SEQ ID NO: 205 - DNA, 85 bp, synthetic construct
  source 1..85: mol_type = other DNA
ggagatgcagacactgatgcagtcgtgggatgagtggacaatgaggagtgctcaagagcttgcgaacaagacggaagaagatatg

SEQ ID NO: 206 - DNA, 75 bp, synthetic construct
  source 1..75: mol_type = other DNA
cgaaagccatgacctccgatcactcggagagtggccctaatggtagtagaggcagctggagaagaaggaaaaaga

SEQ ID NO: 207 - DNA, 79 bp, synthetic construct
  source 1..79: mol_type = other DNA
cgcccgctgctatgggcaaagtttcgtggatgcggcaagggttgcgctatgcagacgagctggcagaggagagaaatca

SEQ ID NO: 208 - DNA, 65 bp, synthetic construct
  source 1..65: mol_type = other DNA
cgaaagccatgacctccgatcactccgtccttgctcgggtgttgtaagttccagtgcaaagtgcc

SEQ ID NO: 209 - DNA, 85 bp, synthetic construct
  source 1..85: mol_type = other DNA
aatgtcctctcgtctctggcttggcgaattgcccgtgatttttctagcagcgttgggaatgccatctctatcccttctttttcaa

SEQ ID NO: 210 - DNA, 75 bp, synthetic construct
  source 1..75: mol_type = other DNA
cgaaagccatgacctccgatcactccacttctcatcttctccttggctccagcaatcatgtagtaaaagatgtgg

SEQ ID NO: 211 - DNA, 85 bp, synthetic construct
  source 1..85: mol_type = other DNA
tcagtttcactcatcttcacacgtcttcaggttgcatgttcatggagtagcacccctccaaacgcattcttattggcaaatggaa

SEQ ID NO: 212 - DNA, 75 bp, synthetic construct
  source 1..75: mol_type = other DNA
cgaaagccatgacctccgatcactcggaggggagaggaaggattcagccagtgtccagactgaaattgagtaata

SEQ ID NO: 213 - DNA, 85 bp, synthetic construct
  source 1..85: mol_type = other DNA
ctgatccagtagtcacacttctttttcctaaacaggacggaggtgacattcacgcagctcttagcgcaatgtatgtgctgttatc

SEQ ID NO: 214 - DNA, 75 bp, synthetic construct
  source 1..75: mol_type = other DNA
cgaaagccatgacctccgatcactctaatgttgcccagcgtgaactcgccgggctggcaacctggaacaaaagtc

SEQ ID NO: 215 - DNA, 85 bp, synthetic construct
  source 1..85: mol_type = other DNA
tattaaggcccgaggcacccacgtcgagtgtgttaaagtgctgccgaggtcgctgggttaaatagagaaatggctactcatttag

SEQ ID NO: 216 - DNA, 75 bp, synthetic construct
  source 1..75: mol_type = other DNA
cgaaagccatgacctccgatcactcgtgcgaggatttgtgggaacttcttgaggacgccgatggcgaatgactgg

SEQ ID NO: 217 - DNA, 85 bp, synthetic construct
  source 1..85: mol_type = other DNA
aaatagacagaggcgtacatgtctgctgagaacctgtatgctcattcctgccttacgacttcactgcaattgacgattcagttaa

SEQ ID NO: 218 - DNA, 75 bp, synthetic construct
  source 1..75: mol_type = other DNA
cgaaagccatgacctccgatcactctgccatgtggctctgactgtatgaatgttgctcagaaccttattaccatc

SEQ ID NO: 219 - DNA, 85 bp, synthetic construct
  source 1..85: mol_type = other DNA
ctgtaatccactggttagtcttttaatttgccttttcagttcatcattctcctcataccaatgtaaagtatagttaacgccctgt

SEQ ID NO: 220 - DNA, 75 bp, synthetic construct
  source 1..75: mol_type = other DNA
cgaaagccatgacctccgatcactcctttggagttcttcaattagactttgtttattatctgtcaggggtttgcc

SEQ ID NO: 221 - DNA, 85 bp, synthetic construct
  source 1..85: mol_type = other DNA
aagtttagtttctaaagccaaccccaggatgcaccttgtttctactctggcgtgaaccagattatgtatggacgcgcaatagata

SEQ ID NO: 222 - DNA, 75 bp, synthetic construct
  source 1..75: mol_type = other DNA
cgaaagccatgacctccgatcactcagcatttcatttttgagacgacgtttacaatctgccctgtccttttggag

SEQ ID NO: 223 - DNA, 85 bp, synthetic construct
  source 1..85: mol_type = other DNA
cagaaccctggattgcaaagagcgcacttcaatggccttttggccccatcccccaacaaccgccggatatacgttgggatataaa

SEQ ID NO: 224 - DNA, 75 bp, synthetic construct
  source 1..75: mol_type = other DNA
cgaaagccatgacctccgatcactcgtgcacaggaatctcagcttcttaattgacctgcgcttcagctcactttc

SEQ ID NO: 225 - DNA, 85 bp, synthetic construct
  source 1..85: mol_type = other DNA
tgcccccaccctttgtgttcccaattccttccttagtgaatgaagaacttctgacacattagtaacgtcggcaagcacttagtcg

SEQ ID NO: 226 - DNA, 72 bp, synthetic construct
  source 1..72: mol_type = other DNA
cgaaagccatgacctccgatcactccatcattgaacttcaccttccctccaaccagttgccccaaactcccc

SEQ ID NO: 227 - DNA, 72 bp, synthetic construct
  source 1..72: mol_type = other DNA
ccacgcaggccctgacatcacatagtagatcgtcggccccctcttaactatagctccatttggacagacgtt

SEQ ID NO: 228 - DNA, 71 bp, synthetic construct
  source 1..71: mol_type = other DNA
cgaaagccatgacctccgatcactcctacttcgggtgggaagtcggggttctggaattgctgcatgagttg

SEQ ID NO: 229 - DNA, 85 bp, synthetic construct
  source 1..85: mol_type = other DNA
ctcactctccgcagtcagagttacaagcttatatggtcaaagacaaaaatcgcgatactgatgatgaaattttcaaaaatggctg

SEQ ID NO: 230 - DNA, 64 bp, synthetic construct
  source 1..64: mol_type = other DNA
cgaaagccatgacctccgatcactcctgccagtgctaaggacttcgttgcctctcctctctcca

SEQ ID NO: 231 - DNA, 85 bp, synthetic construct
  source 1..85: mol_type = other DNA
aaataaatgtagactttttgtggttctgttcagttcctgccggaggaggacaacagccactttttttccaaattttgcaagagcc

SEQ ID NO: 232 - DNA, 75 bp, synthetic construct
  source 1..75: mol_type = other DNA
cgaaagccatgacctccgatcactcctggtgggcttttgtgttgttttgttcctttttgcaaagaccatcatatt

SEQ ID NO: 233 - DNA, 85 bp, synthetic construct
  source 1..85: mol_type = other DNA
agagggaagttctgccattgaaaaaccagccgcatctcagatcccttcagcactacgatcccatgagaacacctgtagatactca

SEQ ID NO: 234 - DNA, 67 bp, synthetic construct
  source 1..67: mol_type = other DNA
cgaaagccatgacctccgatcactctcaggaagactctcagatgtcatggattcggagtcgcgctgc

SEQ ID NO: 235 - DNA, 71 bp, synthetic construct
  source 1..71: mol_type = other DNA
ctgcttgaagatggcatcacggccttccagctcattcctggtctaggtatctaattcgtgggtcgggtact

SEQ ID NO: 236 - DNA, 75 bp, synthetic construct
  source 1..75: mol_type = other DNA
cgaaagccatgacctccgatcactcgccactcttgtcatgaacccaaaggccctgttgaagtctttgaaatgaaa

SEQ ID NO: 237 - DNA, 85 bp, synthetic construct
  source 1..85: mol_type = other DNA
gagttaacagttcaaaagtatagctcttcacagcatcttcattttgagctcgtggtatccctggttgagacattggactagtgta

SEQ ID NO: 238 - DNA, 75 bp, synthetic construct
  source 1..75: mol_type = other DNA
cgaaagccatgacctccgatcactcagttgagccatggtgatcatcgaccaagtccagaatggatgaaggaactt

SEQ ID NO: 239 - DNA, 75 bp, synthetic construct
  source 1..75: mol_type = other DNA
ctggtgcaggctcctgaaggctcaggagaacagagggatgcctgaatcaatagaacaatatcagttatggcggtg

SEQ ID NO: 240 - DNA, 66 bp, synthetic construct
  source 1..66: mol_type = other DNA
cgaaagccatgacctccgatcactcgtcccagagtgggtaacagctgagtagaaggacaggcagga

SEQ ID NO: 241 - DNA, 80 bp, synthetic construct
  source 1..80: mol_type = other DNA
tggactgtgttgcttttcttgaccactttgtcaatacctgcagtgcttgagctctaggcccaaaacgaccttaatggtca

SEQ ID NO: 242 - DNA, 75 bp, synthetic construct
  source 1..75: mol_type = other DNA
cgaaagccatgacctccgatcactcgggcatcataagaaatcctgggctgggagagcatttcacatacgactatc

SEQ ID NO: 243 - DNA, 85 bp, synthetic construct
  source 1..85: mol_type = other DNA
gatggaaaatgatgcagccagcatctccgagaacaagctcacaggtggaacttggaggagttgatagtggtaaaacaacattagc

SEQ ID NO: 244 - DNA, 75 bp, synthetic construct
  source 1..75: mol_type = other DNA
cgaaagccatgacctccgatcactcgtggcatatacttttcctactgacactgcaatagcataagccaccacagc

SEQ ID NO: 245 - DNA, 83 bp, synthetic construct
  source 1..83: mol_type = other DNA
gcggccggcgaagctgaccacgatgaacatgccagcacacacatagatccttctggaatttcttcctttgattttgccatttt

SEQ ID NO: 246 - DNA, 75 bp, synthetic construct
  source 1..75: mol_type = other DNA
cgaaagccatgacctccgatcactcgtgaggcagagcaggaagaggaacatgtagacaatcttgtagaccacgag

SEQ ID NO: 247 - DNA, 75 bp, synthetic construct
  source 1..75: mol_type = other DNA
cggaaggtgtaagagatggtaggacccccagggatgggcgcatctccatgactgcttgagcggctggagaatctg

SEQ ID NO: 248 - DNA, 68 bp, synthetic construct
  source 1..68: mol_type = other DNA
cgaaagccatgacctccgatcactcctcgttctcagccgtgacgatgatattgaaggtgcccacggag

SEQ ID NO: 249 - DNA, 85 bp, synthetic construct
  source 1..85: mol_type = other DNA
attcaacagcactgcatgctcttttgcttattagcgctcaaaatgtcaccctttcgccacccatataaaccccacttcgtcctca

SEQ ID NO: 250 - DNA, 75 bp, synthetic construct
  source 1..75: mol_type = other DNA
cgaaagccatgacctccgatcactcaaaatatagcattaaacgacaggattctcttggttctccaagtgagggtt

SEQ ID NO: 251 - DNA, 85 bp, synthetic construct
  source 1..85: mol_type = other DNA
ataatatcaggctaacaccttccaaactagagcctcggatggtggcccagcaaggcagagcaaatgtgacactgtctatcagtac

SEQ ID NO: 252 - DNA, 75 bp, synthetic construct
  source 1..75: mol_type = other DNA
cgaaagccatgacctccgatcactctctgcttgaattgcttgtagcgacattgatggcacacgagtaagatccaa

SEQ ID NO: 253 - DNA, 85 bp, synthetic construct
  source 1..85: mol_type = other DNA
gagcgacccaggtagacgatgtagtcctccttctttgggtaatcaatgaacaaagcaaacctaatgaagccagacacgagatcac

SEQ ID NO: 254 - DNA, 75 bp, synthetic construct
  source 1..75: mol_type = other DNA
cgaaagccatgacctccgatcactcggttttccacctcaaacttcatctccccttgcgtgttggagttaagcctt

SEQ ID NO: 255 - DNA, 85 bp, synthetic construct
  source 1..85: mol_type = other DNA
ctcaatcagatgaaacctcaccatgcccactgcttaggagccttctacatctagcccagatcctacgagatgagctacgtaacta

SEQ ID NO: 256 - DNA, 75 bp, synthetic construct
  source 1..75: mol_type = other DNA
cgaaagccatgacctccgatcactcacgatggagaccatggcgaaagttcaacattccacacaggattccccctt

SEQ ID NO: 257 - DNA, 70 bp, synthetic construct
  source 1..70: mol_type = other DNA
ttggatcccgggcggcgtcatgcgcttctccttttccatacgcatgactacattacaacgggccaggaag

SEQ ID NO: 258 - DNA, 60 bp, synthetic construct
  source 1..60: mol_type = other DNA
cgaaagccatgacctccgatcactccccacctgcgagtagacgtcgtcgggcgtctgctg

SEQ ID NO: 259 - DNA, 75 bp, synthetic construct
  source 1..75: mol_type = other DNA
tggtggcagaatttcctctggctgctgtgacttcaagctgcctattgaagcaatcctctccccaatacttaaaaa

SEQ ID NO: 260 - DNA, 60 bp, synthetic construct
  source 1..60: mol_type = other DNA
cgaaagccatgacctccgatcactcggttcccagctggaggggctgcagacctgggaaaa

SEQ ID NO: 261 - DNA, 78 bp, synthetic construct
  source 1..78: mol_type = other DNA
ggagggaaagtcttggatgcacctggcactcaatccactcggcccagctgtctcaattattgctcaaaaaccactatt

SEQ ID NO: 262 - DNA, 65 bp, synthetic construct
  source 1..65: mol_type = other DNA
cgaaagccatgacctccgatcactccgcatgatccatcctgtcttcagtcagtgccttctggaag

SEQ ID NO: 263 - DNA, 85 bp, synthetic construct
  source 1..85: mol_type = other DNA
ctggacgttgatgccactgaatgcctggcagttttccacacagactttcccatcctcttcttttcttggtgttgagaagatgctc

SEQ ID NO: 264 - DNA, 75 bp, synthetic construct
  source 1..75: mol_type = other DNA
cgaaagccatgacctccgatcactcgtgaccacccctagcgtgctgcagttggcaccagaggaatgcagcttgta

SEQ ID NO: 265 - DNA, 85 bp, synthetic construct
  source 1..85: mol_type = other DNA
acatacttcactttgaagccttggtttattaatcaccaacacctgccagtctatcgtggacccaaagtcgatacgtccgattaag

SEQ ID NO: 266 - DNA, 75 bp, synthetic construct
  source 1..75: mol_type = other DNA
cgaaagccatgacctccgatcactccaggaaggtatgatgctatgcccacgttattccaaaagtcatctgcacac

SEQ ID NO: 267 - DNA, 85 bp, synthetic construct
  source 1..85: mol_type = other DNA
aagttccttgacaccgaggattaccaattgcttgccaatggagagtctgaccgggaatcggcatttcgcattcttaggatctaaa

SEQ ID NO: 268 - DNA, 75 bp, synthetic construct
  source 1..75: mol_type = other DNA
cgaaagccatgacctccgatcactcactgtgaacagctggacaagaacactggtctactcgccgaacttttttcc

SEQ ID NO: 269 - DNA, 75 bp, synthetic construct
  source 1..75: mol_type = other DNA
cctgaaagtcccaaggcatcagctgggacaggtttgaggacccgaagcaatactgtcgtcactctgtatgtccgt

SEQ ID NO: 270 - DNA, 65 bp, synthetic construct
  source 1..65: mol_type = other DNA
cgaaagccatgacctccgatcactcggctagggcagtattgtgagagggacctggagtatcattg

SEQ ID NO: 271 - DNA, 71 bp, synthetic construct
  source 1..71: mol_type = other DNA
ggcggaggagtaggggaggttctggacacggaacggctcctaaggttgctgatttggttgttggagaccca

SEQ ID NO: 272 - DNA, 75 bp, synthetic construct
  source 1..75: mol_type = other DNA
cgaaagccatgacctccgatcactcaaggcgaaaaccggagtcggaacttggcggtgggcggccaaggaagagaa

SEQ ID NO: 273 - DNA, 80 bp, synthetic construct
  source 1..80: mol_type = other DNA
agggaaaaggcctcaggagatggcctaggttcaaagtccctgtatcaaggcctagcctaaaggttcttgcagagcaacat

SEQ ID NO: 274 - DNA, 75 bp, synthetic construct
  source 1..75: mol_type = other DNA
cgaaagccatgacctccgatcactcttactagacgaaaagctgacacctgcagctcccactgacccacatgggcg

SEQ ID NO: 275 - DNA, 85 bp, synthetic construct
  source 1..85: mol_type = other DNA
ccatatacaacaaatccgatatggatccctttcttgccacgggaaggctcctcaggttgttacttgaagggttcaacacgagctc

SEQ ID NO: 276 - DNA, 75 bp, synthetic construct
  source 1..75: mol_type = other DNA
cgaaagccatgacctccgatcactctctaactagtaagacaggtgggaggttctttgtgaggatttccaaccaag

SEQ ID NO: 277 - DNA, 85 bp, synthetic construct
  source 1..85: mol_type = other DNA
gtggacagggatgtcaaagcctggagttactggcagtaaaaggtgagcaccattagctcggatgctatcagcttgcgcctattat

SEQ ID NO: 278 - DNA, 75 bp, synthetic construct
  source 1..75: mol_type = other DNA
cgaaagccatgacctccgatcactcctgtggactagaggcaatcaagttccaacttatcccacacactcagagct

SEQ ID NO: 279 - DNA, 85 bp, synthetic construct
  source 1..85: mol_type = other DNA
gacagtggagggcctctggtttgctttgagaaggacaaatacattttacacttaggctaccaaatgaatttaaagccagctgaaa

SEQ ID NO: 280 - DNA, 75 bp, synthetic construct
  source 1..75: mol_type = other DNA
cgaaagccatgacctccgatcactcggctggcagcctaaccctcacatgaatttttccctacctctatttagggt

SEQ ID NO: 281 - DNA, 85 bp, synthetic construct
  source 1..85: mol_type = other DNA
agcaccctgctaagcttttgggacccatcagaactatatttccataattgcctaattagctctaggaaacacaaccccgggattt

SEQ ID NO: 282 - DNA, 75 bp, synthetic construct
  source 1..75: mol_type = other DNA
cgaaagccatgacctccgatcactcaggattgatcagtgctccagttggtggagaaaacagcctagagatacgtt

SEQ ID NO: 283 - DNA, 72 bp, synthetic construct
  source 1..72: mol_type = other DNA
tcaggagcatcacggccaccatggatgttgaaggaggcttttttgcaattaaactgcccaggcgatctgttg

SEQ ID NO: 284 - DNA, 70 bp, synthetic construct
  source 1..70: mol_type = other DNA
cgaaagccatgacctccgatcactctggatgatgagcttcgtgaagggctctgtgatgatcttgagcagg

SEQ ID NO: 285 - DNA, 85 bp, synthetic construct
  source 1..85: mol_type = other DNA
acagagattatgggcacagattcaggacttgtctccgggacaatattagcccaatgcttgcagtatgtatcctgatcgtgcgtgc

SEQ ID NO: 286 - DNA, 75 bp, synthetic construct
  source 1..75: mol_type = other DNA
cgaaagccatgacctccgatcactcaggaccaaagaccgattctagcagcaatgacgcctgcaaacagcagactg

SEQ ID NO: 287 - DNA, 70 bp, synthetic construct
  source 1..70: mol_type = other DNA
accttcttggcacagcagcagtgtggcccacggagcacgatctgtattttgcacctttcgctatgctgag

SEQ ID NO: 288 - DNA, 65 bp, synthetic construct
  source 1..65: mol_type = other DNA
cgaaagccatgacctccgatcactcatgaagggttgtatgaggccccatcctggggaggctgtac

SEQ ID NO: 289 - DNA, 85 bp, synthetic construct
  source 1..85: mol_type = other DNA
tgtaggtcagctcctgactgacctctaccacgaagaagaagaggaagatgcttgagttatacggaacttcgcaaaagtattccct

SEQ ID NO: 290 - DNA, 75 bp, synthetic construct
  source 1..75: mol_type = other DNA
cgaaagccatgacctccgatcactcggagatcttctgggatttgggaaattcctcgtagccagggtcccagatgc

SEQ ID NO: 291 - DNA, 85 bp, synthetic construct
  source 1..85: mol_type = other DNA
aaacgatgatgtgctttttgaatggggttattcttactgtttgcatagggcgttcaatcgcttagtttcgtggcgggatttgagg

SEQ ID NO: 292 - DNA, 75 bp, synthetic construct
  source 1..75: mol_type = other DNA
cgaaagccatgacctccgatcactcatcccaagcaacggcaacatgggactgagacacatgttaatgcaaatgga

SEQ ID NO: 293 - DNA, 85 bp, synthetic construct
  source 1..85: mol_type = other DNA
cggtgactgcgtcgttcagcagcgactccccgaagacgatgatgaacaggccatccatcaacaactgctccaacagcctttccat

SEQ ID NO: 294 - DNA, 75 bp, synthetic construct
  source 1..75: mol_type = other DNA
cgaaagccatgacctccgatcactcgttgtcacctcccagcgccacgaaagattcaaacacattgtacagaacca

SEQ ID NO: 295 - DNA, 80 bp, synthetic construct
  source 1..80: mol_type = other DNA
gattggggtcggagggcttcctgacgaggaaagagcgcggcatagcattacgactttacgagttcgcagaacaaagactt

SEQ ID NO: 296 - DNA, 70 bp, synthetic construct
  source 1..70: mol_type = other DNA
cgaaagccatgacctccgatcactctaaactctggattagagtcctgcagctcgctgtagttaggcttcc

SEQ ID NO: 297 - DNA, 85 bp, synthetic construct
  source 1..85: mol_type = other DNA
atcctgccgccgccgatgattgttattattattttttggaaaggcttaagccgatcttcataacggacaaactgaacgggccatt

SEQ ID NO: 298 - DNA, 75 bp, synthetic construct
  source 1..75: mol_type = other DNA
cgaaagccatgacctccgatcactcggcaaactggaatcaggatcaaaaaaagcgcttccctcctcctctggccg

SEQ ID NO: 299 - DNA, 85 bp, synthetic construct
  source 1..85: mol_type = other DNA
tttctcgttgatttcgctgctccatttagccaaggttggcctggccactgcacaattctgcgggttagcaggaaggttagggaac

SEQ ID NO: 300 - DNA, 75 bp, synthetic construct
  source 1..75: mol_type = other DNA
cgaaagccatgacctccgatcactcattctccatcatcctccacgcttgctctgaagagggtttaaaaagtccag

SEQ ID NO: 301 - DNA, 85 bp, synthetic construct
  source 1..85: mol_type = other DNA
ctgactcgtttcataactgtccttcccacggctgtcccaatcagaaggcgcactggttggttatccaatgatcgacctggagtct

SEQ ID NO: 302 - DNA, 60 bp, synthetic construct
  source 1..60: mol_type = other DNA
cgaaagccatgacctccgatcactcttgtggctgtgggtttcagcactctggtcatccag

SEQ ID NO: 303 - DNA, 85 bp, synthetic construct
  source 1..85: mol_type = other DNA
ggatctggatacactgaaagcttaggtgaggatttgatgaggatttttttcttttaacaaacactggctttttccatcttgttgc

SEQ ID NO: 304 - DNA, 60 bp, synthetic construct
  source 1..60: mol_type = other DNA
cgaaagccatgacctccgatcactcttcctctttccctctcctggcttgagtgaagatgt

SEQ ID NO: 305 - DNA, 75 bp, synthetic construct
  source 1..75: mol_type = other DNA
acaacagttgccggtgggttctgattgacatcggtggctgcaaatgcactctatatggagggagagtagctggat

SEQ ID NO: 306 - DNA, 69 bp, synthetic construct
  source 1..69: mol_type = other DNA
cgaaagccatgacctccgatcactcctgaggaattctgttggatgctagaaagtggcaggctctgtggg

SEQ ID NO: 307 - DNA, 85 bp, synthetic construct
  source 1..85: mol_type = other DNA
aagacatccaaactgcgttcaggcagctgaaacaggcttctttcccagtgccgtctcagatgagtgggttaatcaatcaagtatg

SEQ ID NO: 308 - DNA, 75 bp, synthetic construct
  source 1..75: mol_type = other DNA
cgaaagccatgacctccgatcactcggcaagctgaagaataaatagactgagtttccgggcaatgtctgtcctca

SEQ ID NO: 309 - DNA, 85 bp, synthetic construct
  source 1..85: mol_type = other DNA
ctactgaaattcctcaccgtgttatttaacaccatacaggcctaggaactctatgtcgtgctataatggcgtctgtcgtgctcat

SEQ ID NO: 310 - DNA, 75 bp, synthetic construct
  source 1..75: mol_type = other DNA
cgaaagccatgacctccgatcactcgctttggcctttgcactatagctcccaaaagacagattgctggagataac

SEQ ID NO: 311 - DNA, 85 bp, synthetic construct
  source 1..85: mol_type = other DNA
cacaaaaggctgcacaggtgattacaggccaagtaggaaggtctgtggcaccatccactttcatggaaacaataagagcagggaa

SEQ ID NO: 312 - DNA, 75 bp, synthetic construct
  source 1..75: mol_type = other DNA
cgaaagccatgacctccgatcactcacactgaataaccccaagcagacggagttcttgacagagttttgaaggcc

SEQ ID NO: 313 - DNA, 85 bp, synthetic construct
  source 1..85: mol_type = other DNA
ccttctgctctcctgaagccaccttgctagatgggaactgagacatgattctggaatatcctcatgctcatagatgtcaaatttc

SEQ ID NO: 314 - DNA, 69 bp, synthetic construct
  source 1..69: mol_type = other DNA
cgaaagccatgacctccgatcactcgctggaagggaggctgggtcttttcttatcttcagactggtcct

SEQ ID NO: 315 - DNA, 85 bp, synthetic construct
  source 1..85: mol_type = other DNA
gaggcttaaaactgccagtgtcatttacatccttcttggtctctaaggatcatacgccttacgccccaaggtttagcgtatcaaa

SEQ ID NO: 316 - DNA, 75 bp, synthetic construct
  source 1..75: mol_type = other DNA
cgaaagccatgacctccgatcactcgggtttgggaccaatgatgggagcacctgaaggtttagatgctacttctg

SEQ ID NO: 317 - DNA, 81 bp, synthetic construct
  source 1..81: mol_type = other DNA
aagctatgttcctccttatgatttagttctgcagcatcactctgatctagctcaattgccattgctggaatagtgtcggta

SEQ ID NO: 318 - DNA, 75 bp, synthetic construct
  source 1..75: mol_type = other DNA
cgaaagccatgacctccgatcactcctgcgcttttcaagtccttcttactgctagaagatggatcttgaacatgc

SEQ ID NO: 319 - DNA, 85 bp, synthetic construct
  source 1..85: mol_type = other DNA
actgctgacacctcccttgaagctgagatgggaaatggacatacttagaacgctcattttgaacatacgattgcgattacggaaa

SEQ ID NO: 320 - DNA, 75 bp, synthetic construct
  source 1..75: mol_type = other DNA
cgaaagccatgacctccgatcactcacagactcctccagagtagagcttggagggagattgaaagtggagataat

SEQ ID NO: 321 - DNA, 70 bp, synthetic construct
  source 1..70: mol_type = other DNA
tcctggaaacggtgccggtgctgcagagcccgcgacagcgaatctgcaactaacgcaagttacatcctag

SEQ ID NO: 322 - DNA, 68 bp, synthetic construct
  source 1..68: mol_type = other DNA
cgaaagccatgacctccgatcactcaaaaagaaagcgcccaacggctggacgcacaccccgccaggcc

SEQ ID NO: 323 - DNA, 85 bp, synthetic construct
  source 1..85: mol_type = other DNA
tcatagtttccagcaaaccggaacatctggacggaaaatcggccctgggacgtcgtgtcttagacgactgtgtgtgattctcgag

SEQ ID NO: 324 - DNA, 75 bp, synthetic construct
  source 1..75: mol_type = other DNA
cgaaagccatgacctccgatcactctttcattcatggtgtcacagagatagacttcacagtgcaggtagactagg

SEQ ID NO: 325 - DNA, 85 bp, synthetic construct
  source 1..85: mol_type = other DNA
gctcaaatggcaatcacaaacggttcagcaagaatggcgatgcctgccatctcatgtcctctgttaatccagcctgaatatgcca

SEQ ID NO: 326 - DNA, 75 bp, synthetic construct
  source 1..75: mol_type = other DNA
cgaaagccatgacctccgatcactcgacagtaaagtgaccgtgacagaactctcatggcaatagaggcttccaga

SEQ ID NO: 327 - DNA, 85 bp, synthetic construct
  source 1..85: mol_type = other DNA
tgcttcaggaagaggttgggtgtgaaaaagtcttgttgtgttgctgctgtcaaactggagagagaagtgaagacgatttaaccca

SEQ ID NO: 328 - DNA, 75 bp, synthetic construct
  source 1..75: mol_type = other DNA
cgaaagccatgacctccgatcactcactggtcatcattgtttggagggctgttgttttggtacctctctagcatc

SEQ ID NO: 329 - DNA, 75 bp, synthetic construct
  source 1..75: mol_type = other DNA
acgaggtttgtcacctggtatgcactgagccgagtgactgcgattgctgcattccgctcaacgcttgaggaagta

SEQ ID NO: 330 - DNA, 69 bp, synthetic construct
  source 1..69: mol_type = other DNA
cgaaagccatgacctccgatcactctccccttcttcactaggtaggaaatgtagaatttggttcctggc

SEQ ID NO: 331 - DNA, 71 bp, synthetic construct
  source 1..71: mol_type = other DNA
gggcctctttgctgtcaaacatgcagagaggcttttcgccctggaacaacggttatattctctggcagaac

SEQ ID NO: 332 - DNA, 60 bp, synthetic construct
  source 1..60: mol_type = other DNA
cgaaagccatgacctccgatcactccaggacatacaggtagacctcgtgggtgccagtga

SEQ ID NO: 333 - DNA, 85 bp, synthetic construct
  source 1..85: mol_type = other DNA
attttccaatattctgggtagagtacagtcatgagttcatctacactggaccgagtgcatgagctgtctttcacatgatacatcg

SEQ ID NO: 334 - DNA, 75 bp, synthetic construct
  source 1..75: mol_type = other DNA
cgaaagccatgacctccgatcactccctgttctctgttatgttgccagcctcctttccttagctgacacttgtac

SEQ ID NO: 335 - DNA, 85 bp, synthetic construct
  source 1..85: mol_type = other DNA
cgaatgtgcggactttttgttagcaagaaacttctgcagcctttggagagctgtgacatcgcttctagagtatgagccgcaatgc

SEQ ID NO: 336 - DNA, 75 bp, synthetic construct
  source 1..75: mol_type = other DNA
cgaaagccatgacctccgatcactcgacaagagcgcccctaagtttttaataactcgctaaagcctgtctttgct

SEQ ID NO: 337 - DNA, 80 bp, synthetic construct
  source 1..80: mol_type = other DNA
caaagcacaagccgcctcccatgcctggcatctggaattcacaagcctggagtttatgtattgccaacgagtttgtcttt

SEQ ID NO: 338 - DNA, 60 bp, synthetic construct
  source 1..60: mol_type = other DNA
cgaaagccatgacctccgatcactccagaccttctgttcctggtggcttccaagtgaagg

SEQ ID NO: 339 - DNA, 85 bp, synthetic construct
  source 1..85: mol_type = other DNA
cagcctgggaactggtctggagaggccagccggcgtagcttttctgtgtcctgttgagattattgagcttcatcatgaccagaag

SEQ ID NO: 340 - DNA, 75 bp, synthetic construct
  source 1..75: mol_type = other DNA
cgaaagccatgacctccgatcactctaatttgtgtcaacatctgtccccaccgccccagggcccagcggcagacc

SEQ ID NO: 341 - DNA, 76 bp, synthetic construct
  source 1..76: mol_type = other DNA
cttggtctcccttatttccctccatgtctttctcactctgccaaagacgcctatcttccagtttgatcgggaaact

SEQ ID NO: 342 - DNA, 60 bp, synthetic construct
  source 1..60: mol_type = other DNA
cgaaagccatgacctccgatcactcctctctcccgtccaaaatttcagtcctgctgtgct

SEQ ID NO: 343 - DNA, 85 bp, synthetic construct
  source 1..85: mol_type = other DNA
tgacctcgggaatgttagacaagattcacccccgatgccttgctctctgacgctcacgtgatctaccctagctgaccgctaatga

SEQ ID NO: 344 - DNA, 75 bp, synthetic construct
  source 1..75: mol_type = other DNA
cgaaagccatgacctccgatcactcaaaagttgcctggcagaactacatcctgctttccaggttagcagcctggc

(19) United States
(12) Patent Application Publication
(10) Pub. No.: US 2025/0304893 A1
(43) Pub. Date: Oct. 2, 2025

(54) PERFUSABLE 3D TUBULE-ON-CHIP MODEL DERIVED FROM KIDNEY ORGANOIDS WITH IMPROVED DRUG UPTAKE

(71) Applicants: President and Fellows of Harvard College, Cambridge, MA (US); The General Hospital Corporation, Boston, MA (US)

(72) Inventors: Jeffrey O. ACEVES, Cambridge, MA (US); Kayla WOLF, Cambridge, MA (US); Sanlin S. ROBINSON, Cambridge, MA (US); Ryuji MORIZANE, Charlestown, MA (US); Jennifer A. LEWIS, Cambridge, MA (US); Ronald VAN GAAL, Cambridge, MA (US)

(73) Assignees: PRESIDENT AND FELLOWS OF HARVARD COLLEGE, CAMBRIDGE, MA (US); THE GENERAL HOSPITAL CORPORATION, BOSTON, MA (US)

(21) Appl. No.: 19/107,722

(22) PCT Filed: Sep. 1, 2023
(86) PCT No.: PCT/US2023/073271

§ 371 Date: Feb. 28, 2025

Related U.S. Application Data
(60) Provisional application No. 63/403,175, filed on Sep. 1, 2022.

(51) Int. Cl. C12M 3/00; C12N 5/071

(52) U.S. Cl. CPC C12M 21/08; C12N 5/0686; C12N 2506/45; C12N 2513/00; C12N 2533/54; C12N 2533/90

(57) ABSTRACT

Described herein are perfusable 3D tubule-on-chip models comprising at least one tubule consisting of one patent lumen circumscribed by organoid-derived cells, and a multifluidic platform comprising at least one individually addressable chip. The models may further include an unseeded tubule, where the seeded tubule and the unseeded tubule are co-localized on the chip, and wherein the tubule and the unseeded tubule are embedded within a gelatin-fibrin extracellular matrix (ECM). Also, described here are methods of producing the described perfusable 3D tubule-on-chip models, and uses of the same.

### RELATED APPLICATIONS

The present patent document claims the benefit of the filing date under 35 U.S.C. § 119(e) of Provisional U.S. Patent Application Ser. No. 63/403,175, filed Sep. 1, 2022, which is hereby incorporated by reference.

### FEDERALLY SPONSORED RESEARCH OR DEVELOPMENT

This invention was made with government support under contract number TR002155 awarded by National Institutes of Health (NIH). The government has certain rights in this invention.

### REFERENCE TO SEQUENCE LISTING SUBMITTED VIA PATENTCENTER

This application is being filed electronically via PatentCenter and includes an electronically submitted Sequence Listing in XML format. The .XML file contains a sequence listing “514968_5000257_SL.xml” created on Aug. 14, 2023 and is 323,350 bytes in size. The sequence listing contained in this .xml file is part of the specification and is hereby incorporated by reference in its entirety.

### BACKGROUND

Each human kidney is composed of roughly one million nephrons that filter blood and maintain electrolyte homeostasis by reabsorbing necessary nutrients back into the blood. These respective functions are achieved by glomerular and tubular subunits that reside within each nephron. The first segment of the nephron's tubular network is known as the convoluted proximal tubule (PT). The PT is responsible for about 60-80% of nutrient reabsorption into the surrounding peritubular capillary network (Eaton, et al. 2009), making it highly susceptible to damage from drugs and toxins. Chronic and acute kidney injury are on the rise due to increased use of prescription drugs. While roughly 25% of acute renal failure is drug induced (Eric, et al. 2011), predicting nephrotoxicity in preclinical in vitro human models or animal studies remains difficult. Currently, renal toxicity accounts for only 2% of failures in preclinical drug testing, yet it is responsible for nearly 20% of failures in Phase III clinical trials (Tiong H Y, et al. 2014; Eric D., et al. 2011; Choudhury D., et al. 2006). Hence, there is a critical need for patient-specific, in vitro models that more faithfully recapitulate the proximal tubule segment within human kidneys.

When cultured in two-dimensions (2D), proximal tubule epithelial cells (PTECs) typically exhibit loss of polarization and function due to limited transporter expression in the absence of physiologic cues induced by extracellular matrices and fluid flow. To overcome these limitations, considerable effort has been devoted to developing more predictive 3D models of nephrotoxicity (Yu P, et al. 2022; Wilmer M J, et al. 2016; Lee J., et al. 2018; and van Duinen V, et al. 2015). Tubular networks of PTECs grown within a 3D matrigel environment form highly differentiated tubules that respond more sensitively to known nephrotoxins compared to PTECs cultured in 2D (Secker P F, et al. 2018). However, these tubular networks cannot be readily perfused. To enable fluid flow, 3D microfluidic (Wilmer M J, et al. 2016; Jang K J, et al. 2013; and Vormann M K, et al. 2021) and bioprinted (Homan K A, et al. 2016; and Lin N Y C, et al. 2019) PT models have recently been introduced that exhibit enhanced polarization, function, and maturation compared to 2D culture methods. Human primary and immortalized PT cells have been used in these models; however, both cell types come with their own limitations. Primary cell lines have limited potential for self-renewal and vary considerably based on the donor (PromoCell. Human Primary Cells and Immortal Cell Lines: Differences and Advantages. Blog Lab (2019)), while immortalized PT cell lines lack proper transporter expression compared to their in-vivo counterparts (Jenkinson S E, et al. 2012). Hence, there is considerable interest in using renewable cell sources that are patient-specific to accurately predict nephrotoxicity in preclinical drug screening models.

Another promising approach for predicting nephrotoxicity and engineering kidney tissues is the differentiation of nephron-rich kidney organoids from human pluripotent stem cells (hPSCs) (Kim Y K, 2018; Homan K A, et al. 2019; Nieskens T T G, et al. 2016; Wieser M, et al. 2008; Morizane R, et al. 2017; Hale L J, et al. 2018; and Takasato et al., 2016). Kidney organoids, often referred to as “mini-organs in a dish” have been shown to elicit injury responses when exposed to known nephrotoxins (Kim et al.). Moreover, when exposed to superfusive flow, kidney organoids exhibit enhanced vascularization and maturation compared to those cultured under static conditions (Homan et al.).

Also, to date, no scalable method has been introduced for successfully perfusing fluid through organoid-derived tubular segments. Understanding how flow influences drug transport and uptake within individual PT segments in kidney organoids is a crucial step towards establishing their potential for drug screening, disease modeling, and, ultimately, engineering kidney tissue for therapeutic use.

Also, although a few groups have attempted to make different perfusable models of collecting ducts on chip from other species, to date, there are no human ureteric bud or collecting duct models on chip. While these models have been valuable for studies, human cells and potentially patient-specific cells are ideal for studying human diseases and for tissue engineering.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. Proximal tubule epithelial cells in kidney organoids. (a) Schematic overview of process used to create 3D organoid-derived proximal tubule epithelial cell (3D OPTECs)-on-chip models. (b) Confocal image of kidney organoid (tissue-cleared) stained for PODXL⁺ (red), LTL⁺ (green), and CDH1⁺ (magenta), scale bar=100 μm. (c) Schematic showing marker localization in different segments of the nephron. (d) Higher magnification, confocal image of kidney organoid (cryo-sectioned) stained for LTL⁺ and CDH1⁺ regions, scale bar=10 μm. (e-g) Confocal images of kidney organoid (cryo-sectioned) stained for AQP1⁺, LRP2⁺, and SLC3A1⁺ (magenta) in nephron segments, scale bars=50 μm. (h) Heat map comparing transporter expression of OPTECs isolated from kidney organoids at days 21, 35, 49, 84, and 105. (i, j) Confocal images of kidney organoid (day 49, cryosectioned) stained for OCT2⁺ (magenta), LTL⁺ (green), and CDH1⁺ (cyan), scale bar=100 μm. (k, l) Higher magnification, confocal images of kidney organoid (day 49, cryo-sectioned) stained for OCT2⁺ (magenta) and LTL+(green), scale bar=10 μm.

FIG. 2. Optimizing OPTEC expansion. (a) Brightfield images showing OPTECs grown on plastic, matrigel, and laminin-511 substrates. (b) Heat map showing transporter expression of OPTECs between passages 0 (after isolation) and 5. (c) Brightfield images showing OPTEC populations at passages 0-5, scale bar=20 μm. (d) Schematic view of the extracellular matrix (ECM), media, and media supplement conditions explored for optimizing OPTEC culture conditions. (e) Heat map comparing transporter expression as a function of the experimental conditions tested in (d).

FIG. 3. 3D OPTEC-on-chip model. (a) Schematic views showing the processing steps used to create multiplexed, 3D OPTEC-on-chip models. (b) Corresponding images (left to right) of a representative chip after placing the channel templates, infilling the chip with ECM, removing the templates to create two-colocalized channels, and seeding one channel with OPTECs to create a 3D proximal tubule. (c) Confocal image of 3D OPTEC tubule showing actin (red) and DAPI (blue), scale bar=50 μm. (d) Cross-sectional image of 3D OPTEC tubule (day 14, perfusion) highlighting the formation of a confluent monolayer, scale bar=50 μm. (e) Brightfield image of 3D OPTEC tubule upon reaching confluency (day 7), scale bar=75 μm. (f) OPTEC tubule (day 14, perfusion) stained for Na⁺/K⁺ ATPase (green), LTL (magenta), and DAPI (blue). (g) OPTEC tubules exhibit proper apical polarization of primary cilia marker, acetylated alpha tubulin (red). (h, i) Basement membrane proteins laminin (red) and Col IV (green) are deposited by OPTECs on chip. (j) Proper expression of AQP1 (yellow) is observed in OPTEC tubules. (f, j) scale bars=20 μm. (k) SEM image highlighting primary cilia on OPTEC, scale bar=5 μm. (l) SEM image of OPTEC brush border, scale bar=20 μm. (m) TEM image of brush border, scale bar=1 μm.

FIG. 4. Improved transporter expression and polarization of 3D OPTECs-on-chip. (a) Heat map showing OCT2, OAT1, and OAT3 transporter expression for OPTECs and PTEC-TERT1 s-on-chip (day 0, as seeded), after achieving confluency (day 7, perfusion), and one-week after achieving confluency on chip (day 14, perfusion). (b) General transporter analysis comparing OPTEC tubules normalized by PTEC-TERT1 tubules after day 14 of perfusion on chip, one sample t test, n=6 tubules across 3 batches of OPTECs, *p<0.05, **p<0.01, ***p<0.005. (c, d) Immunofluorescence images showing OCT2 (green) and DAPI (blue) staining in OPTEC tubules after day 14 of perfusion on chip. (e-f) Immunofluorescence images of showing localization of OAT3 (green) and DAPI (blue) in OPTEC tubules after day 14 of perfusion on chip (g, h), Immunofluorescence images showing OCT2 (green) and DAPI (blue) staining in PTEC-TERT1 tubules after day 14 of perfusion on chip, and (i, j) Immunofluorescence images of showing localization of OAT3 (green) and DAPI (blue) in PTEC-TERT1 tubules after day 14 of perfusion on chip, scale bars=20 μm.

FIG. 5. Nephrotoxicity testing. (a) Comparison of OCT2, OAT1, and OAT3 transporter expression in OPTEC tubules (n=6) after day 14 of perfusion on chip compared PTEC-TERT1 tubules (controls), one sample t test. (b) Schematic view of experimental step used to introduce drugs and corresponding inhibitors as well as collect luminal perfusate. (c) Schematic view of OCT2-mediated uptake of cisplatin and inhibition using cimetidine. (d) Normalized LDH release observed after dosing the OPTEC (n=4-5) and PTEC-TERT1 (n=3-4) tubules on chip with cisplatin for 48 h, two-way ANOVA. (e) Schematic view of OAT1/3-mediated uptake of aristolochic acid and inhibition using probenecid. (f) Normalized LDH release observed after dosing the OPTEC (n=4-10) and PTEC-TERT1 (n=3-9) tubules on chip with aristolochic acid for 48 h, two-way ANOVA, *p<0.05, **p<0.01, ***p<0.005, ****p<0.001.

FIG. 6. OPTEC culture. (a-i) 2D culture of OPTECs on laminin 111, laminin 511, and laminin 111/511-coated substrates at varying concentrations of 1 μg/mL, 2.5 μg/mL, and 5 μg/mL. (j) Plot of number of OPTECs at day 11 when cultured on 2.5 μg/mL of laminin 111, laminin 511, or laminin 111/511. (k) OPTECs undergo dedifferentiation (epithelial to mesenchymal transition) of during prolonged culture. (l) OPTECs treated with 10 μM of TGF-β inhibitor, SB431542, exhibit cuboidal cell morphology during prolonged cell culture, scale bars=100 μm.

FIG. 7. Optimized matrix rheology. Semilog plot of storage (G′) and loss (G″) moduli of our optimized extracellular matrix (ECM), which is composed of 1 wt % gelatin and 20 mg fibrinogen.

FIG. 8. Multiplexed 3D proximal tubule-on-chip device fabrication. (a) Polycarbonate housing. (b)O-rings are placed into machined indentions within each chip to create a proper seal. (c) Fishing line, which serves as a channel template, is threaded through two sets of pins on each chip in the device. (d) Glass slides (50 mm×75 mm) are placed into a steel base plate and the polycarbonate housing with O-rings and fishing line is screwed on using 12 mm M4 screws. (e) A gelatin-fibrinogen solution is cast into each chip to fully encapsulate the templated channels. (f) After enzymatically cross-linking this solution, the fishing line is removed leaving behind empty channels embedded within a gelatin-fibrin matrix that can be seeded with cells.

FIG. 9. Proximal tubules of varying diameter in multiplexed chip devices. (Top row) Bright field and confocal images of proximal tubules produced using (100 μm diameter fishing line) in our multiplexed chip device, which shows (from left to right) empty channel (Day −1), seeding of PTEC-TERTs (Day 0), formation of confluent epithelium (Day 4), and confocal image of proximal tubules (Day 6) stained for different functional markers (Na+/K+ ATPase, LTL, AQP1, and merged). (Middle row) Corresponding image sequence for proximal tubules produced using fishing line that is 200 μm in diameter. (Bottom row) Corresponding image sequence for proximal tubules produced using fishing line that is 300 μm in diameter. Scale bars=200 μm (bright-field images) and 20 μm (confocal images).

FIG. 10. Multicellular aggregates. (a) Immunofluorescence image of a OPTEC monolayer in 2D culture stained for Na/K ATPase (green), LTL (magenta), CDH1 (red), and DAPI (blue). (b) Confocal image of OPTEC tubule-on-chip without flushing step, which reveals the presence of multicellular aggregates. In (a-b), all aggregates observed express CDH1. (c) Confocal image of OPTEC tubule improved protocol with intermittent flushing steps that mitigate multicellular aggregate formation, all scale bars=100 μm.

FIG. 11. Diffusional permeability measurements. A 4.5 kDa FITC-inulin dye in LPTEC media is perfused through luminal channels at varying times: (a) t=0 min and (b) t=15 min for confluent OPTEC tubules, and (c) t=0 min and (d) t=15 min for control channels (unseeded). (e) Diffusional permeability for tubules seeded with PTEC-TERT1 s or OPTECs as well as the control (unseeded channel), scale bar=300 μm, n=3, two-way ANOVA, **p<0.01.

FIG. 12. Drug transporter expression. Heat map showing expression of OCT2, OAT1, and OAT3 assessed via Nanostring for OPTEC and PTEC-TERT1 tubules (n=4-6) perfused on chip from day 0 through day 28. All data normalized to PTEC-TERT1 transporter expression on day 0 (immediately after seeding).

FIG. 13. Validation of UB organoid culture. A) Differentiation scheme in 96-well compared to μ-well culture. B) UB and stemness markers in iPSCs compared to UB organoids at 21 days and 35 days in 96-well and μ-well culture. C) UB in 96-well vs. iPSC and UB in μ-well vs. iPSC culture show upregulation of UB markers and downregulation of stemness markers. D) Gene expression remains relatively stable over time in both 96-well and μ-well formats. E) 96-well vs. μ-well shows minimal differences in gene expression at 21 days. F) UB organoids (day 21) in μ-wells show expression of broad UB markers such as ETV5, KRT8, and E-CAD.

FIG. 14. UB organoids can be differentiated to CD organoids by A) removing UB growth factors and culturing with aldosterone and vasopressin for at least 5 days. B) CD organoids show downregulation of UB marker RET and upregulation of CD marker KRT8 relative to UB organoids. AQP2 is expressed in both UB and CD organoids.

FIG. 15. ECM optimization. A) Assay to test monolayer formation on ECM involves dissociating UB organoids (day 21-35) and seeding them as single cells onto −500 μm thick ECM gels with confluency quantification after 2 days. B) Morphology of cells grown on gelbrin, BsM, collagen I, and BsM+ collagen I. Blue =DAPI, Green=Phalloidin, scale=50 μm. C) Quantification of cell confluency after 2 days. *p<0.05,****p<0.0001 with one-way ANOVA followed by Tukey-Kramer test for multiple comparisons. D) Storage modulus (G′) of BsM compared to BsM-collagen I, ***p<0.001 by t-test. E) Morphology of cells grown on gels with methacrylated HA interpenetrating network. Blue=DAPI, Green=phalloidin 488, scale=50 μm.

FIG. 16. Chip fabrication and validation. A) Design and perfusion of custom chip with pin-pullout method for fabricating pre-cast channels. B) Cross-section view showing single perfusable channel cast within ECM in the inner reservoir which is in contact with medium above the ECM in the outer reservoir. C) Cells seeded in the channel form a confluent monolayer within 7 days. D) Immunostaining confluent UB channel that was perfused for 4 days. E) Single plane cross-section of channel demonstrating monolayer formation. F) Epithelialized channels retain FITC-inulin compared to empty channels. G) Diffusion profile of FITC-inulin is reduced in epithelialized channel relative to empty channel. H) UB cells perfused on-chip show epithelial morphology with lateral CDH1 (cross-section view), apical BK-alpha and primary cilia (cross-section view), and basolateral NaK⁺-ATPase (top view).

FIG. 17. A) Assembly of chip. B) Fabrication of open channel within chip.

### BRIEF SUMMARY

Described herein are perfusable 3D tubule-on-chip models.

Specifically, described herein is a perfusable 3D proximal tubule-on-chip model. The described model exhibits superior drug transport over proximal tubule models used in industry, containing immortalized cell lines. The described model may be useful for pharmaceutical companies as a drug screening tool.

Also, described herein are perfusable models of the ureteric bud (UB) and collecting duct (CD).

One embodiment relates to a perfusable 3D tubule-on-chip model comprising organoid-derived cells and a multifluidic platform comprising at least one individually addressable chip, wherein the chip comprises a first channel consisting of one patent lumen circumscribed by the organoid-derived cells, and wherein the first channel is embedded within an extracellular matrix (ECM). In the perfusable 3D tubule-on-chip model, the organoid-derived cells are organoid-derived proximal tubule epithelial cells (OPTECs) isolated from kidney organoids derived from human pluripotent stem cells (hPSCs); or the organoid-derived cells are ureteric bud (UB) cells isolated from UB organoids derived from hiPSCs. In the perfusable 3D tubule-on-chip model, the chip may further comprise a second channel, the second channel being empty (non-seeded), wherein the first channel and the second channel are co-localized on the chip; and wherein the second channel is embedded within the ECM. In the perfusable 3D tubule-on-chip model, the multifluidic platform can comprise at least two individually addressable chips. In the perfusable 3D tubule-on-chip model, the multifluidic platform cam comprise 6 to 10 individually addressable chips. In the perfusable 3D tubule-on-chip model, the ECM can comprise at least one of gelatin and fibrinogen. In the perfusable 3D tubule-on-chip model, the ECM comprises 20 mg/mL fibrinogen. In the perfusable 3D tubule-on-chip model, the second channel may be seeded with endothelial cells thereby creating a vascularized 3D tubule-on-chip model.

Another embodiment relates to a perfusable 3D proximal tubule-on-chip model comprising organoid-derived proximal tubule epithelial cells (OPTECs) and a multifluidic platform comprising at least one individually addressable chip, wherein the chip comprises a first channel consisting of one patent lumen circumscribed by the OPTECs, and wherein the first channel is embedded within an extracellular matrix (ECM). In the perfusable 3D proximal tubule-on-chip model, the chip may further comprise a second channel, the second channel being empty (non-seeded), wherein the first channel and the second channel are co-localized on the chip; and wherein the second channel is embedded within the ECM. In the perfusable 3D proximal tubule-on-chip model, the OPTECs may be isolated from kidney organoids derived from human pluripotent stem cells (hPSCs). In the perfusable 3D proximal tubule-on-chip model, the multifluidic platform can comprise at least two individually addressable chips. In the perfusable 3D proximal tubule-on-chip model the multifluidic platform can comprise 6 to 10 individually addressable chips. In the perfusable 3D proximal tubule-on-chip model, the ECM can comprise at least one of gelatin and fibrinogen. In the perfusable 3D proximal tubule-on-chip model the ECM comprises 20 mg/mL fibrinogen. In the perfusable 3D proximal tubule-on-chip model, the OPTECs exhibit: at least 1.5-fold higher drug transporter expression, as compared to an immortalized proximal tubule epithelial cell line; and/or at least 2-fold higher drug uptake, as compared to an immortalized proximal tubule epithelial cell line. In the perfusable 3D proximal tubule-on-chip model, the OPTECs exhibit a higher expression of basolateral drug transporters OCT2, OAT1, and OAT3, as compared to an immortalized proximal tubule epithelial cell line. In the perfusable 3D proximal tubule-on-chip model, the first channel can exhibit a higher cell death response to known nephrotoxins, cisplatin and aristolochic acid, compared to an immortalized proximal tubule epithelial cell line. In the perfusable 3D proximal tubule-on-chip model, the second channel may be seeded with endothelial cells thereby creating a vascularized OPTEC-on-chip model.

Another embodiment relates to a perfusable 3D ureteric bud-on-chip model comprising organoid-derived ureteric bud (UB) cells and a multifluidic platform comprising at least one individually addressable chip, wherein the chip comprises a first channel consisting of one patent lumen circumscribed by the organoid-derived UB cells, and wherein the first channel is embedded within an extracellular matrix (ECM). In the perfusable 3D ureteric bud-on-chip model, the chip can further comprise a second channel, the second channel being empty (non-seeded), wherein the first channel and the second channel are co-localized on the chip; and wherein the second channel is embedded within the ECM. In the perfusable 3D ureteric bud-on-chip model, the organoid-derived UB cells may be isolated from ureteric bud organoids derived from human pluripotent stem cells (hPSCs). In the perfusable 3D ureteric bud-on-chip model, the multifluidic platform can comprise at least two individually addressable chips. In the perfusable 3D ureteric bud-on-chip model, the multifluidic platform can comprise 6 to 10 individually addressable chips. In the perfusable 3D ureteric bud-on-chip model, the ECM can comprise at least one of methacrylated hyaluronic acid, collagen, Matrigel, and polylysine. In the perfusable 3D ureteric bud-on-chip model, the ECM comprises 1% w/v methacrylated hyaluronic acid, 1.5 mg/mL collagen I and, optionally, coated with at least one of Matrigel and polylysine. In the perfusable 3D ureteric bud-on-chip model, the organoid-derived UB cells exhibit: epithelial morphology with lateral CDH1 expression, apical BK-alpha expression, primary cilia, and NaK⁺-ATPase.

Another embodiment relates to a method of producing a perfusable 3D kidney-on-chip model comprising: (i) isolating organoid-derived cells from an organoid derived from human pluripotent stem cells (hPSCs); (ii) seeding the isolated organoid-derived cells onto a multifluidic platform comprising at least one individually addressable chip, wherein the chip contains a first channel consisting of one patent lumen, wherein the organoid-derived cells are seeded within the first channel and circumscribe the first channel. In the method, the organoid derived cells can be: organoid-derived proximal tubule epithelial cells (OPTECs) from a kidney organoid derived from human pluripotent stem cells (hPSCs); or ureteric bud (UB) cells isolated from UB organoids derived from hiPSCs. In the method, the chip can further comprise a second channel, the second channel being empty (non-seeded), wherein the first channel and the second channel are co-localized on the chip; and wherein the first and the second channels are embedded within the ECM. The method may further comprise seeding the second channel with endothelial cells thereby creating a vascularized 3D kidney-on-chip model. In the method, the step of isolating the organoid-derived cells may be by magnetic-activated cell sorting. In the method, the isolated OPTECs may be LTL+ OPTECs. The method may further comprise expanding the organoid-derived cells in 2D culture. The method may further comprise differentiating hPSCs into nephron progenitor cells; producing kidney organoids from the nephron progenitor cells; and maturing the kidney organoids under static culture conditions. In the method, the first channel may be coated with laminin-511. In the method, the chip may be produced by: encapsulating a first channel template within an ECM solution cast into the chip; enzymatically cross-linking the ECM solution; and removing the first channel template, thereby forming the first channel, where the first channel can be seeded with organoid-derived cells. The method may further comprise encapsulating a second channel template within an ECM solution cast into the chip; removing the second channel template, thereby forming the second channel. In the method, a minimum seeding density of organoid-derived cells may be 10 million cells/mL. In the method, the ECM solution may be a gelatin-fibrinogen solution.

Another embodiment relates to the use of the perfusable 3D tubule-on-chip model described herein in drug toxicity studies.

Another embodiment relates to the use of the perfusable 3D tubule-on-chip model described herein in polarized drug uptake studies.

Another embodiment relates to the use of the perfusable 3D tubule-on-chip model described herein in personalized drug screening.

Yet another embodiment relates to the use of the perfusable 3D tubule-on-chip model described herein in disease modeling.

Yet another embodiment relates to a perfusable 3D proximal tubule-on-chip model comprising: (i) an OPTEC tubule consisting of one patent lumen circumscribed by organoid-derived proximal tubule epithelial cells (OPTECs); (ii) a multifluidic platform comprising at least one individually addressable chip, wherein the OPTEC tubule is embedded within an extracellular matrix (ECM). The perfusable 3D proximal tubule-on-chip model may further comprise an unseeded tubule, wherein the OPTEC tubule and the unseeded tubule are co-localized on the chip; and wherein the unseeded tubule is embedded within the ECM. In the perfusable 3D proximal tubule-on-chip model, the OPTECs may be isolated from kidney organoids derived from human pluripotent stem cells (hPSCs). In the perfusable 3D proximal tubule-on-chip model, the multifluidic platform can comprise at least 2 individually addressable chips. In the perfusable 3D proximal tubule-on-chip model, the multifluidic platform can comprise 6 to 10 individually addressable chips. In the perfusable 3D proximal tubule-on-chip model, the ECM can comprise at least one of gelatin and fibrinogen. In the perfusable 3D proximal tubule-on-chip model, the ECM comprises 20 mg/mL fibrinogen. In the perfusable 3D proximal tubule-on-chip model, the OPTEC tubule exhibits: at least 1.5-fold higher drug transporter expression, as compared to a tubule with an immortalized proximal tubule epithelial cell line; and/or at least 2-fold higher drug uptake, as compared to a tubule with an immortalized proximal tubule epithelial cell line. In the perfusable 3D proximal tubule-on-chip model, the OPTECs exhibit a higher expression of basolateral drug transporters OCT2, OAT1, and OAT3, as compared to an immortalized proximal tubule epithelial cell line. In the perfusable 3D proximal tubule-on-chip model, the OPTEC tubule exhibits a higher cell death response to known nephrotoxins, cisplatin and aristolochic acid, compared to an immortalized proximal tubule epithelial cell line.

### DETAILED DESCRIPTION OF THE DRAWINGS AND THE PRESENTLY PREFERRED EMBODIMENTS

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, compositions, devices and materials are described herein.

All patents, patent applications and publications, and other literature references cited herein are hereby incorporated by reference in their entirety. The disclosures of these publications in their entireties are hereby incorporated by reference into this application in order to more fully describe the state of the art as known to those skilled therein as of the date of the invention described and claimed herein.

As used herein and in the appended claims, the singular forms “a,” “and,” and “the” include plural referents unless the context clearly dictates otherwise. Thus, for example, reference to “a protein” includes a plurality of such proteins and reference to “the progenitor cell” includes reference to one or more progenitor cells known to those skilled in the art, and so forth.

### INTRODUCTION

Described herein is an integrated multifluidic platform that combines organoid-derived tubule cells, such as proximal tubule epithelial cells (OPTECs) and/or ureteric bud (UB) cells with a perfusable 3D kidney-on-chip model to enable, e.g., personalized drug toxicity testing.

The organoid-derived cells can be isolated from kidney organoids derived from hPSCs and seeded within a multifluidic platform composed of at least one individually addressable chip, each chip containing one channel, or two or more co-localized channels.

Each model can include of one patent lumen circumscribed by organoid-derived cells and one empty (non-seeded) channel, embedded within a gelatin-fibrin extracellular matrix (ECM). The organoid-derived cells, such as OPTECs form a confluent monolayer within ˜7 days and exhibit proper apical and basal polarization, as demonstrated by acetylated alpha tubulin and LTL expression and Na⁺/K⁺ ATPase expression, basement membrane protein deposition, and basal expression of transporters in the organic cation and anion transporter families, respectively.

In certain embodiments, the incorporation of two or more independently addressable channels per chip allows nephrotoxic drugs, cisplatin and aristolochic acid, to be introduced basolaterally, mimicking the native uptake of nephrotoxic substances in human kidneys. The effect of these drugs can then be studied. For example, reported herein are the surprising and unexpected effects of luminal flow on kidney organoid-derived proximal tubule maturation and functional response.

Also, described herein are methods for producing perfusable 3D kidney-on-chip models that combine organoid-derived cells, such as proximal tubule epithelial cells (OPTECs) and/or ureteric bud (UB) cells with an integrated multifluidic platform.

Also, described herein are methods for developing a collecting duct (CD) that is scalable, perfusable and derived from hPSCs using scalable culture methods to achieve sufficient yield for the scalable fabrication of engineered kidney tissues, be supported by a scaffold that promotes CD differentiation, and consist of a branching network with a single drainage outlet.

Engineered, patient-specific tubules described herein, such as proximal tubules and collecting ducts on-chip could expedite drug screening, disease modeling, and kidney biomanufacturing.

Functional models of kidney tissue or parts of kidney tissue, such as the collecting duct network, are ideal for screening drug efficacy and toxicity to limit animal testing and late-stage drug failure.

### Perfusable 3D Tubule-On-Chip Model

Certain embodiments relate to a perfusable 3D tubule-on-chip model comprising (i) organoid-derived cells and (ii) a multifluidic platform comprising at least one individually addressable chip. The organoid-derived cells may be, e.g., OPTECs isolated from kidney organoids derived from human pluripotent stem cells (hPSCs) (one type of differentiation protocol); or the organoid-derived cells may be UB cells isolated from ureteric bud (UB) organoids derived from hiPSCs (second type of differentiation protocol).

Certain alternative embodiments relate to a perfusable 3D proximal tubule-on-chip model comprising (i) OPTECs and (ii) a multifluidic platform comprising at least one individually addressable chip, wherein the chip comprises a first channel consisting of one patent lumen circumscribed by the OPTECs, and wherein the first channel is embedded within an extracellular matrix (ECM).

Certain alternative embodiments relate to a perfusable 3D ureteric bud-on-chip model comprising: (i) organoid-derived UB cells; and (ii) a multifluidic platform comprising at least one individually addressable chip, wherein the chip comprises a first channel consisting of one patent lumen circumscribed by the organoid-derived UB cells, and wherein the first channel is embedded within an ECM.

### (I) Organoid-Derived Cells

The term “organoid” refers to an “embryoid body” whose cells have undergone a degree of differentiation. The term “embryoid body” refers to a plurality of cells containing pluripotent or multipotent stem cells formed into a three-dimensional sphere, spheroid, or other three-dimensional shape. It is acknowledged that the distinction between an organoid and embryoid body remains undefined, and the use of the terms should be considered interchangeable.

An organoid may be created by culturing at least one of: pluripotent stem cells, multipotent stem cells, progenitor cells, nephron progenitor cells, terminally differentiated cells, endothelial cells, endothelial progenitor cells, immortalized cell lines, or primary cells. In certain embodiments, the population of cells comprises at least one of human embryonic stem cells (hESCs) or induced pluripotent stem cells (hiPSCs).

In certain embodiments, the OPTECs and or UB cells are isolated from kidney organoids derived from hPSCs. The term “organoid-derived proximal tubule epithelial cells (OPTECs)” refers to epithelial cells isolated from kidney organoids; the term “organoid-derived UB cells” refers to epithelial cells isolated from ureteric bud organoids or kidney organoids. For example, in certain embodiments, the UB cells can be obtained from kidney organoids that are specifically UB, or kidney organoids that are not specifically UB.

The cells may be cultured for at least 1 day and can be cultured indefinitely, and until the culturing is no longer desired. In some embodiments, cultures of cells can be grown for 30 days or longer, e.g., the cells may be cultured for 2 months, 3 months, 6 months, 9 months, 12 months, 24 months, 30 months, 36 months, 42 months, etc. Any time periods in between the mentioned time periods for culturing the cells are also contemplated. For example, in certain embodiments, the cells may be cultured for at least 2 days, at least 3 days, at least 4 days, at least 5 days, at least 6 days, at least 7 days, at least 8 days, at least 9 days, at least 10 days, at least 11 days; at least 12 days; at least 13 days; at least 14 days; at least 15 days; at least 16 days; at least 17 days; at least 18 days; at least 19 days; at least 20 days; at least 21 days; at least 22 days; at least 23 days; at least 24 days; at least 25 days; at least 26 days; at least 27 days; at least 28 days; at least 29 days; at least 30 days; or at least 31 days; or longer.

In certain embodiments, the organoid may be a kidney organoid.

In certain other embodiment, the organoid may be ureteric bud organoid.

In certain embodiments, the OPTECs and/or organoid-derived UB cells are isolated from kidney organoids and seeded within the multifluidic platform.

In certain embodiments, the seeding density of OPTECs should be at least 1 M cells/ML; preferably in a rage of 1-50 M cells/mL.

In certain alternative embodiments, a minimum seeding density of the OPTECs and/or organoid-derived UB cells should be 1 million (M) cells/mL; 2 M cells/mL; 5 M cells/mL; 8 M cells/mL; 10 M cells/mL; or 15 M cells/mL. For UB, lower amounts can be problematic as cells require cell-cell contacts to thrive. In view of this, a minimum seeding density of the OPTECs and/or organoid-derived UB cells should be at least 1 million (M) cells/mL; at least 10 M cells/mL; or at least 15 M cells/mL

In certain embodiments, the OPTECs exhibit:
(i) insignificantly different drug transporter expression, as compared to an immortalized proximal tubule epithelial cell line; at least 1.5-fold higher drug transporter expression, as compared to an immortalized proximal tubule epithelial cell line; at least 2-fold higher drug transporter expression, as compared to an immortalized proximal tubule epithelial cell line; or at least 3-fold higher drug transporter expression, as compared to an immortalized proximal tubule epithelial cell line; and/or
(ii) insignificantly different drug uptake, as compared to an immortalized proximal tubule epithelial cell line; at least 2-fold higher drug uptake, as compared to an immortalized proximal tubule epithelial cell line; at least 3-fold higher drug uptake, as compared to an immortalized proximal tubule epithelial cell line; at least 4-fold higher drug uptake, as compared to an immortalized proximal tubule epithelial cell line; or at least 5-fold higher drug uptake, as compared to an immortalized proximal tubule epithelial cell line.

In certain embodiments, the OPTECs of the perfusable 3D proximal tubule-on-chip model described herein exhibit a higher expression of basolateral drug transporters OCT2, OAT1, and OAT3, as compared to an immortalized proximal tubule epithelial cell line. The term “higher” with reference to the expression of the basolateral drug transporters, e.g., OCT2, OAT1, and OAT3 refers to an increased protein expression by at least 1.2-fold; at least 1.2-fold; or at least 1.4-fold, as compared to an immortalized proximal tubule epithelial cell line (“higher” refers to the RNA data (Nanostring) showing that the OPTECs on chip expressed higher levels of drug transporters than the age matched PTEC TERT chips).

In certain embodiment, organoid-derived UB cells of the perfusable 3D UB-on-chip model exhibit apical expression of potassium transporter BK alpha and primary cilia and lateral expression of CDH1.

### (II) Multifluidic Platform

As noted above, the perfusable 3D proximal tubule-on-chip model also comprises a multifluidic platform comprising at least one individually addressable chip.

The term “multifluidic platform” refers to a platform that allows to perform a set of fluidic unit operations that are enabled by a set of fluidic elements, which are designed for easy combination with a well-defined fabrication technology. A microfluidic platform includes at least one individually addressable, microfluidic chip. In some embodiments, as described herein, multifluidic platform refers to the fact that there are multiple chips included within one device for the setup. For example, each multifluidic platform used can contain 6 “gels”, where each gel is comprised of the two tubules (e.g., 1 OPTEC tubule and 1 empty tubule for drug delivery) used for the study. In certain embodiments, the multifluidic platform can include, e.g., 6 gels equaling 6 separate OPTEC tubules and their corresponding empty channel tubules.

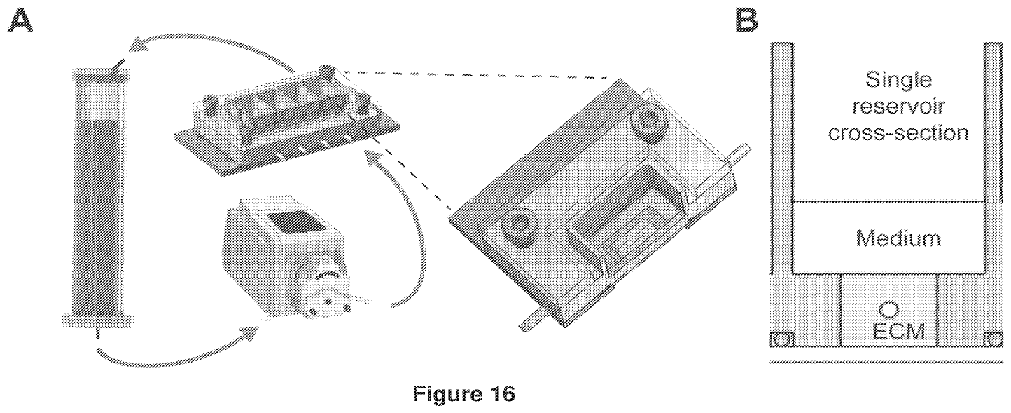

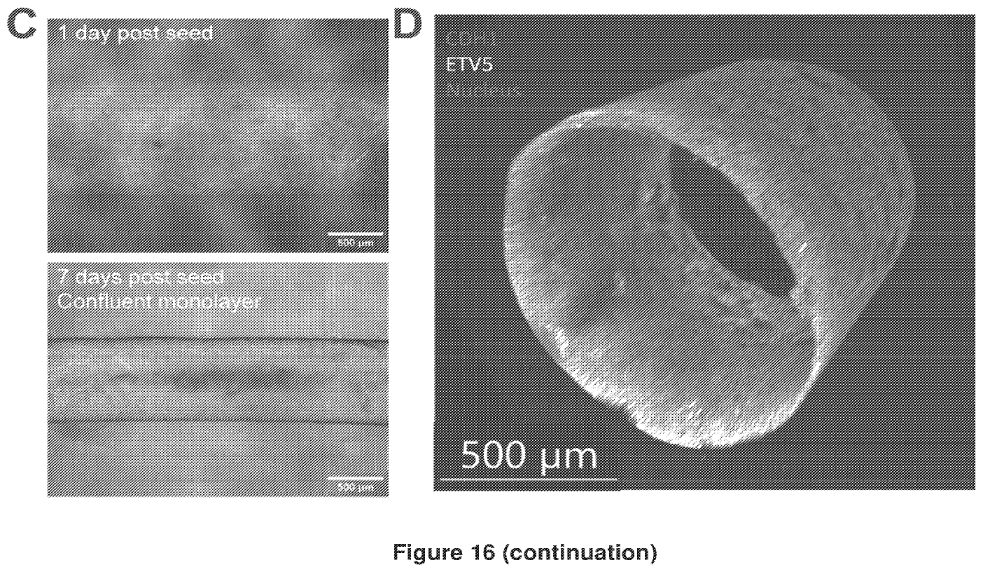

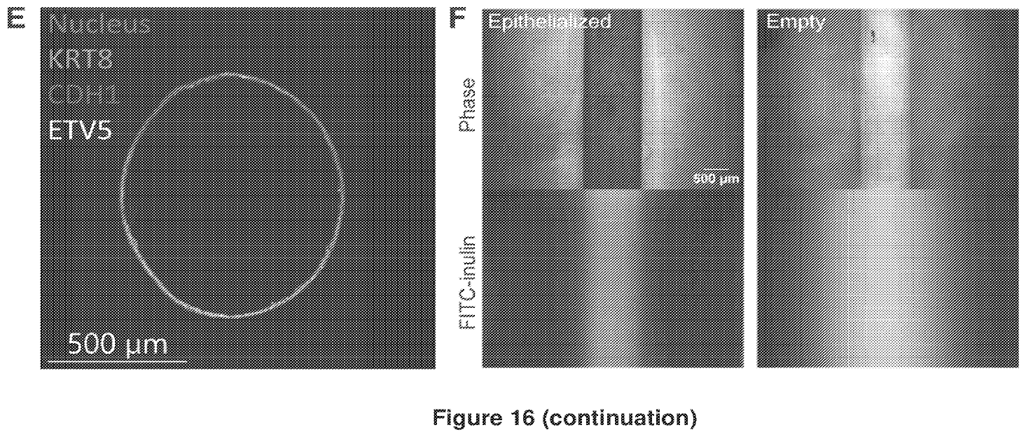

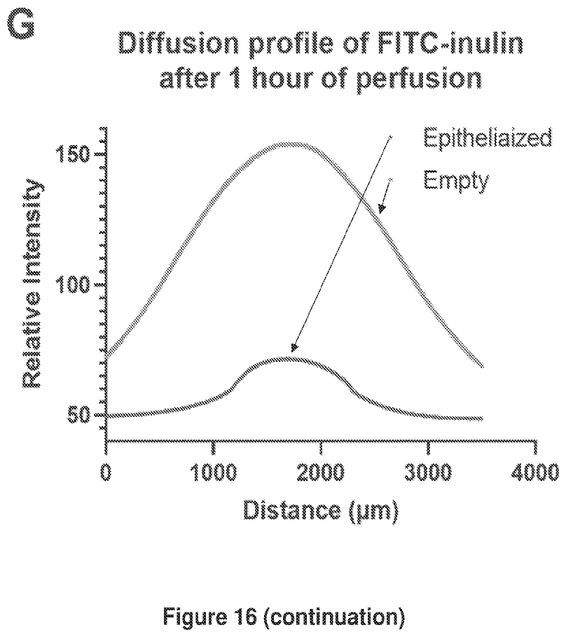

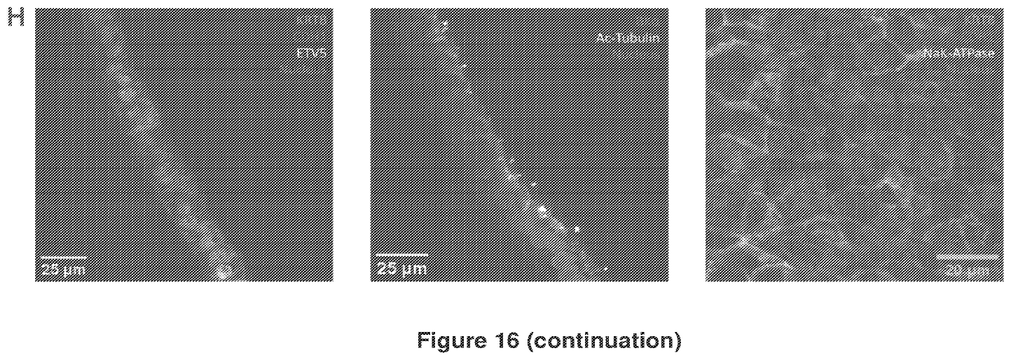

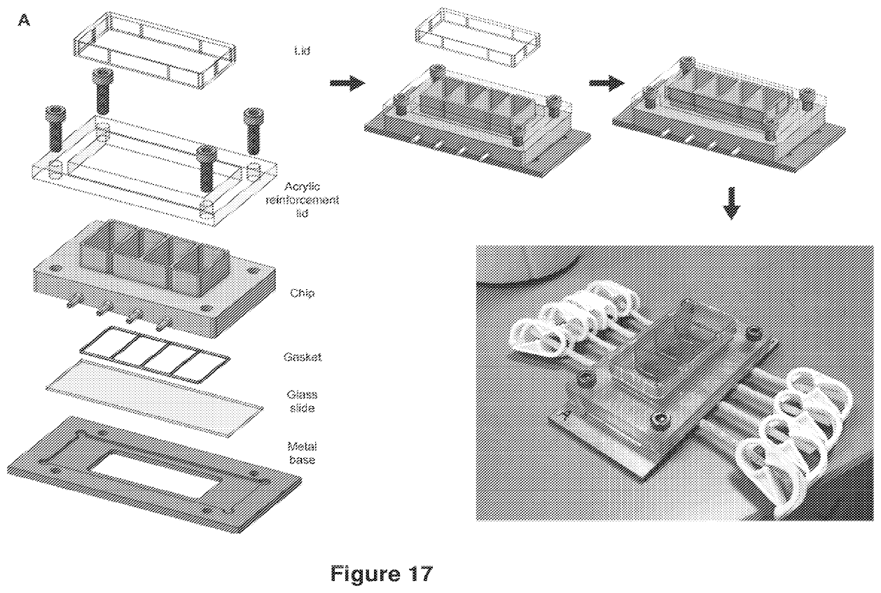

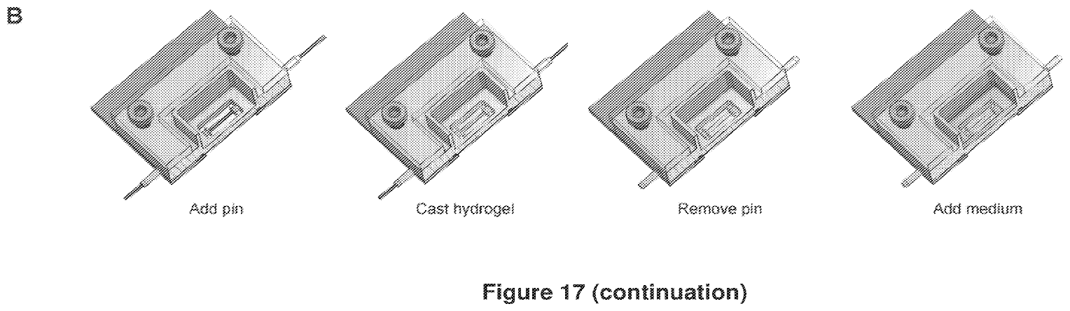

One exemplary individually addressable chip that may be used with the perfusable 3D tubule-on-chip models described herein is shown in FIG. 16. To fabricate this chip, various chip components can be assembled as shown in FIG. 17A. Specifically, a chip may be created by assembling, e.g., an acrylic reinforcement lid with a chip, gasket, glass slide and metal base (FIG. 17A). Once assembled an ECM can be added to an inner reservoir around a pin and crosslinked. The pin may then be removed forming an open channel that can be seeded with cells (FIG. 17B). Medium is then added to the chip reservoir through a removable lid to bathe the ECM. This design allows for easy access to the ECM and medium bathing the ECM.

A microfluidic chip can have multiple microfluidic devices on it. It is the physical platform which houses a microfluidic device, or devices. Microfluidic chips usually range in size from 1 cm to 10 cm, and typically look like a microscope slide. One microfluidic chip can house multiple microfluidic devices.

In certain embodiments, the organoid-derived cells, such as OPTECs and organoid-derived UB cells described herein can be seeded within the multifluidic platform. Specifically, the organoid-derived cells can be seeded within the open channel of the at least one individually addressable, microfluidic chip.

After seeding, cells can form confluent monolayers along channel walls within 3-7 days (FIG. 16C,D,E).

As such, in one embodiment, the multifluidic platform of the described perfusable 3D proximal tubule-on-chip model includes at least one individually addressable chip that includes a first channel consisting of one patent lumen circumscribed by the organoid-derived cells, such as OPTECs and organoid-derived UB cells. In certain embodiments, the first channel is embedded within an ECM. The term “circumscribed” in reference to a “channel consisting of one patent lumen circumscribed by the organoid-derived cells” refers to the channel's lumen being surrounded by the cells. For example, as described herein, the OPTECs circumscribe the patent lumen within the ECM.

The term “embedding” in reference to embedding, e.g., a channel within an extracellular matrix (ECM), refers to either placing the channel(s) on top of the ECM or embedding them within the ECM or printing them into the ECM.

The ECM may be or may include at least one of Matrigel, poly L-lysine, geltrex, gelatin, nitrogen, fibronectin, collagen I, collagen IV, fibrinogen, gelatin methacrylate, fibrin, silk, pegylated gels, collagen methacrylate, basement membrane proteins, or any other biomaterial, or a combination thereof.

In certain embodiment, the ECM comprises at least one of gelatin and fibrinogen.

In certain embodiments, the ECM may comprise 20 mg/mL fibrinogen.

In certain other embodiments, specifically for UB, the ECM may comprise at least one of: HA methacrylate or collagen coated with basement membrane proteins and/or polylysine. In certain embodiments, specifically for UB, the ECM may comprise the 1% (w/v) HA methacrylate, 1.5 mg/mL collagen coated with basement membrane proteins and/or polylysine.

In certain embodiments of the perfusable 3D proximal tubule-on-chip model described herein, the chip may further comprise a second channel.

In certain embodiments, the second channel may be empty (non-seeded).

In certain embodiment, the first channel and the second channel may be co-localized on the chip.

In certain embodiments, the second channel may be embedded within the ECM.

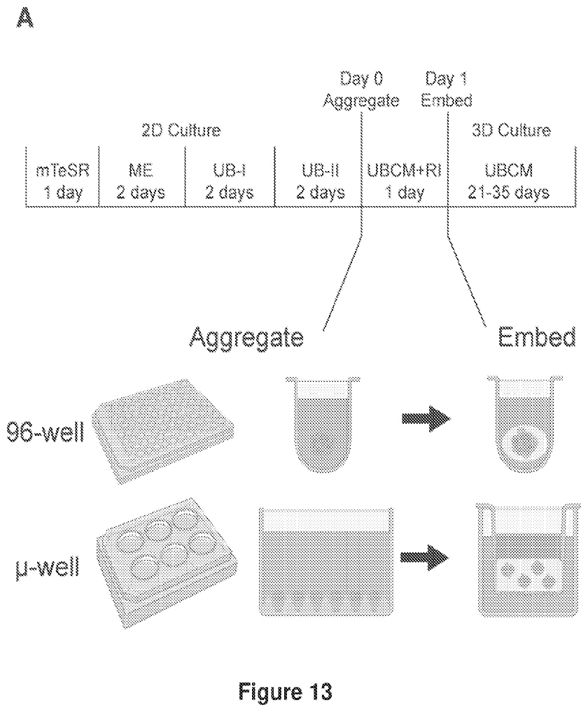

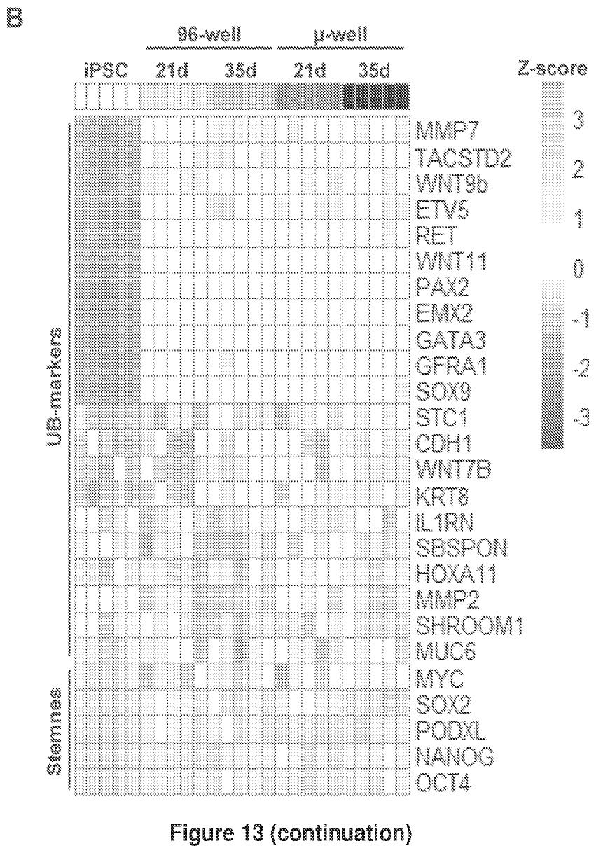

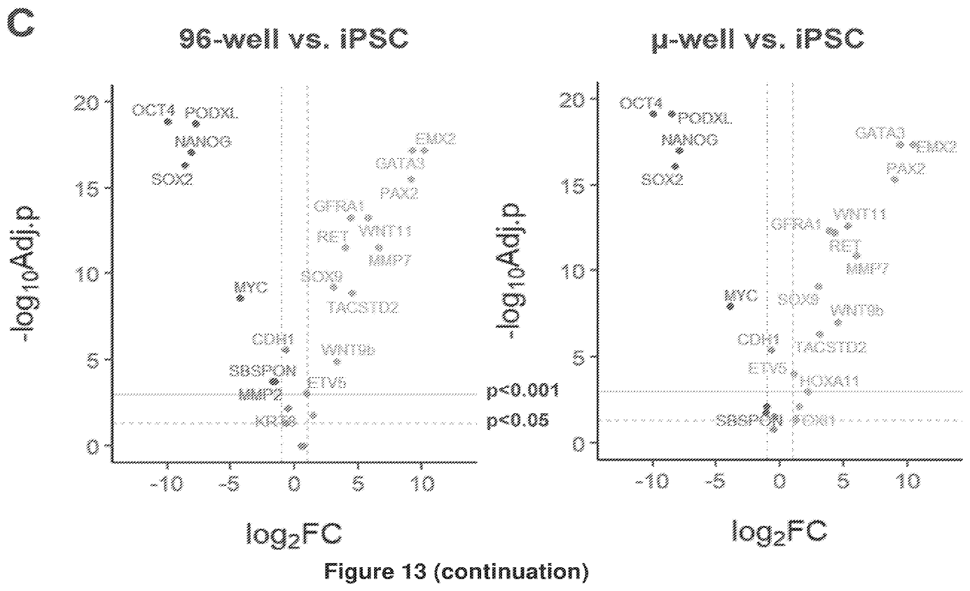

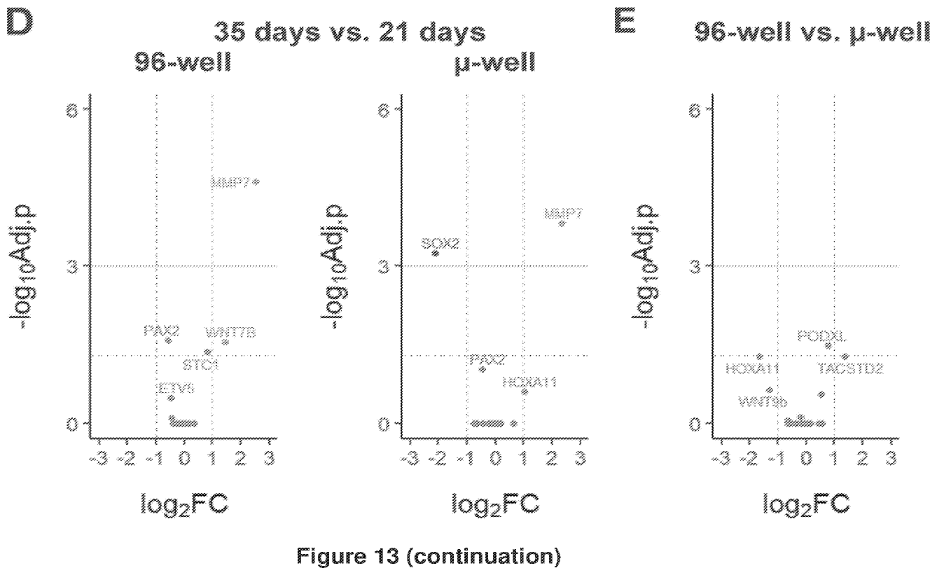

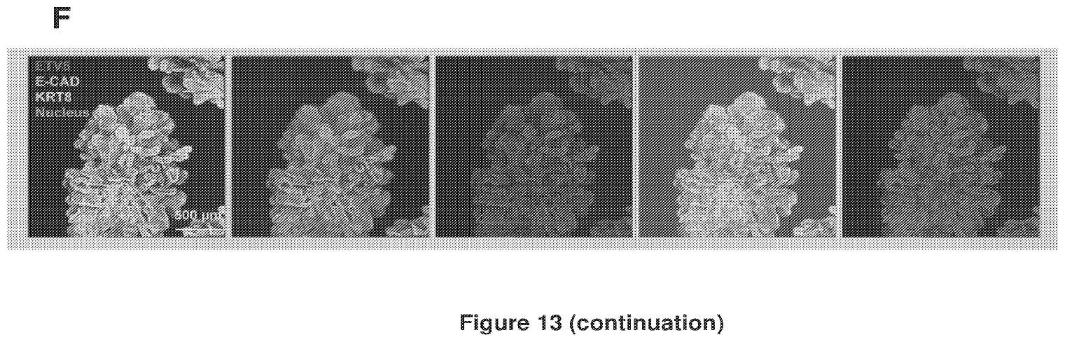

The perfusable chip described herein, in which the ECM surrounding the cast channel(s) can be directly bathed in medium, which may be exposed directly to air (FIG. 13A,B).

In certain alternative embodiments, the multifluidic platform comprises at least two individually addressable chips; alternatively, at least three individually addressable chips; alternatively, at least four individually addressable chips; alternatively, at least five individually addressable chips; alternatively, at least five individually addressable chips; alternatively, at least six individually addressable chips; alternatively, at least seven individually addressable chips; alternatively, at least eight individually addressable chips; alternatively, at least nine individually addressable chips; alternatively, at least ten individually addressable chips; alternatively, from 1 to 6 individually addressable chips; alternatively, from 1 to 10 individually addressable chips; alternatively, from 1 to 100 individually addressable chips; alternatively, from 1 to 500 individually addressable chips.

In certain embodiments, the first channel exhibits a higher cell death response to known nephrotoxins, cisplatin and aristolochic acid, compared to an immortalized proximal tubule epithelial cell line. The term “higher” in this context of cell death means that more of the tubule cells are dying as a result of taking up more of the drugs. As described herein, since the OPTECs have more transporters, they took up more drug than the PTEC-TERTs and hence, died more. This is important since PTEC-TERTs are currently used to predict nephrotoxicity in preclinical settings, however they do a poor job predicting nephrotoxicity, which we attribute to the low drug transporter expression levels.

Certain embodiments relate to a perfusable 3D proximal tubule-on-chip model comprising: (i) OPTECs; and (ii) a multifluidic platform comprising at least one individually addressable chip, wherein the chip comprises a first channel consisting of one patent lumen circumscribed by the OPTECs, and wherein the first channel is embedded within an ECM. The chip may further include a second channel, the second channel being empty (non-seeded), wherein the first channel and the second channel are co-localized on the chip; and wherein the second channel is embedded within the ECM. The OPTECs are isolated from kidney organoids derived from hPSCs. The multifluidic platform can include at least two individually addressable chips. The multifluidic platform can include 6 to 10 individually addressable chips. The ECM may comprise at least one of gelatin and fibrinogen. The ECM may comprise 20 mg/mL fibrinogen. In the perfusable 3D proximal tubule-on-chip model of this embodiment, the OPTECs exhibit: (i) at least 1.5-fold higher drug transporter expression, as compared to an immortalized proximal tubule epithelial cell line; and/or (ii) at least 2-fold higher drug uptake, as compared to an immortalized proximal tubule epithelial cell line. Also, the OPTECs can exhibit a higher expression of basolateral drug transporters OCT2, OAT1, and OAT3, as compared to an immortalized proximal tubule epithelial cell line. In the perfusable 3D proximal tubule-on-chip model of this embodiment, the first channel can exhibit a higher cell death response to known nephrotoxins, cisplatin and aristolochic acid, compared to an immortalized proximal tubule epithelial cell line.

Certain other embodiments relate to a perfusable 3D ureteric bud-on-chip model comprising: (i) organoid-derived UB cells; and (ii) a multifluidic platform comprising at least one individually addressable chip, wherein the chip comprises a first channel consisting of one patent lumen circumscribed by the organoid-derived UB cells, and wherein the first channel is embedded within an ECM. The chip may further comprise a second channel, the second channel being empty (non-seeded), wherein the first channel and the second channel are co-localized on the chip; and wherein the second channel is embedded within the ECM. The organoid-derived UB cells can be isolated from ureteric bud organoids derived from hPSCs. In the perfusable 3D ureteric bud-on-chip model of this embodiment, the multifluidic platform can comprise at least two individually addressable chips; alternatively, the multifluidic platform can comprise 6 to 10 individually addressable chips. In the perfusable 3D ureteric bud-on-chip model of this embodiment, the ECM comprises at least one of gelatin and fibrinogen. The ECM can comprise 20 mg/mL fibrinogen. In the perfusable 3D ureteric bud-on-chip model of this embodiment, the organoid-derived UB cells exhibit epithelial morphology with lateral CDH1, apical BK-alpha and primary cilia, and basolateral NaK⁺-ATPase.

Certain alternative embodiments relate to a perfusable 3D proximal tubule-on-chip model comprising: (i) an OPTEC tubule consisting of one patent lumen circumscribed by organoid-derived proximal tubule epithelial cells (OPTECs); and a multifluidic platform comprising at least one individually addressable chip. The OPTEC tubule may be embedded within an extracellular matrix (ECM). The perfusable 3D proximal tubule-on-chip model can further comprise an unseeded tubule, wherein the OPTEC tubule and the unseeded tubule are co-localized on the chip. The unseeded tubule may also be embedded within the ECM. Different types of suitable ECM were described above. In one embodiment, the ECM can comprise at least one of gelatin and fibrinogen, Alternatively, the ECM can comprise, e.g., 20 mg/mL fibrinogen. In this embodiment, the OPTECs can be isolated from kidney organoids derived from human pluripotent stem cells (hPSCs).

In the described embodiments of the perfusable 3D tubule-on-chip model, the multifluidic platform may comprise at least 2 individually addressable chips; alternatively, at least three individually addressable chips; alternatively, at least four individually addressable chips; alternatively, at least five individually addressable chips; alternatively, at least five individually addressable chips; alternatively, at least six individually addressable chips; alternatively, at least seven individually addressable chips; alternatively, at least eight individually addressable chips; alternatively, at least nine individually addressable chips; alternatively, at least ten individually addressable chips; alternatively, from 1 to 6 individually addressable chips; alternatively, from 1 to 10 individually addressable chips; alternatively, from 1 to 100 individually addressable chips; alternatively, from 1 to 500 individually addressable chips.

Importantly, the studies described herein demonstrated that these UB organoids can be further differentiated to more CD-like cells. Importantly and surprisingly, the ability to perfuse an open channel lined with UB cells was demonstrated, which can then be further differentiated to CD.

### Methods

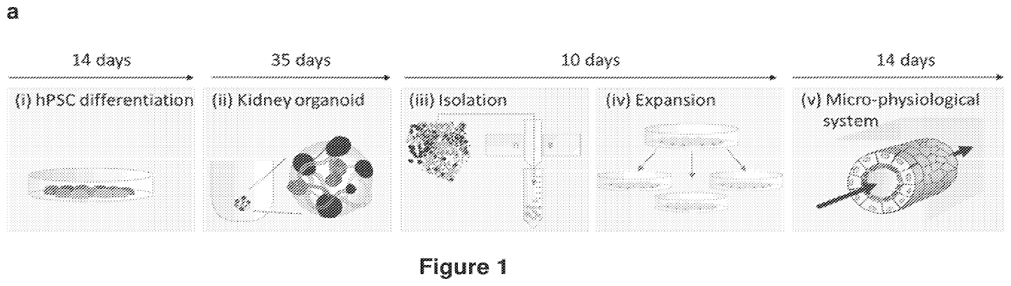

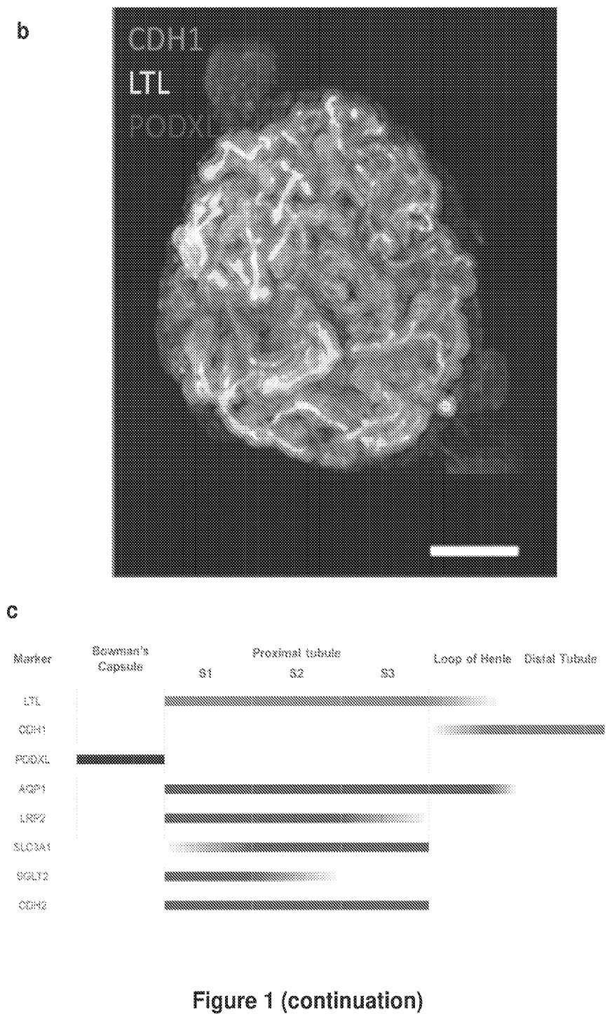

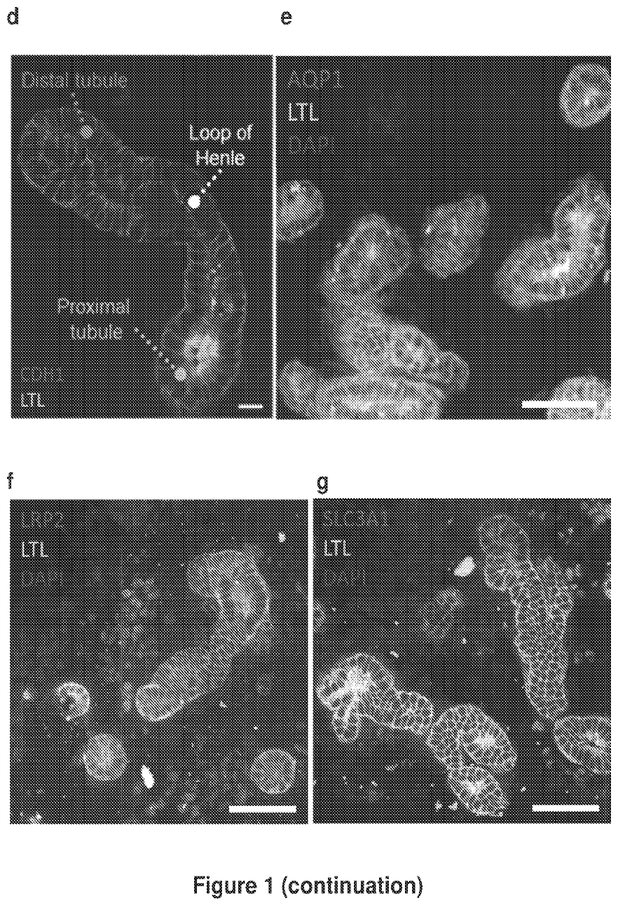

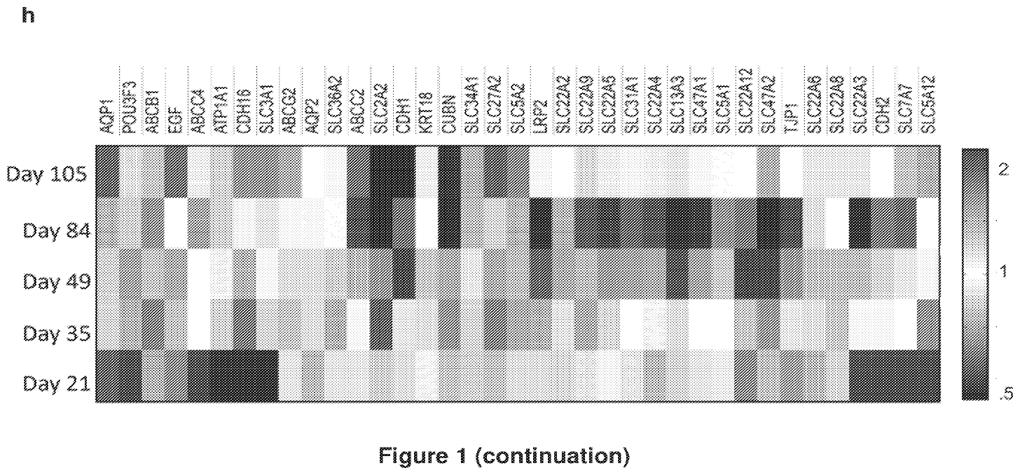

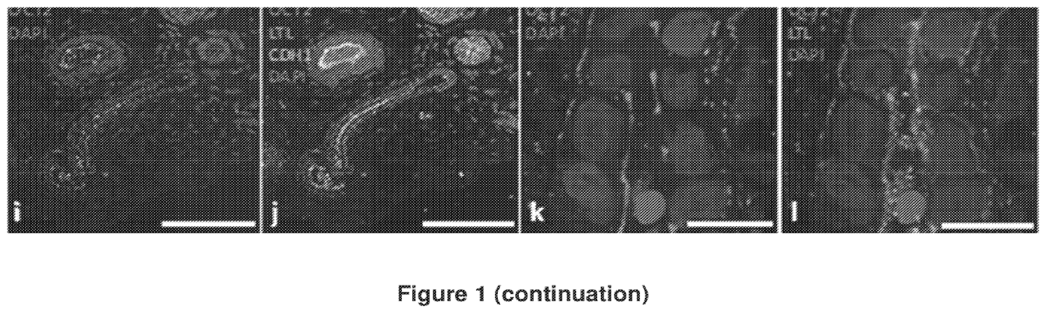

Multiple steps are required to create the perfusable 3D kidney-on-chip models described in detail herein and the steps are provided below. A schematic overview of process used to create 3D organoid-derived tubule-on-chip models is illustrated in FIG. 1a.

The first step of the method of producing a perfusable 3D kidney-on-chip model requires isolating cells from kidney organoids. As discussed above, the organoids, in certain embodiments, can be derived from human pluripotent stem cells (hPSCs).

In certain embodiments, the step of isolating the OPTECs and/or organoid-derived UB cells may be, e.g., by magnetic-activated cell sorting.

In certain embodiments, the organoids used as a source of the OPTECs and/or organoid-derived UB cells may be produced by (i) differentiating hPSCs into nephron progenitor cells; (ii) producing kidney organoids from the nephron progenitor cells; and (iii) maturing the kidney organoids under static culture conditions.

Other methods of producing organoids were previously described in, for example, PCT Publication No. WO 2016/141137A1; PCT Publication No. WO 2022/010901A2; U.S. Pat. Pub. No. 2020/0289709; and U.S. Pat. Pub. No. 2020/0248147A1, which are incorporated herein by reference.

In certain embodiments, the isolated OPTECs are LTL+ OPTECs.

The next step of the method of producing a perfusable 3D kidney-on-chip model requires seeding the isolated the OPTECs and/or organoid-derived UB cells onto a multifluidic platform comprising at least one individually addressable chip.

In certain embodiments, the chip can contain a first channel consisting of one patent lumen, wherein the OPTECs and/or organoid-derived UB cells are seeded within the first channel and circumscribe the first channel.

In certain embodiments, the chip can further comprise a second channel.

In certain embodiments, the second channel may be empty (non-seeded).

In certain embodiments, the chip may be produced by: (i) encapsulating a first channel template within an ECM solution cast into the chip; (ii) enzymatically cross-linking the ECM solution; and (iii) removing the first channel template, thereby forming the first channel, where the first channel can be seeded with the OPTECs and/or organoid-derived UB cells. Optionally, encapsulating a second channel template may be encapsulated within an ECM solution (e.g., gelatin-fibrinogen solution) cast into the chip, the second channel template can be removed and thereby forming the second channel.

In certain embodiments, the first channel may be coated with laminin-511. In certain embodiments, at least one of the first and/or the second channels may be coated with laminin-511. Alternatively, for UB, other basement membrane matrix (i.e., geltrex or Matrigel) and/or polylysine may be used.

The first channel and the second channel may be co-localized on the chip.

In certain embodiments, the first and the second channels can be embedded within the ECM.

The next step of the method of producing a perfusable 3D kidney-on-chip model may further comprise expanding the OPTECs and/or organoid-derived UB cells in 2D culture. A minimum seeding density of the OPTECs and/or organoid-derived UB cells should be in the range 1-10 million cells/mL.

In certain embodiments, described herein are methods of creating a human collecting duct model that can be used directly or embedded within biomanufactured kidney tissues.

### ADDITIONAL EMBODIMENTS

In certain further embodiments, additional types of cells may be incorporated into the perfusable 3D kidney-on-chip model. For example, metanephric mesenchyme derived cells, such as, stromal cells and nephron progenitor cells could be included in the ECM.

The perfusable ureteric bud model developed in this work could be fused with nephron-rich organoids to facilitate function using biofabrication methods. For example, open, perfusable tubes fabricated via existing bioprinting methods, such as those described by Skylar-Scott et al. 2019 or U.S. Pat. Pub. No. 20200289709A1, which are incorporated herein by reference, could be lined with UB cells which then grow and fuse with nephron-rich organoids.

In additional embodiments, the described perfusable 3D kidney-on-chip models may be used to fabricate a UB-lined drainage tube within biomanufactured tissue. For example, in certain embodiments, the described 3D kidney-on-chip models may include a ureter and interconnected network normally generated by UB branching allowing for filtrate drainage.

In certain further embodiments, UB/CD models can be used to model drug delivery/off target effects, such as viral drug delivery through the ureter to target the kidney.

### Uses

In certain embodiments, the perfusable 3D tubule-on-chip models described herein may be used in drug toxicity studies. For example, the perfusable 3D tubule-on-chip models described herein may be used to study renal disease and toxicity. Kidney toxicity is one of the most frequent adverse events reported during drug development. The lack of accurate predictive cell culture models and the unreliability of animal studies have created a need for better approaches to recapitulate kidney function in vitro. Here, described is a perfusable 3D tubule-on-chip that mimics key functions of the human kidney, e.g., kidney proximal tubule.

In certain embodiments, the perfusable 3D tubule-on-chip models described herein may be used in polarized drug uptake studies.

In certain embodiments, the perfusable 3D tubule-on-chip models described herein may be used in personalized drug screening.

In certain embodiments, the perfusable 3D tubule-on-chip models described herein may be used in in disease modeling. For example, the described models may be used to model a kidney disease, such as polycystic kidney disease

In addition, there are a number of developmental disorders and genetic mutations that primarily or specifically impact the collecting ducts (Catena et al., 2007). The most common is polycystic kidney disease, which affects ˜500,000 people in the United States alone and ultimately leads to kidney failure. Other disorders can arise from mutations in transporter proteins, which are associated with a range of symptoms and outcomes (Wagner et al., 2009). These described 3D tubule-on-chip models may be used to model these diseases as well as others.

In certain embodiments, the described patient-specific, functional collecting duct models could accelerate therapeutic target identification. Furthermore, a collecting duct model in vitro could aid in hypothesis testing for mechanistic studies of development, such as investigating the origin and timing of intercalating cell differentiation.

### EXAMPLES

### Example 1

### INTRODUCTION

Three-dimensional, organ-on-chip models that recapitulate kidney tissue are needed for drug screening and disease modeling. Described here is a method for creating a perfusable 3D proximal tubule model composed of epithelial cells isolated from kidney organoids matured under static conditions. These OPTECs are seeded in cylindrical channels fully embedded within an extracellular matrix, where they form a confluent monolayer. A second perfusable channel is placed adjacent to each proximal tubule within these reusable multiplexed chips to mimic basolateral drug transport and uptake.

The described 3D OPTEC-on-chip model exhibits significant upregulation of organic cation (OCT2) and organic anion (OAT1/3) transporters, which leads to improved drug uptake, compared to control chips based on immortalized proximal tubule epithelial cells. Hence, OPTEC tubules exhibit a higher normalized lactate dehydrogenase (LDH) release, when exposed to known nephrotoxins, cisplatin and aristolochic acid, which are diminished upon adding OCT2 and OAT1/3 transport inhibitors. The described integrated multifluidic platform paves the way for personalized kidney-on-chip models for drug screening and disease modeling.

### Methods

### Kidney Organoid Development and Culture

Kidney organoids were prepared using the Morizane protocol²¹. In summary, hPSCs were differentiated to create SIX2⁺ nephron progenitor cells. Upon the formation of the metanephric mesenchyme (day 9 of differentiation), these cells were transferred into low attachment plates to create pre-tubular aggregates and renal vesicles. On day 14, all chemical signaling cues were removed and the kidney organoids were cultured in Advanced RPMI 1640 medium (Thermo Fisher: Gibco, 12633-012) supplemented with 1×GlutaMAX (Thermo Fisher, 35050061). The media changes were performed every 48-72 h through day 49 of static culture by replacing 95 μL of Advanced RPMI+1×GlutaMAX from the wells.

### Isolating Organoid-Derived Proximal Tubule Epithelial Cells

24-well plates were prepared by coating each well with 300 μL of a 1:20 laminin-511 (Biolamina LN511) in DPBS (1×Dulbelco's phosphate buffered saline with calcium and magnesium) solution. The plates were incubated for at least 40 min at 37° C. MACS isolation buffer was prepared by making an ice-cold solution of 1:20 BSA MACS stock solution (Miltenyi Biotec 130-091-376) in MACS rinsing solution (Miltenyi Biotec 130-091-222). Next, d49 organoids were collected from the 96-well plates and transferred into a 15 mL conical tube. The organoids were washed twice with DPBS without calcium and magnesium and incubated in 3 mL of a 0.05% trypsin/EDTA solution for 15 min at 37° C. The organoids were swirled in the solution every 5 min to assist in their dissociation. After 15 min, the organoids were mechanically dissociated by repeated pipetting (20 times over a 40 s period). The pipetting frequency was then increased and carried out an additional 20 times. If needed, the organoids were returned to 37° C. for 5 min in the conical tube and the pipetting steps were repeated until they are fully dissociated.

Once the organoids were fully dissociated, the trypsin/EDTA process is stopped by quenching with 9 mL of ice-cold MACS isolation buffer. Next, the samples were centrifuged in a conical tube at 240 rcf for 4 min. The supernatant was aspirated and the cells were resuspended in 1 mL of MACS isolation buffer. Any residual cell aggregates were removed by sequentially filtering this solution through 70 μm and 40 μm cell strainers. Next, the single cell suspension was centrifuged at 240 rcf for 4 min and resuspend in 80 μL of biotinylated-LTL solution (1:150 B-1325 biotinylated LTL: MACS isolation buffer). The solution was placed on ice for 15 min prior to adding 1.2 mL of MACS isolation buffer and centrifuging at 240 rcf for 4 min. The supernatant was aspirated and the cells were washed with 2 mL of MACS isolation buffer prior to centrifuging again at 240 rcf for 4 min. Upon removing the supernatant, the cells were resuspended in 90 μL of MACS isolation buffer and 10 μL of streptavidin magnetic beads were added to the sample, which was held on ice for 15 min. After 15 min, an additional 1200 μL of MACS isolation buffer was added and the sample was centrifuged at 240 rcf for 4 min, aspirated to remove the supernatant, and washed in 2 mL of MACS isolation buffer. The sample was centrifuged for a final time at 240 rcf for 4 min and resuspended in 500 μL of MACS isolation buffer.

MACS sorting was carried out by placing an MS column (Miltenyi Biotec 130-042-201) into the provided MACS magnet. Before adding the organoid-derived cell solution, 500 μL of MACS isolation buffer was flowed through the magnetic column into a conical tube labeled “LTL-negative”. Next, the cell solution was flowed through the magnetic column, prior to flushing the magnetic column with 500 μL of MACS isolation buffer. Another 500 μL of MACS isolation buffer was added to collect the remaining cells from the conical tube and flowed through the magnetic column. Lastly, the column was flushed with an additional 500 μL of MACS isolation buffer. The magnetic column was removed from the magnet and placed into a fresh 15 mL conical tube labeled “LTL-positive.” 1 mL of MACS isolation buffer was added to the magnetic column and manually pushed through. The LTL⁺ cells were diluted with 5 mL of pre-warmed renal epithelial cell based medium (REGM, Bioscience: Lonza CC-3191), supplemented with 1% fetal bovine serum (FBS). The sample was centrifuged at 240 rcf for 4 min, aspirated to remove the supernatant and resuspended in 1 mL REGM media containing 1% FBS. The number of LTL⁺ cells were counted and seeded onto laminin pre-treated wells at a density of 70,000-100,000 cells/cm². The media was changed every 24 h after isolation to remove apoptotic cells.

### Proximal Tubule Epithelial Cell Culture

Organoid-derived PTECs (OPTECs) were cultured in 24-well plates pretreated with 1:20 laminin-511 (Biolamina LN511) in DPBS with calcium and magnesium. For passages 0 and 1, the OPTECs were cultured in REGM media supplemented with 1% FBS. Upon reaching passage 2, the OPTECs were cultured using LPTEC media supplemented with 1% FBS and 10 μM SB431542 (ab120163). Each passage was performed when the cells reached ˜90% confluency by first washing the cells with DPBS without calcium and magnesium, treating with 0.05% trypsin/EDTA for 3-5 min (or until cells lifted off the plate), followed by quenching with culture medium, centrifuging at 240 ref for 4 min, resuspending in culture media, and seeding each laminin pre-treated well at a density of 70,000-100,000 cells/cm².

For the control chips, we obtained and cultured immortalized human PTECs (PTEC-TERT1 s, ATCC CRL-4031). The culture protocol provided by the manufacturer was followed with one exception. A modified cell media (hereby referred to as LPTEC media) composed of DMEM F-12 without glucose (pH 7.3±0.05), NaHCO3 (1.2 mg/mL), D-glucose (100 mg/dL), ITS (1×concentration, 13146-5ML; Sigma), ascorbic acid (3.5 μg/mL) triiodothyronine (5 μM), PGE1 (25 ng/mL), sodium selenite (3.65 ng/mL), hydrocortisone (25 ng/mL), and EGF (10 ng/mL) was used.

### Extracellular Matrix

An optimized extracellular matrix composed of 20 mg/mL fibrinogen, 1 wt % gelatin, 2.5 mM CaCl₂, and 0.2 wt % transglutaminase in DPBS without calcium and magnesium was used to encapsulate the tubule and basolateral channels within each 3D OPTEC-on-chip model. The fibrinogen was made by first preparing an 80 mg/mL stock solution from lyophilized bovine blood plasma protein (Millipore). It was reconstituted in a controlled manner to prevent agitation by adding sterile DPBS without calcium and magnesium at 37° C. for between 2-3 h. Once complete, the fibrinogen solution was stored in smaller aliquots at −20° C. for later use. A 15% (w/v) gelatin solution (Type A, 300 bloom form porcine skin, Sigma) was prepared by adding prewarmed DPBS without calcium and magnesium to the gelatin powder. This gelatin solution was then stirred for 12 h at 70° C. to allow for complete dissolving of the gelatin. Once completely dissolved, the pH was then adjusted to 7.5 by adding sufficient volume of 1 M NaOH, the gelatin solution was sterile-filtered and stored at 4° C. for later use. A 250 mM CaCl₂) stock solution was made by dissolving CaCl₂ pellets in sterile water and storing at 4° C. The transglutaminase (Moo Gloo, TI) solution was made fresh for each batch of gels by dissolving the powder in DPBS without calcium and magnesium at a concentration of 60 mg/mL. This solution was held at 37° C. for 15 min for complete reconstitution and sterile filtered before use. 500 U/mL stock solutions of thrombin were created by reconstituting lyophilized thrombin (Sigma Aldrich) in sterile water and storing at −20° C. These aliquots were thawed within 15 min before use. After warming to 37° C., these constituents were mixed together in the following order: DPBS without calcium and magnesium, fibrinogen, gelatin, CaCl₂, and transglutaminase. This solution was allowed to equilibrate at 37° C. for 15 min before casting to improve the optical clarity of matrix. The ECM solution was then quickly mixed with a 500 U/mL stock solution of thrombin at a ratio of 250:1 to achieve a desired thrombin of 2 U/mL in the matrix. The thrombin rapidly polymerized fibrinogen into fibrin after the gel was cast into each chip in the MCD, as described below.

### 3D OPTEC-On-Chip Models

The described multiplexed devices include an array of six individually addressable and perfusable 3D OPTECs-on-chip models.

They were fabricated following a multi-step protocol.

First, a channel template composed of fishing line (100-380 μm in diameter) was thread through pins within the polycarbonate, multiplexed chip device (MCD). Unless otherwise noted, all channels were fabricated using the largest fishing line. O-rings (USA Sealing, MCS part #47417118) were then placed on each chip to seal their gel-filled compartments. The device was secured onto a metal base plate with two 50 m×75 mm glass slides using 9 M4 12 mm screws. An enzymatically crosslinked, gelatin-fibrinogen matrix (described above) was then cast into each chip within the device. Next, small screws were used to close the holes used for the casting procedure. Finally, the entire multichip device was placed in a sterile container and allowed to cross-link at 37° C. for 0.5-2 h.

Upon sterilization, a 10 cc syringe barrel (Nordson E F D, 7012112), which served as the media reservoir, was connected to a 0.2 μm syringe filter and 21-gauge syringe nozzle. Next, sterile two-stop Ismatec peristaltic tubing (Cole-Parmer, 95723-12) was attached to the syringe nozzle. Media was then added to the 10 cc reservoir and drawn to the end of the peristaltic tubing. After enzymatically crosslinking the gelatin-fibrin matrix but prior to connecting the tubing to the MCD, the fishing line was gently removed leaving behind two, co-localized empty channels: one of which was seeded with OPTECs to form a 3D proximal tubule and the other that remained unseeded to provide basolateral access for drug uptake studies.

Once the tubing was connected to the MCD, media was flowed through the two channels to ensure all excess gel and air bubbles were removed. Lastly, an adapter composed of peristaltic tubing (2.5 cm in length) was used to connect the outlet pins to silicone tubing that returns media to 10 cc reservoirs. Clamps were used for both the inlet and outlet tubing to prevent undesired pressure changes from accumulating in the channels during media changes and handling.

Before cell seeding, the channels were coated with 40 μL of 1:20 laminin 511 in DPBS with calcium and magnesium solution at 37° C. for at least 45 min. OPTECs were used at passage 3 by first lifting the cells from the 24-well plate, then filtering the cells using a 70 μm cell strainer, resuspending at a density of 10⁶ cells/mL, then seeding 40 μL of the cell solution into the channel. PTEC-TERT1 s (passage 10-20) were used as controls and resuspended at a density of 10⁶ cells/mL in LPTEC media. Both cells were seeded into the channel by removing the silicone tubing from the outlet adapter, unclamping the inlet tubing, and pipetting the cell solution into the empty channel using a p200 pipette.

Both 3D OPTEC- and PTEC-based tubules on chip were cultured using the same protocol.

Before beginning flow through the cell-seeded tubules, the MCDs were placed into the incubator for 4 h. The MCDs were flipped 180° every 15 min during the first hour to ensure even coating of the tubule. After 4 h, perfusion of fresh media was initiated using a peristaltic pump at a rate of 2 μL per minute, equaling to a shear stress ˜0.1 to 0.2 dynes/cm^{I}. The OPTECs typically achieved confluency by day 7, while the PTEC-TERT1 s did so by day 5. Their media was changed every two days, during which a sufficient volume (5 mL per reservoir) of LPTEC media was transferred to ventilated T225 s and incubated for at least 1 h to allow for proper equilibration (37° C. and 5% CO₂). For OPTECs, 1% FBS, 1% aprotinin, 1% anti-anti, and 10 μM SB431542 is added to the media. Once the fresh media was equilibrated, the old media was removed from each reservoir. Lastly, the fresh media was added to each reservoir. To avoid cell aggregation in the OPTEC channels, these chips were flushed via gravity-driven flow at a rate of ˜10 μL/min for 10 min to remove any debris. For PTEC TERTs, 1% FBS, 1% aprotinin, and 1% anti-anti was added to the media.

### Diffusional Permeability Measurements

The barrier function of the 3D OPTECs- and PTEC-TERT1 s based models was assessed by measuring the diffusional permeability of 4.5 kDa inulin. This compound was selected because is neither up taken up nor secreted by PTECs in-vivo. FITC-labeled inulin (Sigma product F3272) was dissolved in prewarmed LPTEC media at a concentration of 100 μg/mL and perfused through the luminal channel at a rate of 20 μL/min for 3 min and 1.5 μL/min thereafter for 15 min. The diffusion of FITC-inulin was calculated by:

$${P}_{d} = \frac{\text{?}}{{I}_{1} - {I}_{b}} \times \frac{{I}_{2} - {I}_{1}}{\text{?}} \times \frac{d}{4}$$

$$\text{?} \text{indicates text missing or illegible when filed}$$

Where P_{d} is the diffusional permeability coefficient, I₁ is the average intensity at the initial time point, I₂ is the average intensity at time t, I_{b} is the background intensity taken before perfusion of FITC-inulin, and dis the diameter of the channel¹³.

### RNA Isolation

RNeasy Mini Kit (Qiagen, 74104) was used for the RNA isolation protocol. To begin lysis of the tubules, the inlet tubing was cut, disconnected from the reservoir, and placed into a sterile 1.6 mL Eppendorf tube. Next, the outlet tubing was disconnected from the chip and 300 μL of RLT buffer was pipetted through each tubule and the perfusate was collected using an Eppendorf tube. After this step, the manufacturer's instructions were followed to isolate the RNA from the collected lysate. Briefly, 70% EtOH was added to the lysate and the solution was transferred to the spin column. A series of washing steps were carried out using the provided RW1 and RPE buffers, sequentially. Finally, the RNA was isolated using RNase-free water and immediately stored at −80° C.

### Nanostring Gene Expression Analysis

Nanostring analysis was carried on samples obtained from RNA isolation at the Boston Children's Hospital IDDRC Molecular Genetics Core Facility. First, a hybridization master mix was created by adding 70 μL nCounter Sprint hybridization buffer to a Reporter CodeSet. Next, hybridization reactions were set up by mixing 8 μL of the master mix with 5 μL of the RNA isolate, and 2 μL of the capture probe set. The reactions were then placed into a thermocycler and run overnight. The next morning, the hybridization reactions were spun down and brought to 35 μL total volume by adding 20 μL of RNase-free water. The 35 μL of sample was then loaded into a SPRINT cartridge and run using a Nanostring nCounter SPRINT profiler. The resulting data was analyzed using the nSolver4.0 software. Before comparing any sample groups, all samples were normalized to positive controls as well as housekeeping genes: ACTB, GAPDH, and TBP. Ratios comparing the sample groups were calculated in nSolver4.0 and values were plotted using GraphPad Prism 9.

### Nephrotoxicity Testing

Before beginning these experiments, three important steps were carried out: (1) all reservoirs were supplied with fresh media, (2) all outlet tubing was cut to the same length, and (3) all chips were subjected to one-way rather than recirculating back to the media reservoir. After each chip within the MCDs was subjected to one-way flow (2 μL/min) for 4 h, a baseline sample of the perfusate was collected from the luminal outlet tubing for 3 h into a 1.6 mL Eppendorf tube. Next, cisplatin (50 μM) or aristolochic acid (25 μM) was delivered basolaterally on chip by manually pipetting each drug solution to the appropriate basal media reservoir. Next, all tubing was reconnected to the peristaltic pump and allowed to flow at a flow rate of 2 μL/min. After 48 h, another sample of the perfusate was collected from the luminal outlet tubing for 3 h into a 1.6 mL Eppendorf tube. All collected luminal media samples were spun down at 5000 rcf for 5 min to remove cellular debris. Each supernatant was transferred to a fresh tube and stored at −20° C. to run the LDH assay. For samples pre-treated with inhibitors, cimetidine (1 mM) or probenecid (2 mM) was first added to the basal reservoir, delivered basolaterally, and allowed to diffuse through the gelatin-fibrin matrix during recirculating flow for 1 h.

### Ldh Analysis of Media Perfusate

LDH assays were performed according to the manufacturer's instructions (Promega, CytoTox Non-Radioactive Cytotoxicity Assay, G1780). The frozen media samples were thawed at room temperature and used after this first thaw cycle. Next, 50 μL of each media was transferred to a 96-well plate and incubated at 37° C. for 30 min with 50 μL of CytoTox 96 reagent. 50 μL of stop solution was then added, all bubbles were removed using a syringe, and their absorbance was recorded at 490 nm using a plate reader.

Normalized LDH release was calculated using the following equation:

$$Normalized LDH release = \frac{LDH \text{?} - {LDS}_{baseline}}{{LDS}_{baseline}} \times 100$$

$$\text{?} \text{indicates text missing or illegible when filed}$$

LDH₄₈ was the recorded absorbance of the 48-h perfusate sample and LDH_{baseline} was the recorded absorbance of baseline perfusate sample before drug exposure. Normalized LDH release was calculated for each of the tubules individually and plotted into sample groups comparing OPTEC-on-chip and PTEC-TERT1-on-chip models subjected to each drug ±inhibitor.

### Imaging and Immunostaining

We used an inverted Leica DM IL microscope to carry out all phase contrast microscopy (scope objectives ranging from 1.25× to 40×). Images were taken while the tubules are still in culture. For confocal microscopy, all images were taken of fixed and stained samples. To fix the chips, tubules were flushed with DPBS containing calcium and magnesium for 10 min under gravity-driven flow. Next, 10% buffered formalin was flowed through the tubular channel for 10 min. The inlet and outlet tubing were then disconnected from each chip, the MCD was removed from the baseplate, and the gels along with the embedded tubules were cut out. These samples were placed into labeled 15 mL conical tubes containing 10% buffered formalin and allowed to continue fixing for 10 min. The buffered formalin was washed away by 3 washes in DPBS with calcium and magnesium. Samples were then sliced and placed overnight in a blocking solution containing 1 wt % donkey serum and 0.125% Triton X-100 in DPBS with calcium and magnesium. The blocking solution was washed away by 3 washes of DPBS. Primary antibodies were added for 24 h at 4° C. in a staining solution containing 0.5 wt % BSA and 0.125% Triton X-100 in DPBS with calcium and magnesium. Primary antibodies were washed away by 3 washes of DPBS, with the last wash being applied overnight to remove excess, unbound primary antibody. Secondary antibodies were then incubated overnight at 4° C. in the same staining solution used for the primary antibodies. Samples were then stained with DAPI and washed for 2 h in DPBS before imaging. Antibody lists for kidney organoid and tubule staining were provided Tables 1 and 3 below, respectively.

TABLE 1
Immunostaining reagents for organoid staining.

| Antibody for organoid sections | Manufacturer | Catalog # |
|---|---|---|
| CDH1 | Abcam | ab11512 |
| LTL | VectorLabs | B-1325 |
| PODXL | R&D | AF1658 |
| AQP1 | Abcam | ab2219 |
| LRP2 | Sigma Aldrich | MABS489 |
| SLC3A1 | Sigma Aldrich | HPA038360 |
| OCT2 | NSJ Bioreagents | F43298 |
| OAT1 | Biorbyt | orb11177 |
| SGLT2 | Abcam | ab8562 |

TABLE 2
Immunostaining reagents for tubule staining

| Antibody or | | | Host | |
|---|---|---|---|---|
| Stain for | | Catalog | Species & | Concen- |
| tubules | Manufacturer | # | Reactivity | tration |
| Na/K | Abcam | ab76020 | Rabbit anti- | 1:300 |
| ATPase | | | human | |
| LTL | Vector Lab | B-1325 | N/A | 1:200 |
| OCT2 | Abcam | ab170871 | Rabbit anti- | 1:200 |
| | | | human | |
| AQP1 | Santa Cruz | SC25287 | Mouse anti- | 1:300 |
| | | | human | |
| Laminin | Abcam | ab11575 | Rabbit anti- | 1:300 |
| | | | human | |
| Col IV | Abcam | ab6586 | Rabbit anti- | 1:300 |
| | | | human | |
| Acetylated | Abcam | ab24610 | Mouse anti- | 1:300 |
| alpha tubulin | | | human | |
| ActinRed | Life | R37112 | N/A | 2 drops per mL |
| | Technologies | | | |
| ActinGreen | Life | R37110 | N/A | 2 drops per mL |
| | Technologies | | | |

For confocal images, an upright Zeiss LSM 710 with water-immersion scope objectives (ranging from 10 to 40×) was used with spectral lasers at 405, 488, 514, 561, and 633 nm. ImageJ software was used to reconstruct all confocal images.

### Electron Microscopy

For transmission electron microscopy (TEM), OPTECs were fixed using 2.5% glutaraldehyde, 1.25% paraformaldehyde, and 0.03% picric acid in 0.1 M sodium cacodylate buffer (pH 7.4) for a minimum of several hours. Small samples (1 mm×1 mm) were removed and washed in 0.1 M cacodylate buffer and bathed in 1% osmiumtetroxide (OSO₄) (EMS) and 1.5% potassiumferrocyanide (KFeCN₆) (Sigma) for 1 h, washed in water 3× and incubated in 1% aqueous uranyl acetate (EMS) for 1 h followed by 2 washes in water and subsequent dehydration in varying grades of alcohol (10 min each; 50%, 70%, 90%, 2×10 min 100%). The samples were then put in propyleneoxide (EMS) for 1 h and incubated overnight in a 1:1 mixture of propyleneoxide and TAAB Epon (Marivac Canada Inc. St. Laurent, Canada). The following day the samples were embedded in TAAB Epon and polymerized at 60° C. for 48 h. Ultrathin sections (about 60 nm) were cut on a Reichert Ultracut-S microtome, placed on copper grids stained with lead citrate and examined in a JEOL 1200EX Transmission electron microscope and images were recorded with an AMT 2 k CCD camera. Image analysis was performed using ImageJ software.

For scanning electron microscopy (SEM), perfused OPTECs in 3D were fixed using 10% buffered formalin for 1 h. The samples were thinly sliced (˜1 mm thick) to expose cells circumscribing the open lumen. The fixative was washed away using PBSx2 and subsequent dehydration in varying grades of ethanol (20 min each; 30%, 50%, 70%, 90%, 3×20 min 100%). The samples were then placed in 50% ethanol and 50% hexamethyldisilazane (HMDS) for 30 min followed by 100% HMDS 3×30 min. All steps were performed in a closed and sealed glass container. After the final washing with HMDS, the samples were removed and placed in an open container under N₂ in the fume hood to dry. Dried samples were mounted to aluminum pin mounts using conductive carbon tape, sputter coated with gold, and imaged with a Tescan Vega SEM.

### Results

### 3D Organoid-Derived Proximal Tubular Epithelial Cells-On-Chip Models

Multiple steps were required to create our 3D OPTECs-on-chip models: (1) differentiate hPSCs into nephron progenitor cells, (2) produce and mature kidney organoids under static culture, (3) isolate OPTECs by magnetic-activated cell sorting (MACS)^{21,22}, (4) expand these OPTECs in 2D culture, and (5) seed these OPTECs in channels embedded within a gelatin-fibrin ECM on each chip within our integrated multifluidic platform (FIG. 1a).

Following the Morizane protocol²¹, the hPSCs were first differentiated and assembled into multicellular aggregates composed of nephron progenitor cells (day 9 of differentiation). Upon further differentiation (days 14-49), nephron-rich kidney organoids developed that expressed glomerular (podacalyxin-like protein 1, PODXL), proximal tubule (lotus tetragonolubus lectin, LTL), loop of Henle, and distal tubule (cadherin 1, CDH1) markers (FIG. 1b)²¹.

To identify the optimal marker for isolating OPTECs from this multicellular milieu, LTL, aquaporin 1 (AQP1), LDL receptor related protein 2 (LRP2), solute carrier family 3 member 1 (SLC3A1), and sodium-glucose cotransport 2 (SGLT2) were evaluated. Since LTL was expressed in three segments of the proximal tubule, this marker allowed for greater cell yield during isolation. This marker also had less overlap with the loop of Henle, making it a better candidate compared to AQP1 (FIG. 1c-g). MACS sorting was used to isolate LTL⁺ cells, which is more benign than fluorescence-activated cell sorting (FACS) via flow cytometry.

To determine optimal time for kidney organoid maturation under static culture conditions before OPTEC isolation, transporter expression was evaluated in freshly isolated LTL⁺ cells using a Nanostring assay. A single-cell transcriptomics study carried out previously, which compared the maturity of human kidney organoids produced from two leading protocols^{21,23}, showed that kidney organoids (day 26) were reflective of less mature renal tissues²⁴, resembling first trimester kidneys²⁵. Hence, LTL⁺ cells were isolated from kidney organoids matured under static culture conditions at different time points between days 21 and 105. During this period, the average transporter expression was highest at day 49 (FIG. 1h). However, it was found that the organic cation transporter 2 (OCT2) in kidney organoids (day 49) was not properly polarized to the basal side (FIG. 1i-l), as would be expected in vivo. While kidney organoids have been shown to elicit injury responses when exposed to known nephrotoxins, their tubules lack proper polarity when cultured under static conditions.

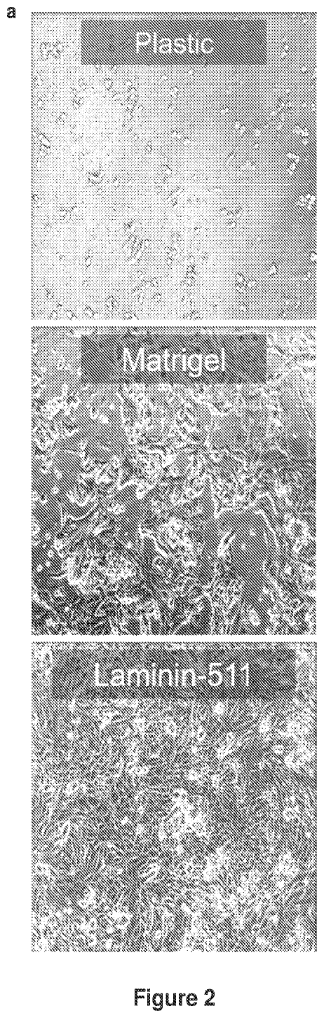

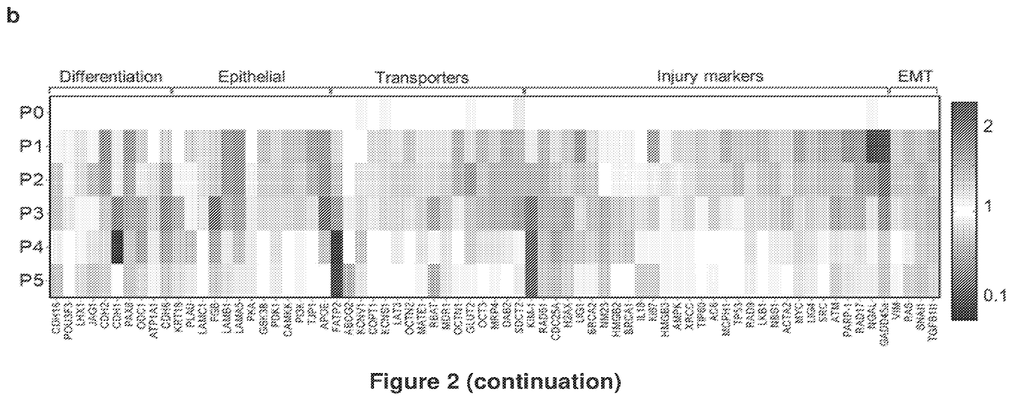

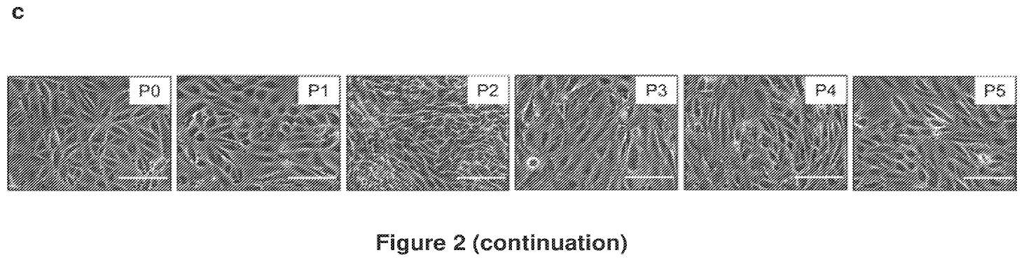

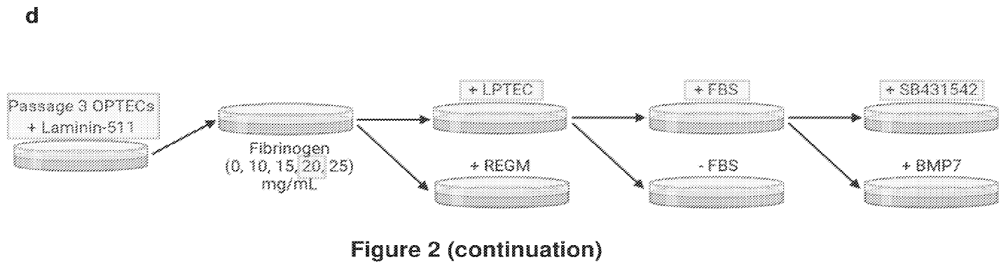

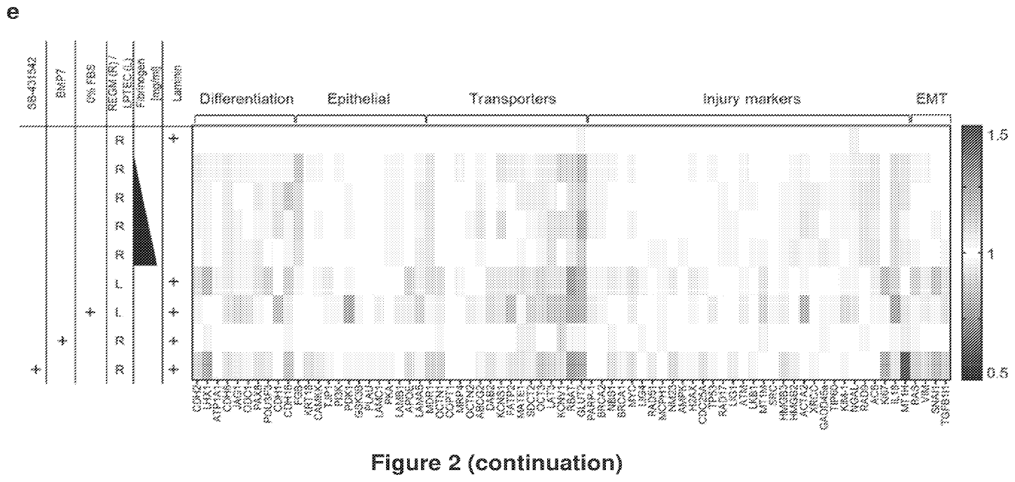

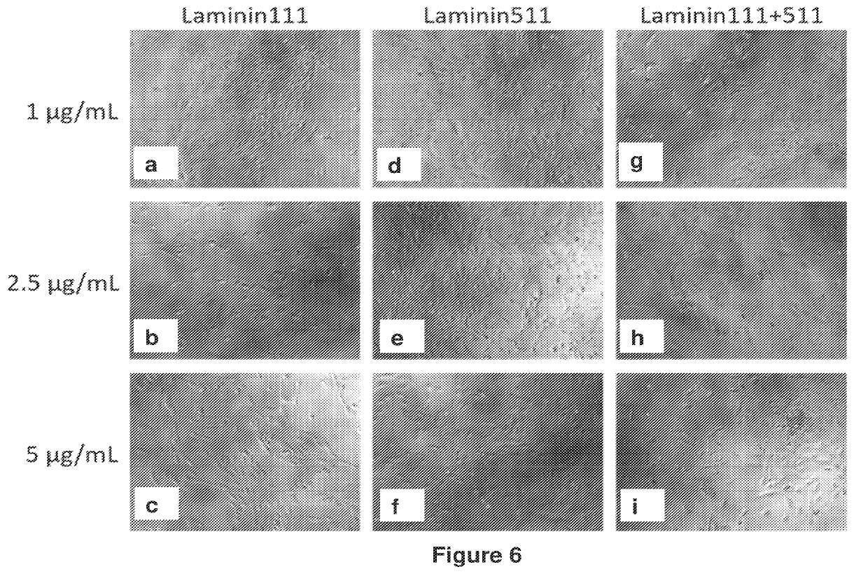

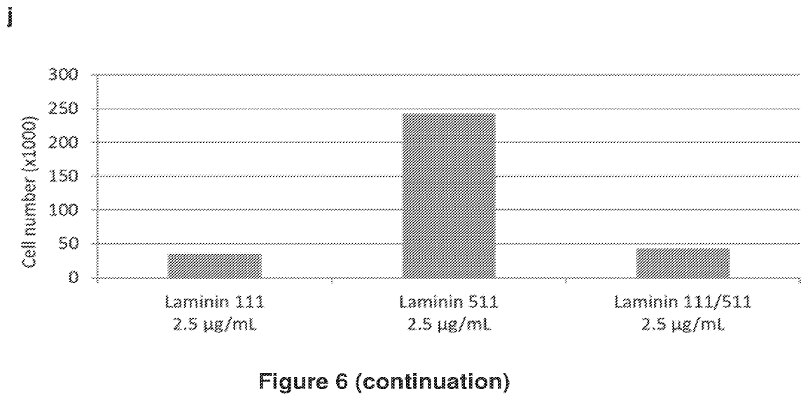

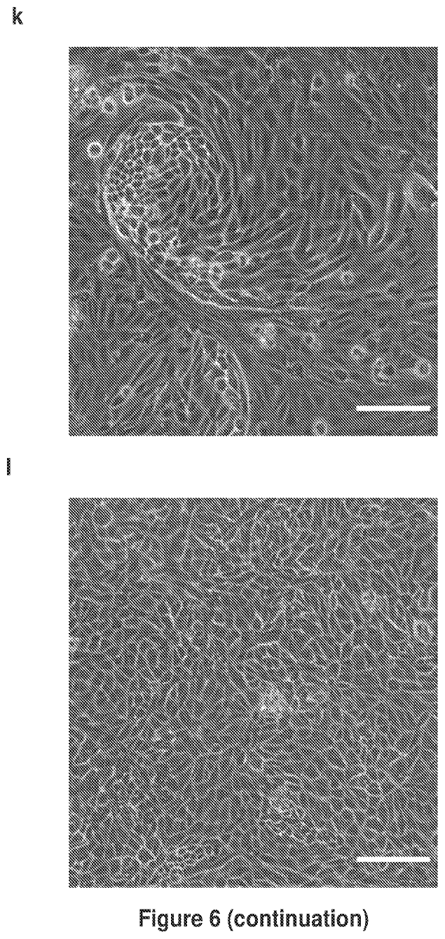

The next step to constructing the 3D OPTEC-on-chip model was to expand the isolated LTL⁺ cells in 2D culture. OPTECs were plated on pure plastic, matrigel-coated, and different laminin-coated substrates to determine which of these substrates is most conducive to cell growth (FIG. 2a). It was found that laminin-511 coated substrates yielded the fastest growth rates and resulted in OPTECs that closely mimicked the cuboidal phenotype of cultured immortalized proximal tubule cells (FIG. 6). Hence, OPTECs were cultured on laminin-511 coated substrates up to five passages, where passage 0 denoted LTL⁺ cells freshly isolated from kidney organoids, to determine the optimal passage for seeding into 3D tubules. Several factors were taken into consideration, including their differentiation, epithelial, transporter, injury, and dedifferentiation markers (FIG. 2b) and cell morphology (FIG. 2c). Both the epithelial and transporter marker expression were highest for OPTECs at passage 3, which indicated that these cells should be seeded in the 3D tubules following their second passage. Brightfield images of the OPTECs between passage 0 and 5 revealed that these cells exhibit proper cuboidal morphology through passage 5.

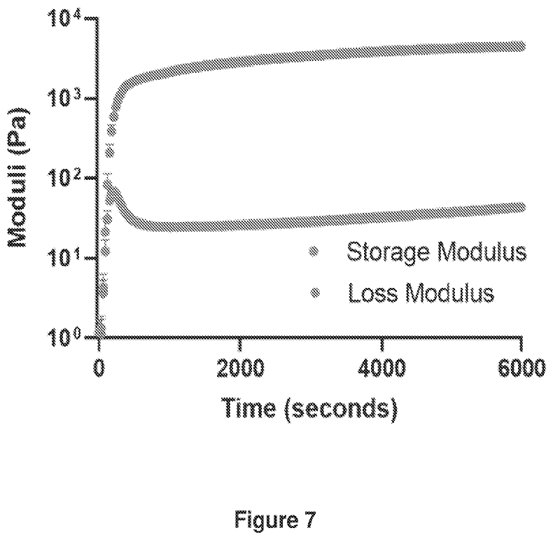

Finally, the effects of different ECM, media, and media supplement conditions on OPTECs cultured in 2D (FIG. 2d, e) were evaluated. In prior work, the current inventors showed that PTEC-TERT1 s grow best on an extracellular matrix composed of 25 mg/mL fibrinogen, 1 wt % gelatin, 2.5 mM CaCI₂, and 0.2 wt % transglutaminase in DPBS media without calcium and magnesium¹⁴. Therefore, OPTECs (passage 3) were cultured on ECMs pre-treated with laminin-511 that contained varying concentrations of 0, 10, 15, 20, and 25 mg/mL fibrinogen at a fixed gelatin content of 1 wt %. It was observed that the optimal transporter expression occurred for OPTECs cultured on an ECM with 20 mg/mL fibrinogen. The optimized ECM exhibited a measured shear elastic modulus, G′, of ˜4400 Pa, akin to the cortex of a healthy kidney (stiffness of ˜4000 Pa)¹³ (FIG. 7).

Next, the effects of two different media were tested: (1) modified DMEM F-12 medium described by previous work¹⁴ (LPTEC) and (2) commercially available renal epithelial growth medium (REGM). The OPTECs exhibited higher epithelial marker expression, higher transporter expression, and lower injury marker expression when cultured in LPTEC akin to prior observations for PTEC-TERT1 s¹⁴. To further promote cell attachment, growth, and proliferation, 1% FBS was added to the media. To minimize epithelial to mesenchymal transition (EMT), the effects of transforming growth factor (TGF-β), bone morphogenic protein 7 (BMP7) and TGF-β/activin/NODAL pathway (SB431542) inhibitors were explored (FIG. 6k-l)²⁶. The latter inhibitor led to a down regulation of EMT markers as well as improved OPTEC phenotype in 2D cell monolayers, increasing the amount of time these cells could be maintained in culture. After optimizing these parameters, OPTECs with proper cuboidal phenotype grew at rates similar to those observed for PTEC-TERT1 s in standard culture.

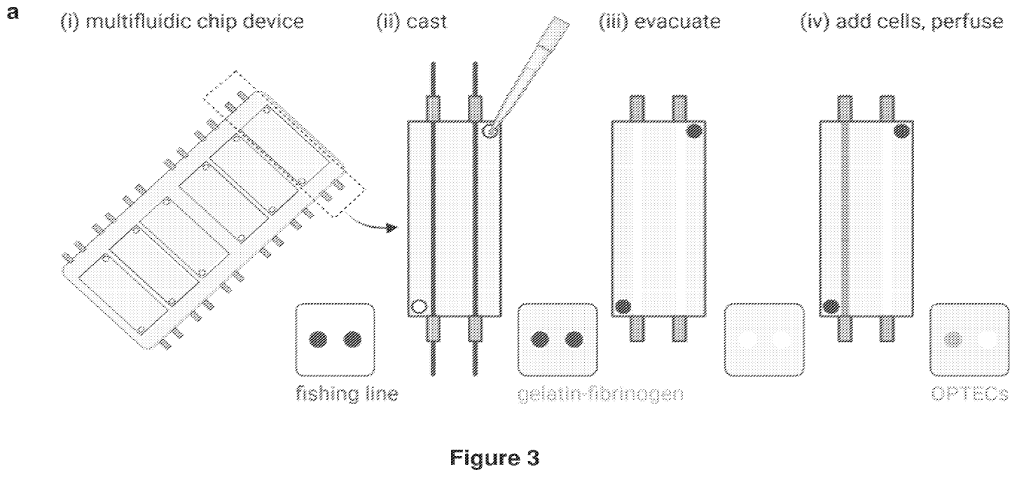

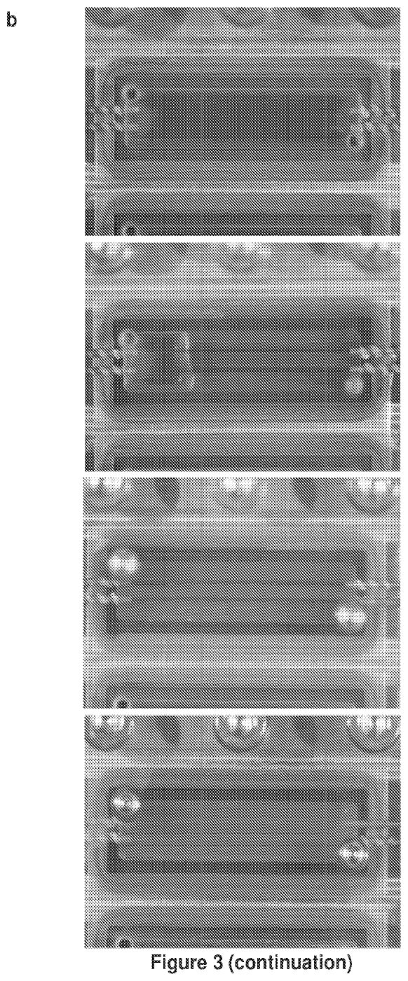

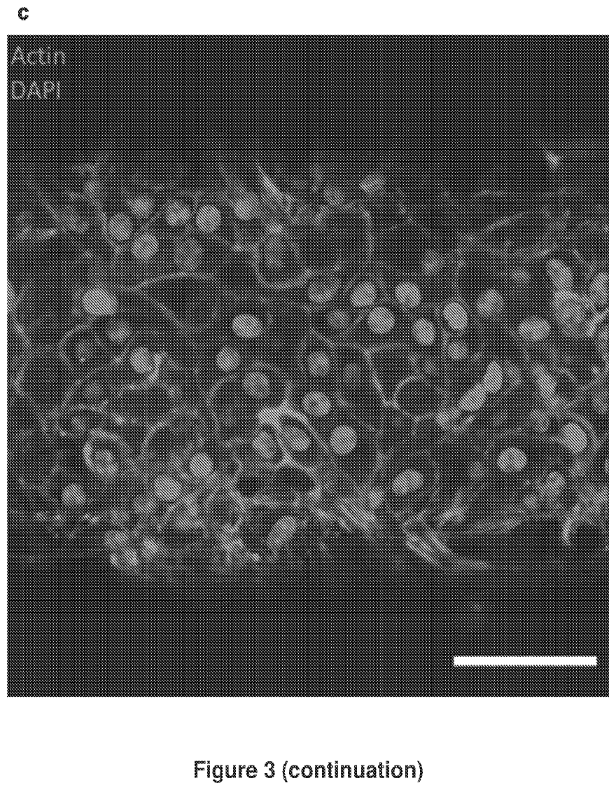

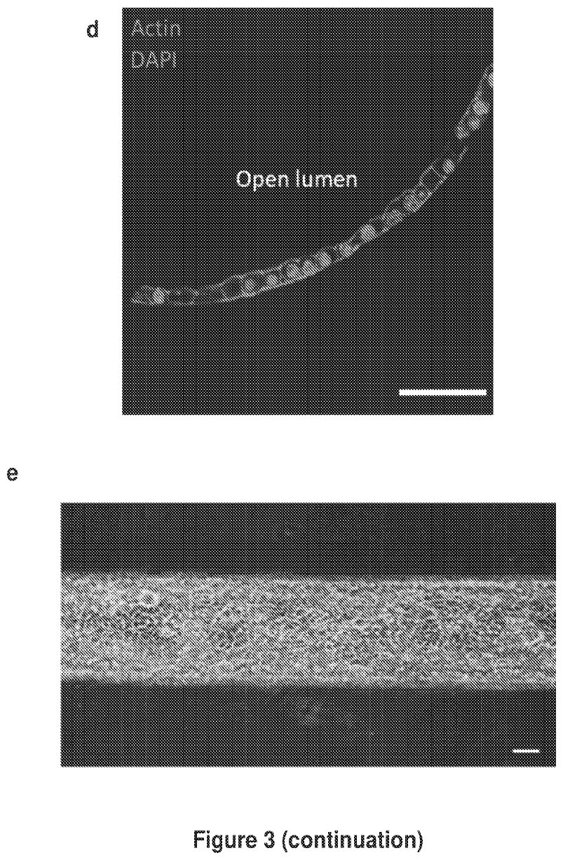

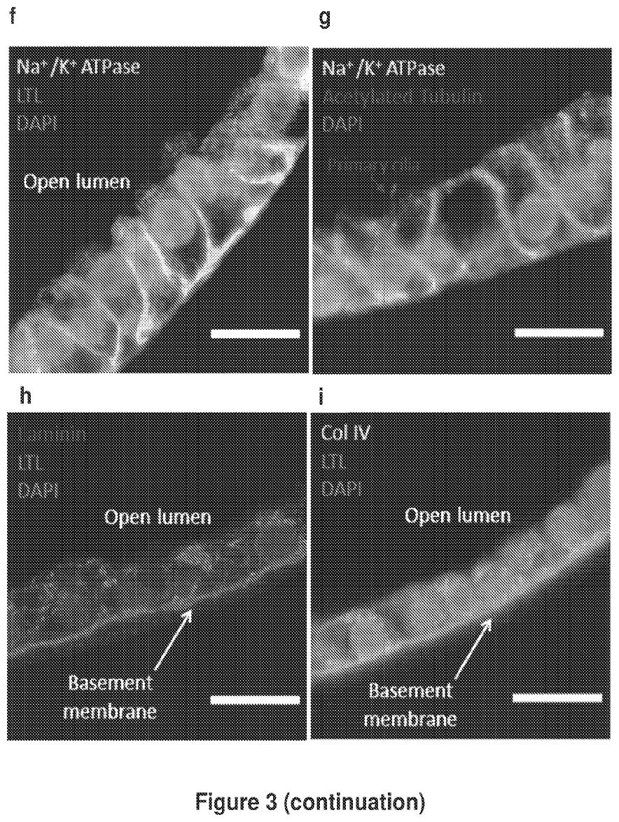

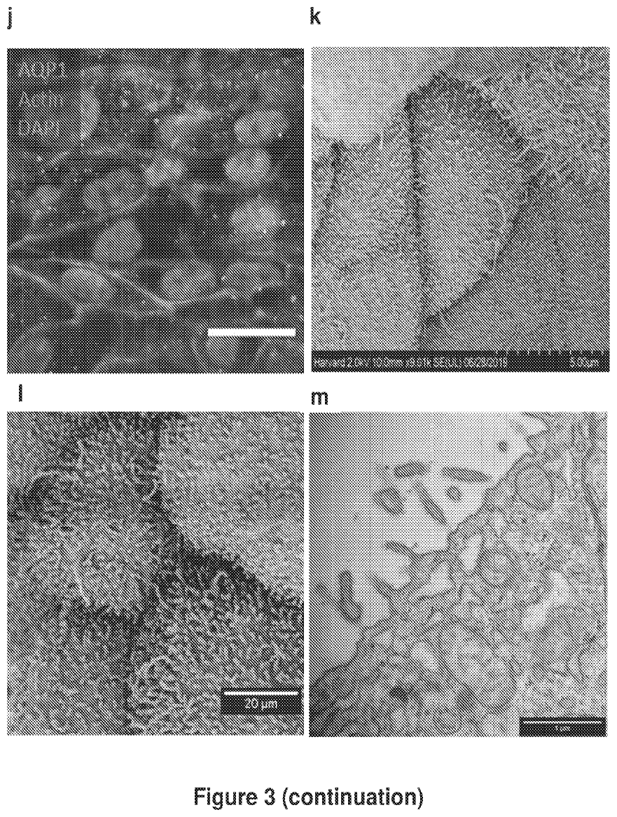

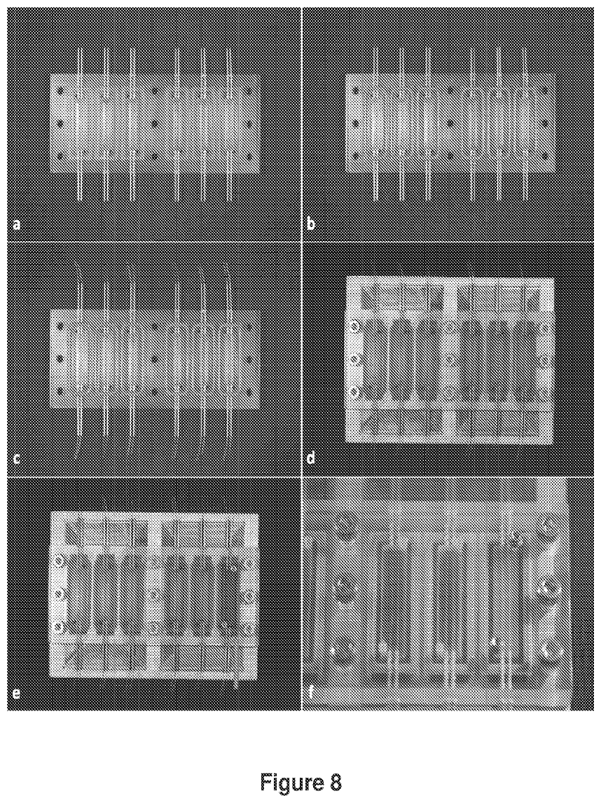

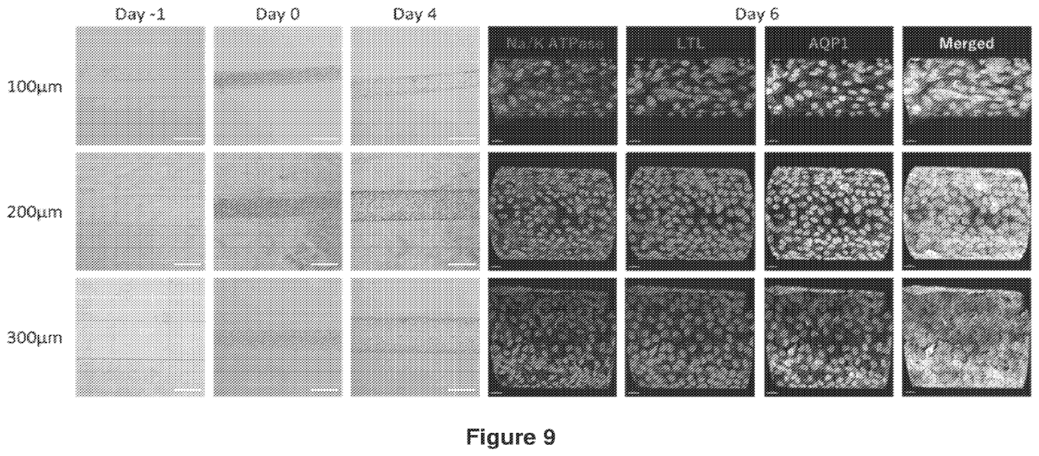

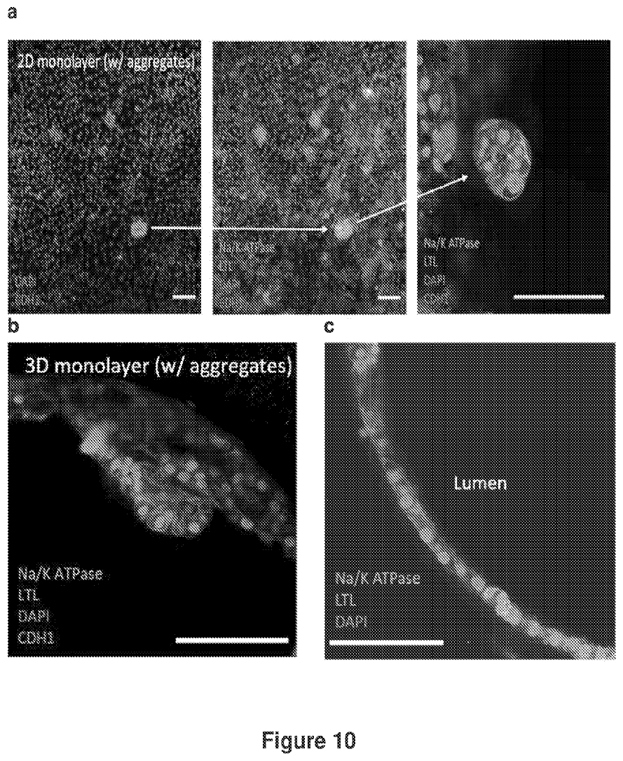

The final step was to create an integrated multiplexed platform composed of six, individually addressable and perfusable 3D OPTEC-on-chip models. Inventors' original convoluted 3D proximal tubule-on-chip model was produced by multi-material bioprinting^{13,14}. Since many labs lack access to this sophisticated equipment, we designed this reusable multiplexed platform using a modified pin pullout method (FIG. 8). Briefly, fishing line, which served as a channel template, was encapsulated within a gelatin-fibrinogen solution that is cast into each chip. Once the matrix was enzymatically cross-linked, the fishing line was removed leaving behind open tubules that can be seeded with OPTECs (FIG. 3a, b). A minimum seeding density of 10 million cells/mL was required to consistently obtain confluent tubules within a 7-day period. Importantly, it was noted that it was possible to create proximal tubules with diameters as small as 100 μm by this method (FIG. 9). However, seeding those channels at the requisite seeding density was challenging, so chips with a tubule size of 380 μm in diameter were used. To avoid cellular aggregates, the OPTECs were filtered through a 70 μm cell strainer prior to the seeding process. To maintain a confluent epithelial layer after day 7, tubules were flushed intermittently during each media change under gravitational flow (˜10 μL/min) for 10 min. The procedure greatly reduced the number of cellular aggregates and allowed the tubules to be cultured for several weeks post-confluency. In some instances, a small population of cellular aggregates composed of LTL⁺ and CDH1⁺ cells remained within the tubules (FIG. 10), which may arise due to self-assembly.

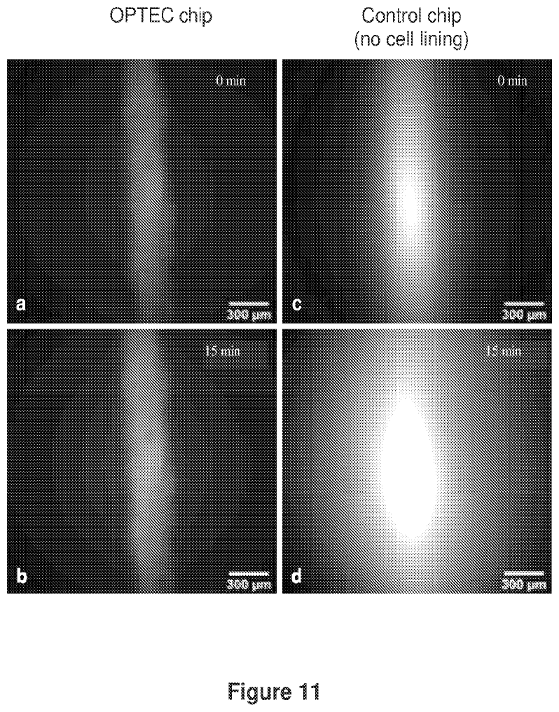

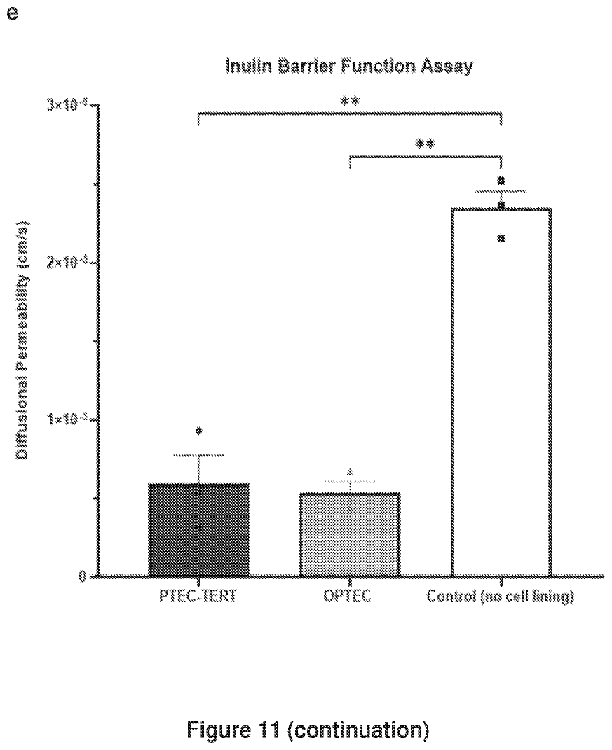

However, most tubules exhibited the desired cuboidal phenotype and assembled into a confluent epithelial monolayer in which OPTECs circumscribe the lumen (FIG. 3c, d). A combination of light microscopy, scanning electron microscopy (SEM), and transmission electron microscopy (TEM) was used to characterize OPTEC tubules after 14 days of perfusion on chip. Phase-contrast microscopy revealed that OPTECs grow throughout the tubule packing together in a columnar fashion (FIG. 3e). The expression of the epithelial markers LTL+ and Na+/K+ ATPase and their appropriate apical and basolateral localization were confirmed, respectively (FIG. 3f) by immunofluorescence (IF) imaging, akin to the in vivo phenotype. Primary cilia were also observed by staining for acetylated tubulin (FIG. 3g). The OPTECs deposited basement membrane proteins, laminin (FIG. 3h) and collagen IV (Col IV, FIG. 3i). The proximal tubule-specific water channel was also observed throughout the tubule, as revealed by the speckled pattern on their membrane surface arising from AQP1 staining (FIG. 3j). SEM images of the apical side of the OPTEC tubule revealed the formation of a confluent epithelium and the presence of primary cilia (one per cell) as well as a pronounced brush border (FIG. 3k, l). Using TEM, a higher magnification view was obtained that showed individual microvilli that protrude from the apical surface of the OPTECs as well as tight junctions between adjacent cells (FIG. 3m). Finally, FITC-inulin was perfused through the OPTEC tubules to assess their barrier function. Importantly, it was found that their diffusional permeability was comparable to that observed for PTEC-TERT1 tubules (control)¹³ (FIG. 11). Based on the above data, the described 3D OPTEC-on-chip model appears well suited for polarized drug uptake and toxicity studies.

### Improved Expression and Polarization of Drug Transporters

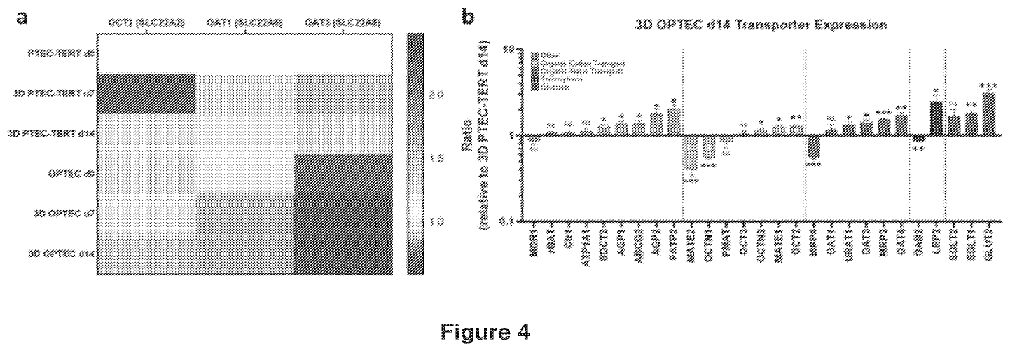

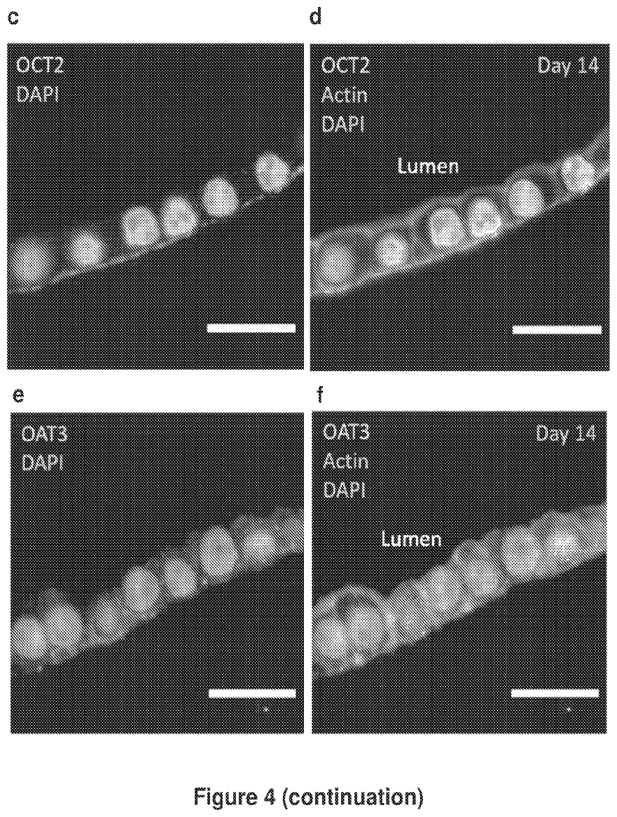

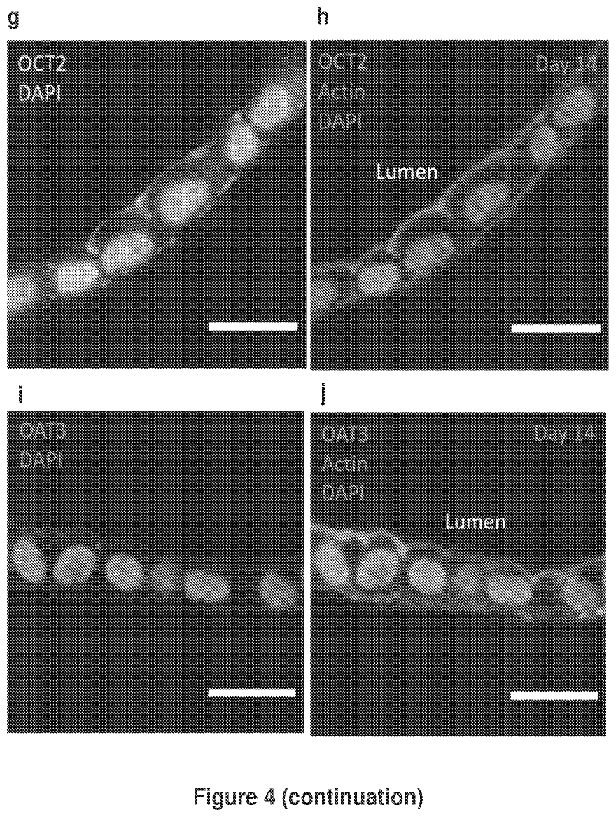

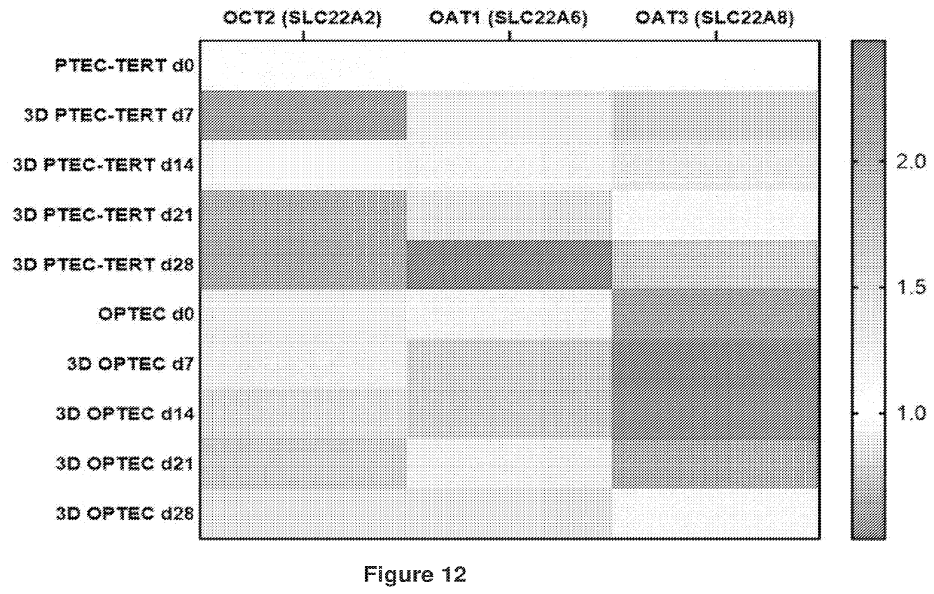

Known nephrotoxins, such as cisplatin and aristolochic acid, were transported basolaterally through organic cation (OCT2) and anion (OAT1/3) transporters, respectively^{27,28}. To determine whether our 3D OPTEC-on-chip model exhibits proper expression and polarization of these key drug transporters, the expression levels of OCT2, OAT1, and OAT3 within the OPTEC tubules was measured over a 28-day period of perfusion using Nanostring analysis. We compared the expression of OCT2, OAT1, and OAT3 observed for OPTECs-on-chip to control chips composed of PTEC-TERT1 s under the following conditions: immediately upon seeding (day 0), after the cells achieve confluency (day 7) followed by one week (day 14), two weeks (day 21), and three weeks (day 28) post confluency (FIG. 4a, FIG. 12). It was found that expression of each of these key transporters was higher in OPTECs compared to PTEC-TERT1 s subjected to the same conditions on chip. Importantly, it was observed that transporter expression was highest on day 14 for the OPTECs. A broader panel of transporters, including OCTs, OATs, endocytosis transporters, and glucose transporters was obtained for both OPTECs- and PTEC-TERT1 s-on-chip after 14 days of perfusion (FIG. 4b). Interestingly, significant upregulation was observed not only for OCT2 and OAT3, but also other key PT markers including AQP1/2, SGLT2, and glucose transporter 2, GLUT2. These findings indicate that compared to immortalized cell lines, the described OPTEC-based model may be more representative of in vivo PTs.

Cell polarity is essential for assessing toxicity and vectorial transport in microphysiologic analysis platforms. In kidney organoids (day 49, static), both organic cation and anion transporters are not properly polarized, as discussed above. Since OPTEC cells isolated from these organoids were used in the described 3D model, it was assessed whether perfusion on chip over a 14-day period enhanced the polarization of key transporters, OCT2 and OAT3, by IF imaging (FIG. 4c-f). As a benchmark, PTEC-TERT1 s perfused on chip over this same period were analyzed (FIG. 4g-j). Clear basolateral polarization of OCT2 and OAT3 were only observed in OPTEC tubules. In both cases, visual expression levels of these key markers appeared to also be higher in OPTEC tubules than those composed of PTEC-TERT1 s, in good agreement with the described Nanostring results.

Taken together, the transporter expression and polarization studies revealed that OPTECs exhibited higher levels of OCT2, OAT1, and OAT3, which were localized basolaterally. Hence, the described 3D OPTEC-on-chip model overcame the lack of proper polarization in statically cultured organoids and, importantly, more closely matched native proximal tubules.

### Nephrotoxic Drug Uptake and Inhibition

In this final set of experiments, the effects of known nephrotoxins, cisplatin and aristolochic acid, were assessed using the described 3D OPTEC-on-chip model.

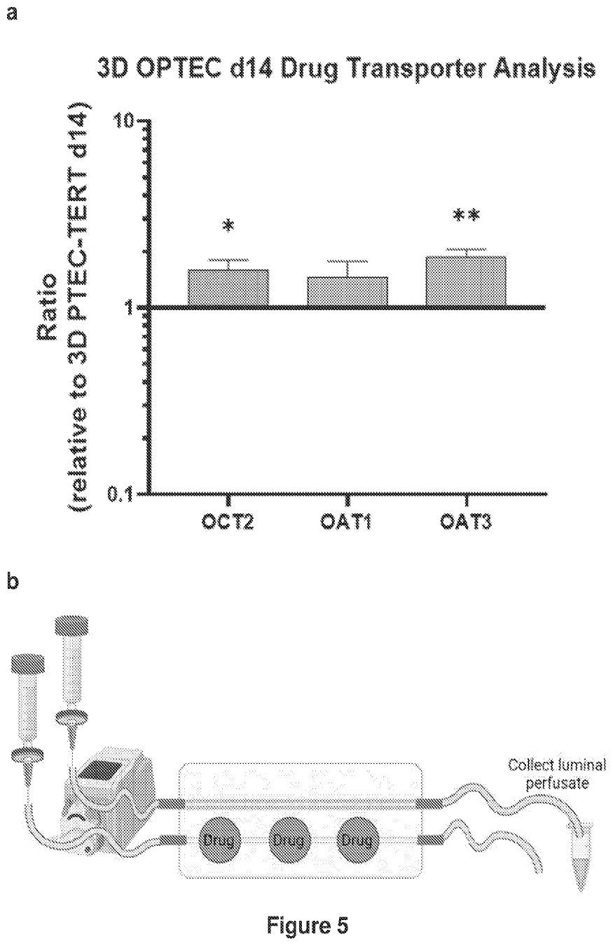

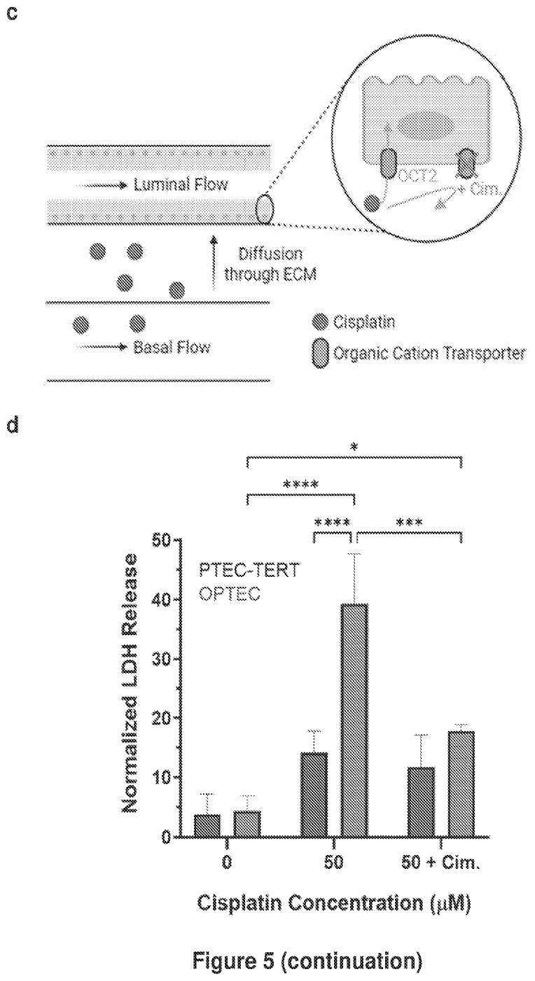

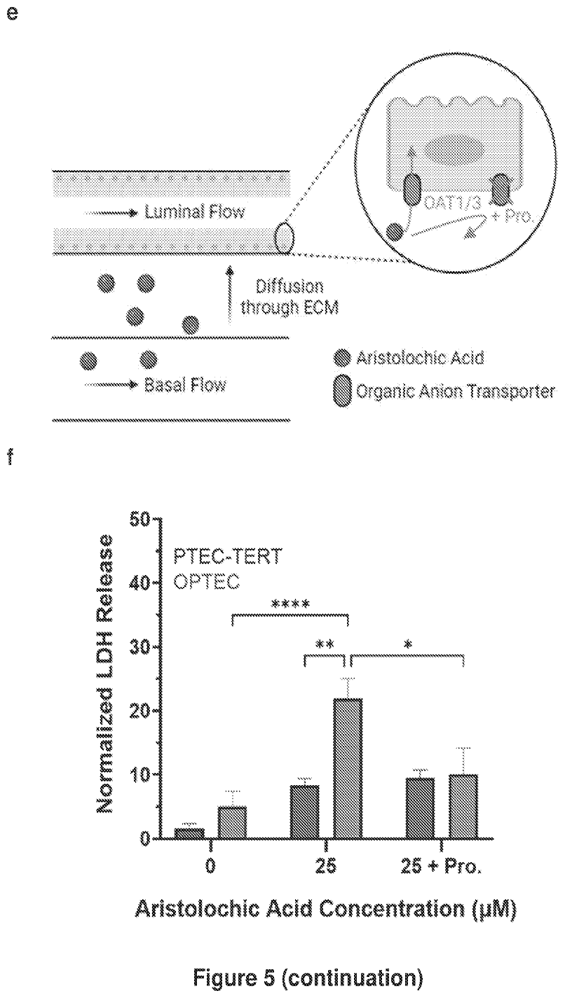

Nanostring data showed that OPTEC tubules (day 14) expressed significantly higher levels of OCT2 and OAT3 when compared to PTEC-TERT1 tubules (day 14), as shown in FIG. 5a.

To assess nephrotoxicity, luminal perfusate was collected and LDH analyses were performed. LDH is a cytosolic enzyme found in nearly all cell types, including PTECs, that is released upon cellular damage²⁹. To establish a baseline level of LDH, fresh media was perfused through the OPTEC and PTEC-TERT1 (control) chips for at least 4 h. Next, samples of luminal perfusate were collected from each chip for each LDH assay. Each drug was then added to the basolateral reservoir at appropriate concentrations and allowed to perfuse for 48 h (FIG. 5b). Luminal perfusate was collected at the 48-h timepoint followed by LDH analysis. Due to differences in their baseline LDH release among different tubules across the two cell types, a normalized LDH release (LDH release at 48 h relative to baseline release) was determined for each sample, i.e., OPTEC and PTEC-TERT1 tubules without drug, with drug, and with drug and corresponding inhibitors for OCT2 and OAT1/3 transporters, respectively.

Cisplatin is an anti-tumor drug that is known to accumulate in the PT inducing nephrotoxicity. Its uptake is mediated primarily by basolateral transporter OCT2 and causes PT injury through the generation of reactive oxygen species²⁷. When introduced through the basal channel on chip, cisplatin readily diffused through the gelatin-fibrin matrix, where PT cells can uptake the drug (FIG. 5c). No differences in normalized LDH release were observed between OPTECs and PTEC-TERT1 s in the absence of cisplatin. However, upon exposure to 50 μM cisplatin, OPTECs exhibited a significantly higher normalized LDH response compared to PTEC-TERT1 s. Upon introduction of cimetidine (1 mM), OCT2 inhibitor³⁰, the normalized LDH release was significantly decreased by more than a factor of 2 in the OPTEC tubules (FIG. 5d). By contrast, OCT2 inhibition had far less effect on the normalized LDH release observed for PTEC-TERT1 tubules exposed to the same cisplatin dose. These observations indicate that the enhanced OCT2 expression observed for perfused OPTEC tubules leads to a physiologically relevant increase in drug uptake via this transporter.

Aristolochic acid is a potent nephrotoxin that is transported basolaterally by OAT1 and OAT3²⁸. To determine the effects of aristolochic acid on the described 3D models, this drug was added to the basal reservoir and allowed to diffuse through the matrix for 48 h, where PT cells can uptake the drug (FIG. 5e). In the absence of aristolochic acid, no differences were observed in normalized LDH release between OPTEC and PTEC-TERT1 tubules. When 25 μM aristolochic acid was introduced, OPTEC tubules exhibited a significantly higher LDH release compared to PTEC-TERT1 tubules. Upon adding probenecid (2 mM), OAT1/3 inhibitor³¹, the normalized LDH release was again substantially reduced for the OPTEC tubules (FIG. 5f). By contrast, OAT1/3 inhibition elicited a negligible effect on the normalized LDH release observed for PTEC-TERT1 tubules exposed to the same aristolochic acid dose.

These observations further support the enhanced physiological relevance of the described 3D OPTEC-on-chip model for drug screening and toxicity testing.

### DISCUSSION

Advances in microphysiological systems have enabled the development of more physiologic proximal tubule and kidney organoid systems^{11, 13, 14, 18}. The inventors have previously shown the functional benefits of fluidic shear stress on 3D PT models^{13, 14} and kidney organoids¹⁸. By integrating biomanufacturing and kidney organoid development, demonstrated herein is a method for creating a perfusable, organoid-derived proximal tubule model on chip by isolating PTEC cells from kidney organoids and seeding them into 3D microenvironments that mimic native tubules.

Although a longer period is required to obtain OPTECs, due to the extended organoid differentiation, cell isolation, and expansion protocols, it was found that these cells exhibited the desired cuboidal morphology and rapidly assembled into a confluent epithelial monolayer when seeded in the form of 3D tubules. Upon perfusion, these OPTEC tubules exhibited proper polarization and expressed a broad range of proximal-tubule specific, functional markers. Importantly, they also exhibited a higher expression of basolateral drug transporters: OCT2, OAT1, and OAT3 compared to those based on immortalized PTECs. The observed decrease in transporter expression beyond day 14 may be addressed by incorporating peritubular fibroblasts into future embodiments of our model to provide additional stability³².

The described 3D OPTEC-on-chip model was used to investigate polarized drug uptake through organic cation and anion transporters in the absence and presence of transport inhibitors. The increased transporter expression and polarization observed in the described 3D OPTEC models after 14 days of perfusion directly translated to an observed increase in drug uptake and normalized LDH release by these tubules. Looking ahead, the described model can be extended in two important directions by, first, producing proximal tubules with more physiologically relevant diameters (approaching 60 μm in size) and, second, by seeding the empty (basolateral) channel with endothelial cells thereby creating a vascularized OPTEC-on-chip model.

In summary, described herein is the development and characterization of a 3D organoid-derived proximal tubule-on-chip model using cells isolated from kidney organoids. The described multiplexed, perfusable OPTEC tubule model establishes a more sensitive predictor of nephrotoxicity compared to traditional models based on immortalized PTECs. Additionally, the described work highlights the expected improvements that perfusion would confer on proximal tubular segments within kidney organoids subjected to physiologic luminal shear stresses. Finally, the use of PTECs obtained from hPSC-derived kidney organoids coupled with organ-on-a-chip methods opens new avenues for personalized drug screening and disease modeling.

### REFERENCES

1. Eaton D C, Pooler J P. Vander's renal physiology. Medicine. 2009; 2009:5.
2. Perazella M A. Renal vulnerability to drug toxicity. Clin. J. Am. Soc. Nephrol. 2009 doi: 10.2215/CJN.02050309.
3. Tiong H Y, et al. Drug-induced nephrotoxicity: Clinical impact and preclinical in vitro models. Mol. Pharm. 2014 doi: 10.1021/mp400720w.
4. Eric D, Gary M. Drug-induced renal injury. Contin. Med. Educ. 2011; 111:349.
5. Choudhury D, Ahmed Z. Drug-associated renal dysfunction and injury. Nat. Clin. Pract. Nephrol. 2006 doi: 10.1038/ncpneph0076.
6. Yu P, et al. Drug-induced nephrotoxicity assessment in 3D cellular models. Micromachines. 2022; 13:1-23.
7. Wilmer M J, et al. Kidney-on-a-chip technology for drug-induced nephrotoxicity screening. Trends Biotechnol. 2016 doi: 10.1016/j.tibtech.2015.11.001.
8. Lee J, Kim S. Kidney-on-a-chip: A new technology for predicting drug efficacy, interactions, and drug-induced nephrotoxicity. Curr. Drug Metab. 2018 doi: 10.2174/1389200219666180309101844.
9. van Duinen V, Trietsch S J, Joore J, Vulto P, Hankemeier T. Microfluidic 3D cell culture: From tools to tissue models. Curr. Opin. Biotechnol. 2015 doi: 10.1016/j.copbio.2015.05.002.
10. Seeker P F, Luks L, Schlichenmaier N, Dietrich D R. RPTEC/TERT1 cells form highly differentiated tubules when cultured in a 3D matrix. Altex. 2018 doi: 10.14573/altex.1710181.
11. Jang K J, et al. Human kidney proximal tubule-on-a-chip for drug transport and nephrotoxicity assessment. Integr. Biol. (U. K.) 2013 doi: 10.1039/c3ib40049b.
12. Vormann M K, et al. Implementation of a human renal proximal tubule on a chip for nephrotoxicity and drug interaction studies. J. Pharm. Sci. 2021 doi: 10.1016/j.xphs.2021.01.028.
13. Homan K A, et al. Bioprinting of 3D convoluted renal proximal tubules on perfusable chips. Sci. Rep. 2016 doi: 10.1038/srep34845.
14. Lin N Y C, et al. Renal reabsorption in 3D vascularized proximal tubule models. Proc. Natl. Acad. Sci. USA. 2019 doi: 10.1073/pnas.1815208116.
15. PromoCell. Human Primary Cells and Immortal Cell Lines: Differences and Advantages. Blog Lab, https://promocell.com/ (2019).
16. Jenkinson S E, et al. The limitations of renal epithelial cell line HK-2 as a model of drug transporter expression and function in the proximal tubule. Pflugers Arch. 2012 doi: 10.1007/s00424-012-1163-2.
17. Kim Y K, Nam S A, Yang C W. Applications of kidney organoids derived from human pluripotent stem cells. Korean J. Intern. Med. 2018 doi: 10.3904/kjim.2018.198.
18. Homan K A, et al. Flow-enhanced vascularization and maturation of kidney organoids in vitro. Nat. Methods. 2019 doi: 10.1038/s41592-019-0325-y.
19. Nieskens T T G, Wilmer M J. Kidney-on-a-chip technology for renal proximal tubule tissue reconstruction. Eur. J. Pharmacol. 2016 doi: 10.1016/j.ejphar.2016.07.018.
20. Wieser M, et al. hTERT alone immortalizes epithelial cells of renal proximal tubules without changing their functional characteristics. Am. J. Physiol. Ren. Physiol. 2008 doi: 10.1152/ajprenal.90405.2008.
21. Morizane R, Bonventre J V. Generation of nephron progenitor cells and kidney organoids from human pluripotent stem cells. Nat. Protoc. 2017 doi: 10.1038/nprot.2016.170.
22. Hale L, et al. 3D organoid-derived human glomeruli for personalised podocyte disease modelling and drug screening. Nat. Commun. 2018 doi: 10.1038/s41467-018-07594-z.
23. Takasato M, Er P X, Chiu H S, Little M H. Generation of kidney organoids from human pluripotent stem cells. Nat. Protoc. 2016 doi: 10.1038/nprot.2016.098.
24. Wu H, et al. Comparative analysis and refinement of human PSC-derived kidney organoid differentiation with single-cell transcriptomics. Cell Stem Cell. 2018 doi: 10.1016/j.stem.2018.10.010.
25. Morizane R, et al. Nephron organoids derived from human pluripotent stem cells model kidney development and injury. Nat Biotechnol. 2015 doi: 10.1038/nbt.3392.
26. Inman G J, et al. SB-431542 is a potent and specific inhibitor of transforming growth factor-β superfamily type I activin receptor-like kinase (ALK) receptors ALK4, ALK5, and ALK7. Mol. Pharmacol. 2002 doi: 10.1124/mol.62.1.65.
27. Arany I, Safirstein R L. Cisplatin nephrotoxicity. Semin. Nephrol. 2003 doi: 10.1016/S0270-9295(03)00089-5.
28. Xue X, et al. Critical role of organic anion transporters 1 and 3 in kidney accumulation and toxicity of aristolochic acid i. Mol. Pharm. 2011 doi: 10.1021/mp100418u.
29. Legrand C, et al. Lactate dehydrogenase (LDH) activity of the number of dead cells in the medium of cultured eukaryotic cells as marker. J. Biotechnol. 1992 doi: 10.1016/0168-1656(92)90158-6.
30. Ludwig T, RiethmOller C, Gekle M, Schwerdt G, Oberleithner H. Nephrotoxicity of platinum complexes is related to basolateral organic cation transport. Kidney Int. 2004 doi: 10.1111/j.1523-1755.2004.00720.x.
31. Chang S Y, et al. Human liver-kidney model elucidates the mechanisms of aristolochic acid nephrotoxicity. JCI Insight. 2017 doi: 10.1172/jci.insight.95978.
32. Kimura M, et al. Role of atrophic changes in proximal tubular cells in the peritubular deposition of type IV collagen in a rat renal ablation model. Nephrol. Dial. Transplant. 2005 doi: 10.1093/ndt/gfh872.

### Example 2

### INTRODUCTION

The kidney collecting duct is critical for urine modification and drainage and is a major site for disease and dysfunction. In vitro models of the collecting duct could expedite testing of disease phenotypes, drug interactions, and development. However, existing models are currently limited by the availability of appropriate human cell lines or lack of relevant biophysical cues such as flow. The recent development of protocols to derive ureteric bud (UB) and subsequently collecting duct-like cells directly from human pluripotent stem cells (hPSCs) presents an opportunity to advance in vitro models by enabling higher throughput in the context of relevant cell types and biophysical cues. Here, we develop perfusable models of the ureteric bud and collecting duct from hPSCs. To fabricate our model, we first demonstrate the scalability of existing UB derivation protocols by comparing growth, morphology, and gene expression of UB organoids cultured in conventional 96-well plates compared to microwells and transwell culture inserts. We find that both culture formats support similar UB growth rates, morphology, and marker expression. Measurement of intracellular Na+ concentration performed in UB tubules demonstrates Na+-K+-ATPase activity. Furthermore, UB organoids differentiated toward collecting duct-like organoids show increased collecting duct marker expression. Next, we demonstrate that organoids can be dissociated to single cells and seeded onto an extracellular matrix (ECM) and cultured to form confluent 2D monolayers. The presence of collagen I is necessary to prevent collapse of the monolayer over several days of culture. We then seeded UB cells into a 3D perfusable channel embedded within the optimized ECM composition. These UB cells on-chip form a confluent monolayer and maintain UB-like marker expression and morphology. Looking ahead, we plan to investigate flow effects on UB maturation and function with the ultimate goal of creating a human collecting duct model that can be used directly or embedded within biomanufactured kidney tissues.

### MATERIALS AND METHODS

IPSC Culture: BJFF human iPSCs (male, provided by Prof. Sanjay Jain at Washington University) were maintained in feeder-free culture on plates coated with 1% Matrigel in DMEM/F12 solution (list product) and cultured with mTeSR Plus medium (Stem Cell Technologies). For regular cell maintenance, cells were passaged at 60-80% confluency with ReLeSR according to manufacturer's directions and split at ratios of 1:8 to 1:20. Frozen stocks were maintained in a 1:1 cell embryonic freezing medium and mTeSR plus medium with 10 μM Y27632. Karyotype of cells were evaluated by WiCell to validate normal karyotype. Mycoplasma screening was performed at least every six months (Mycoalert, Lonza). Cells were passaged for no more than 15 passages past karyotyping.

Generation of UB/CD organoids: Human iPSCs were differentiated towards UB organoids following the protocol of Zeng et al. with minor adaptations (Zeng, Z., et al., 2021).

Briefly, iPSCs were washed with DPBS without Ca²⁺ and Mg²⁺ and dissociated from flask with Accumax solution for 5-10 min at 37° C. Cells were gently collected and equal volume mTeSR Plus medium with 1 μm Y27632 (mTeSR⁺Y) was added. Cells were spun down at 200 g for 2 min and resuspended in mTeSR⁺Y. Then, 60,000 cells in 1 mL of mTeSR⁺Y were seeded per well into a 1% Matrigel pre-coated 12-well plate or 1.2 million cells in 22 mL of mTeSR+Y into a T75 flask. Medium was replaced with pre-warmed ME-medium (Table 3) day after seeding. At day 3 the ME-media was replaced by 1 mL of pre-warmed UB-I medium (Table 3), medium was refreshed the following day. At day 5, UB-I medium was removed and replaced with 2 mL of UB-II medium (Table 3). The UB-II medium was refreshed the next day. On day 7, differentiated cells were washed with 1 mL/well of DPBS without Ca²⁺ and Mg²⁺ and then dissociated with 0.5 mL per well of Accumax solution for 3-10 min at 37° C. Cold FACS buffer (DPBS with 1% (vol/vol) P/S and 2% (vol/vol) FBS) was added in equal volume to the collected cells and cells were spun down at 300 g for 5 min at 4° C. For cell sorting, the cells were resuspended in 2 mL of cold FACS buffer and 2 μL per initial 12 well plate of anti-CD117 PE-antibody was added. Samples and antibody were kept on ice for 30 min. The tube was gently tapped every 10 min to ensure mixing. After 30 min, samples were washed with cold FACS medium and resuspended in 2 ml per plate cold FACS medium supplemented with 1:4000 DAPI. Cells were strained over 40-μm cell strainer (Greiner bio-one, Cat. No. 542040) and transferred into a FACS tube. CD117⁺DAPI⁻ cells were sorted with either a Beckman Coulter MoFlo Astrios EQ or a BD FACS Aria in the Bauer Core and collected in FACS buffer. Cells were spun down at 300 g for 5 min and resuspended in UBCM media with 1 um Y27632.

After sorting or dissociation, cells were aggregated either in a U-bottom 96 ultra-low adherent well plate (96ULA) or in an AggreWell™ 800 24-well Plate (Aggrewell). In the 96ULA format, 4,000-10,000 cells were seeded per well in UBCM media with 1 um Y27632. Cells were allowed to spontaneously aggregate for 1 day, following which the media was removed and aggregates were embedded in 15 μL 1:1 Geltrex:UBCM gels. After 30 min gelation at 37° C. UBCM media was added and half medium changes were performed every 2-3 days during subsequent differentiation. Cells were cultured at 5% CO2 at 37° C. In the Aggrewell format, 4,000-10,000 cells in UBCM media with 1 um Y27632 were seeded per microwell according to manufacturer's instructions. Aggregates were formed by spinning the plate at 100 g for 3 min. The following day aggregates were utilized for experiments or embedded for further differentiation in a transwell system. To embed, 1 mL of 1:1 Geltrex:UBCM was added to a 6 well transwell insert and incubated for at least 30 min at 37 deg C. Aggregates were collected from the Aggrewell and resuspended in 0.5 mL 1:1 Geltrex:UBCM and placed on top of the solidified Geltrex:UBCM within the insert, and incubated for at least 30 min at 37 deg C. After gelation, UBCM was added to the outer compartment and media changes were performed every 2-3 days during subsequent differentiation. Finally, CD organoids were generated by removing hUBCM medium from 21-35 day old embedded UB organoids and adding CD medium and culturing for an additional 7 days. Medium was refreshed every 2-3 days for at least 5 days. Cells were cultured at 5% 002 at 37° C.

TABLE 3
Differentiation medium compositions.

| | | Final | |
|---|---|---|---|
| Additives | Reagent Name | Concentration | |
| Base medium (all | GlutaMAX (100x) | 1x | |
| added to | MEM NEAA (100x) | 1x | |
| DMEM/F12, | Pen Strep (100x) | 1x | |
| HEPES | B-27 Supplement minus vitamin | 1x | |
| | A (50x) | | |
| | ITS Liquid Media Supplement | 1x | |
| | (100x) | | |
| ME Additives | LDN-193189 | 10 | nM |
| (add to base | CHIR99021 | 4.5 | μM |
| medium) | | | |
| UB-I Additives | FGF2 | 200 | ng/mL |
| (add to base | TTNPB | 0.1 | μM |
| medium) | LDN-193189 | 30 | nM |
| | A83-01 | 0.2 | μM |
| UB-II Additives | FGF2 | 200 | ng/mL |
| (add to base | TTNPB | 0.1 | μM |
| medium) | LDN-193189 | 30 | nM |
| hUBCM | LDN-193189 | 200 | nM |
| (add to base | TTNPB | 0.1 | uM |
| medium) | CHIR990021 | 1 | μM |
| | JAK inhibitor I | 100 | nM |
| | GDNF | 100 | ng/mL |
| | A83-01 | 0.2 | μM |
| | Rspo1 | 100 | ng/mL |
| | FGF7 | 50 | ng/mL |
| | SB202190 | 5 | μM |
| | EGF | 50 | ng/mL |
| CD | KSR | 3% | v/v |
| (add to base | Aldosterone | 100 | nM |

| | | |
|---|---|---|
| medium) | Vasopressin | 1 I.U./mL |

ECM Preparation: Gelbrin was prepared as a mixture of gelatin and fibrinogen. To prepare the ECM components, a 15% (w/v) gelatin solution (Type A, 300 bloom from porcine skin; Sigma) was formed by adding gelatin powder to a warm solution (70° C.) of DPBS without Ca²⁺ and Mg²⁺. The gelatin was stirred for 12 h at 70° C., and the pH is then adjusted to 7.5 with 1 N NaOH. The solution was sterile-filtered and stored at 4° C. in aliquots for later usage. A fibrinogen solution (50 mg/ml) was made from lyophilized bovine blood plasma protein (Millipore) dissolved at 37° C. in sterile DPBS without Ca²⁺ and Mg²⁺. The solution is held at 37° C. without agitation for at least 45 min to allow complete dissolution. The transglutaminase solution (60 mg/ml) was made from lyophilized powder (Moo Gloo, TI) dissolved in DPBS without Ca²⁺ and Mg²⁺ and gently mixed for 20 s. The solution is then held at 37° C. for 20 min and sterile-filtered before use. A CaCI₂ stock solution (250 mM) was prepared from CaCl₂ pellets dissolved in sterile water. For the preparation of stock solutions of thrombin, lyophilized thrombin (Sigma Aldrich) was reconstituted at 500 U/ml in sterile water and stored at −20° C. Thrombin aliquots were thawed immediately before use. Before the gelbrin was cast, several components are mixed in advance at appropriate concentrations, including 10 mg/ml fibrinogen, 2% (wt/vol) gelatin, 2.5 mM CaCl₂, and 0.2% (vol/vol) transglutaminase. This solution was then equilibrated at 37° C. for 15-20 min before use to improve the optical clarity of the gelbrin. Next, the solution was rapidly mixed with stock thrombin solution at a ratio of 250:1, resulting in a final thrombin concentration of 2 U/ml. Within 2 min at 37° C., soluble fibrinogen cures to a fibrin gel. For this reason, the ECM solution must be cast onto the culture plate immediately after being mixed with thrombin and incubated for at least 30 min before use.

Fibrin gels were formed by first preparing a solution of 10 mg/ml fibrinogen and 2.5 mM CaCl₂ in DPBS without Ca²⁺ and Mg²⁺ from the 50 mg/mL fibrinogen and 250 mM CaCl₂ stock solution. Next, the solution was rapidly mixed with stock thrombin solution at a ratio of 250:1, resulting in a final thrombin concentration of 2 U/ml. Within 2 min at 37° C., soluble fibrinogen cures to a fibrin gel.

Type I bovine collagen (TeloCol-10, Advanced Biomatrix, Cat #5226) was prepared at 1 mg/mL final concentration. For gel, 1 part of chilled collagen solution was mixed with 8 parts sterile water 1 part 10×PBS. The pH of the mixture was adjusted to 7.2-7.6 using sterile 1 N NaOH and pH was monitored using pH paper. To prevent gelation, the solution was maintained on ice at 2-10° C. until use. Finally, the solution was dispensed into culture plates and incubated at 37° C. for approximately 30 to 60 minutes to form gel.

Geltrex LDEV-Free Reduced Growth Factor Basement Membrane Matrix (ThermoFisher, Cat #A1413202) was mixed with DPBS or cell medium and cells to prepare gels. For organoid embedding in transwells or 96-wells, geltrex was mixed 1:1 with hUBCM or UBCM with organoids. For 2D and 3D screening assays 86% (vol/vol) geltrex was mixed with one of the following: 14% (vol/vol) DPBS, 10% (vol/vol) of 10.0 mg/mL collagen I stock solution and 4% (vol/vol) of transglutaminase stock solution, 10% of and 4% (vol/vol), or 10% DPBS.

Methacrylated hyaluronic acid (MeHA) containing ECMs were prepared as follows. Methacyrlated HA (Nanosoft Polymers, 50 kDa) was dissolved at 10% (wt/vol) in DBPS overnight at 4 deg C. to form stock solution. A stock solution of dithiothreitol (DTT) was dissolved at 10 mg/mL in DBPS and sterile filtered. A lithium phenyl-2,4,6-trimethylbenzoylphosphinate (LAP) photoinitiatior stock was formed by dissolving LAP in deionized sterile water overnight at 4 deg C. to form a 5% wt/vol solution. Collagen I (TeloCol-10, 10.0 mg/mL) was added directly. Finally, geltrex stock (considered as 100% (wt/vol) from the manufacturer was added directly. Stock solutions were mixed with DPBS to form ECM with concentrations of 0.5-1.5 mg/mL collagen, 0.75%-1.75% (wt/vol) MeHA, DTT at 1 mg/mL for 1% (w/v) of MeHA, 0.05% wt/vol LAP, and 0%-50% vol/vol geltrex. These ECMs were cast and equilibrated overnight at 37 deg C. in a humidified incubator. The next day, ECMs were photocrosslinked via UV light for 120 s on top and 120 s on bottom. ECM could be further coated with 1% (w/v) geltrex or Matrigel in DMEM/F12 and/or polylysine at 0.1 mg/mL in DBPS.

2D ECM Cell Adhesion Assay: ECM substrates for 2D substrates were prepared in the bottom of a 48-well glass bottom plate. First, glass was coated with 45 μL of Poly-D-Lysine for 1-2 min and removed by pippetting. Wells were washed with 0.5 mL of sterile MilliQ water and aspirated dry and then left at room temperature to further dry overnight. To the glass region of each well, 45 μL of ECM solutions were cast and allowed to crosslink for ˜1 hr. As a control, glass wells were coated with 45 μL of 1% Matrigel solution in DMEM/F12. To maintain humidity in plates, sterile MilliQ water was added to the outside wells and in between wells. Collagen, fibrin, and gelbrin gels were prepared up to 2 days in advance and stored at 4 deg C. in sealed, sterile containers. Then, UB organoids within gel were harvested from transwells at day 21. To dissociate the embedded organoids, organoids were pipetted ˜10× to break up matrigel and resuspended with 10 mL of DPBS without Ca²⁺ and Mg²⁺. The mixture was then centrifuged at 300 g×5 min, yielding a distinguishable cell pellet and geltrex layer. The excess DPBS without Ca²⁺ and Mg²⁺ and geltrex layer was removed by aspiration, and the cell layer was then incubated with 1 mL of 0.25% trypsin for every 1 transwell from which organoids were collected for 30 min at 37° C. Gentle manual pipetting was done every ˜10 min during and at the end of the incubation to dissociate cells. Samples were washed with PBS^{+/+}, strained over 40-μm cell filter and counted. Cells were seeded on 2D substrates at a density of 385,000 cells/cm² in a 50 μL UBCM with 10 μm Y27632 droplet and left to adhere for ˜1 hr. Subsequently, 300 μL of UBCM with 10 μm Y27632 was added to each well. The following day media was replaced with 300 μL of UBCM per well. Samples were fixed on the following day, 2 days post seeding. Fixed samples were permeabalized with 0.1% Triton X and stained with DAPI and 488 phalloidin. Finally, samples were imaged and confluency was analyzed using ImageJ analysis software.

3D ECM UB Growth and Morphology Assay: After aggregation (1 day post FACS), organoids were embedded in 3D ECM. First, a 200 μL layer of ECM was placed into a 12 mm transwell culture insert and incubated at 37° C. for ˜1 hr. Then, 200 μL ECM was mixed with approximately 10-25 organoids μL in 20 μL of medium and was cast on top of the first layer of ECM. The ECM and organoids were incubated for an additional ˜1 hr. Finally, 1.5 mL of UBCM was added to the bottom of the well and changed every 2-3 days. Phase images were taken at day 1 (day of embedding, 1 day post FACS), day 7, day 14, and day 21. At day 21, samples were fixed.

Immunostaining: Organoids were stained according to protocol previously reported by Homan et al. Prior to immunostaining, each sample was washed with DPBS and then fixed for 1 hr with 10% (vol/vol) buffered formalin. The fixative was removed by 2 washes in PBS for ˜2 hrs, and samples were then blocked overnight with 1% (vol/vol) donkey serum in PBS++ with 0.125% (vol/vol) Triton X-100. Primary antibodies to the protein or biomarker of interest were incubated with the constructs for 2 d at 4° C. at the dilutions listed in Table X in a solution of 0.5% (wt/vol) BSA and 0.125% (vol/vol) Triton X-100. Removal of unbound primary antibodies was accomplished via a wash step against a solution of PBS or 0.5% (wt/vol) BSA and 0.125% (vol/vol) Triton X-100 in PBS for 1 d. Secondary antibodies were incubated with the constructs for several hours at dilutions listed in Table 2 in a solution of 0.5% (wt/vol) BSA and 0.125% (vol/vol) Triton X-100 in PBS. Samples were counterstained with DAPI and then washed for at least several hours in PBS before imaging. Comprehensive lists of resources and reagents, as well as antibodies can be found in Tables 4-6 below.

TABLE 4
Resources and Reagents

| | | |
|---|---|---|
| REAGENT or RESOURCE | SOURCE | IDENTIFIER |

Chemicals, Peptides, and Recombinant proteins

| | | |
|---|---|---|
| DMEM/F12, HEPES | FisherScientific | Cat#11-330-032 |
| mTeSR Plus | STEMCELL Technologies | Cat#100-0276 |
| Accumax | Innovative Cell | Cat#AM-105 |
| | Technologies | |
| Fetal bovine serum | | |
| Matrigel | | Cat#356230 |
| Geltrex | Gibco | Cat#A14132-02 |
| TeloCol ®-10 Type I Collagen | Advanced BioMatrix | Cat#5226 |
| Solution, 10 mg/ml (Bovine) | | |
| 0.25% Trypisin-EDTA | Gibco | Cat#25200-056 |
| Fibrinogen, Bovine | EMD Millipore | 341573 |
| Transglutaminase | | |
| Thrombin | | |
| CaCl₂ | Alfa Aesar | 89866 |
| Gelatin | Sigma-Aldrich | G1890 |
| DPBS | Corning | 21-030-CV |
| DPBS−/− (without Ca²⁺ or Mg²⁺) | Corning | 21-031-CV |
| GDNF | Peprotech | 450-10 |
| FGF2 | | |
| FGF7 | Peprotech | 100-19 |
| EGF | Peprotech | AF-100-15 |
| CHIR 99021 | Tocris Bioscience | 4423/10 |
| LDN | | |
| A83-01 | Reprocell | 04-0014 |
| TTNPB | Tocris Bioscience | 0761 |
| Y27632 | Tocris Bioscience | 1254 |
| BSA | Roche | 10735078001 |
| R-Spondin-1 | Peprotech | 120-38 |
| B27 minus Vitamin A | Fisher | Cat#12587010 |
| [Arg8]-Vasopressin | Sigma-Aldrich | Cat#V0377- |
| | | 100IU |
| ITS supplement | Gibco | 41400-045 |
| MEM NEAA | Gibco | 11140-050 |
| Glutamax | Gibco | 35050-061 |
| PenStrep | Gibco | 15140-122 |
| ReLeSR | STEMCELL Tehcnologies | 100-0483 |
| Embryonic Stem Cell Freezing | Millipore | ES-002-F |
| Med. | | |
| DTT | Roche | 51033324 |
| Methacrylated Hyaluronic Acid | Nanosoft Polymers | 13204-50 K |
| Poly-D-Lysine | Sigma-Aldrich | Cat#P6407 |
| LAP | Sigma-Aldrich | 900889-1G |
| MycoAlert Mycoplasma Kit | Lonza | LT07-118 |
| RNeasy Plus Mini Kit | Qiagen | 74134 |
| Anti-Adherence Rinsing Solution | Stemcell Technologies | 07010 |
| Collegenase Type IV (1 mg/mL) | Stemcell Technologies | 9001-12-1 |

Other

| | | |
|---|---|---|
| Aggrewell ™ 800 | STEMCELL Technologies | Cat#34815 |
| Transwell (6-well) Polyester 0.4 | Corning | Cat#3450 |
| μm pore size | | |
| Transwell (12-well) PET 0.4 μm | Corning | Cat#353180 |
| pore size | | |
| ULA 96-well plate | Costar | 7007 |
| 316 Stainless Steel Tubing, 0.05″ | McMaster-Carr | Cat#89935K625 |
| OD, 0.006″ Wall Thickness | | |
| 316 Stainless Steel Tubing, | McMaster-Carr | Cat#89875K361 |
| 0.034″ OD, 0.004″ Wall | | |
| Thickness | | |
| 48-well glass bottom dishes, No. | MatTek Life Sciences | Cat#P48G-1.5-6- |
| 1.5 Coverslip, 6 mm diameter, | | F |
| uncoated | | |
| Pump | VWR | MFLX78001-40 |
| Silicone tubing | Cole Parmer | 06411-60 |
| PharMed BPT 0.25 ID tubing | Ismatec | 95723-12 |
| Glass slides | VWR | 16004-422 |
| Chip Lid | Celltreat | 229164 |

TABLE 5
Antibodies

| | | | | |
|---|---|---|---|---|
| | Host | | | Dilu- |
| Target | Species | Manufacturer | Product # | tion |

Primaries

| | | | | |
|---|---|---|---|---|
| KRT8 | Rat | DSHB | TROMA-1 | 1:50 |
| E-Cadherin | Mouse | Abcam | ab1416 | 1:100 |
| ETV5 | Rabbit | Abcam | ab102010 | 1:400 |
| SOX9 | Rabbit | Abcam | ab185230 | 1:300 |
| PAX2 | Rabbit | ThermoFisher | 71-6000 | 1:200 |
| | | Scientific | | |
| GATA3 | Rabbit | Cell Signaling | 5852T | 1:100 |
| ATVP6V1B1 (IC) | Rabbit | Abcam | ab192612 | 1:200 |
| AQP2 | Rabbit | Abcam | ab62628 | 1:200 |
| ENaC | Rabbit | Invitrogen | PA1-920A | 1:200 |
| Na + K + ATPase | Rabbit | Abcam | ab76020 | 1:200 |
| (PC) | | | | |
| RET | Goat | R&D technologies | AF1486 | 1:100 |
| CD117 | Mouse | BioLegend | 313204 | 1:300 |
| Acetylated Tubulin | Rabbit | Cell Signaling | 5335 | |
| | | Technologies | | |

Secondaries

| | | | | |
|---|---|---|---|---|
| 488 anti Rat | Donkey | ThermoFisher | A21208 | 1:400 |
| | | Scientific | | |
| 546 anti Mouse | Donkey | ThermoFisher | A10036 | 1:400 |
| | | Scientific | | |
| 647 anti Rabbit | Donkey | ThermoFisher | A32795 | 1:300 |
| | | Scientific | | |
| 647 anti Rat | Donkey | Abcam | AB150155 | 1:200 |
| 555+ anti Rabbit | Donkey | ThermoFisher | A32794 | 1:300 |
| | | Scientific | | |
| 488 anti Mouse | Donkey | ThermoFisher | A21202 | 1:300 |
| | | Scientific | | |
| 546 anti Goat | Donkey | ThermoFisher | | 1:300 |
| | | Scientific | | |

TABLE 6
Cell stains

| Reagent | Manufacturer | Product # | Dilution |
|---|---|---|---|
| DAPI (4′,6-diamidino-2- | | | 1:1000 |
| phenylindole) | | | |
| ActinGreen 488 ReadyProbes | ThermoFisher | Cat#R37110 | 1:100 |

### 3D Model Fabrication:

Custom 3D perfusable chips were fabricated as follows (FIG. 17A). Metal bases were machined from 0.12″ thick stainless steel sheets (McMaster-Carr, 8983K93). Gaskets and reservoir lids were acquired from CellTREAT devices (Cat #229164). Custom chips were printed using a FormLabs 3B+ printer in Biomed Clear ink. Chips were washed in isopropyl alcohol for 15 minutes and cured for 1 hour at 60 deg C. in the Formlabs Form Cure. The chips included 3D printed adapters which could be linked to tubing for perfusion of individual channels. A 6 mm thick cast acrylic reinforcement lid was added which created an external reservoir on each chip that could hold water for additional humidification, contain any spills, and be labeled.

The inner chip reservoir housed within the chip was filled with ˜500 μL of a hydrogel extracellular matrix (ECM) as described above and a pin (McMaster, 1 mm OD) was added to mold the channel. The ECM solution placed within the chip reservoir such that the tubule pin was fully immersed within the ECM (FIG. 17B). The chip lid was added and the assembled chip was then covered by a plastic dish to prevent evaporation and contamination and held at 37° C. for 1 h to allow for collagen or geltrex crosslinking. Afterwards, 200 μL of DPBS with 1× antibiotic-antimycotic (Gibco, 15240062) was added to each chip reservoir to hydrate the ECM and the matrix was equilibrated at humidified 37 deg C. overnight. The next day, the DPBS was removed and if the ECM contained MeHA, the ECM was photocrosslinked in a UV-light chamber for 120 s on top and 120 s on bottom. The tubule pin was removed through the outlet conduit, leaving a hollow channel embedded within the ECM-on-chip.

The fabricated 3D chips were then connected to external cell culture media reservoirs to enable media flow and cell seeding within the channel. Unless prepackaged sterilely, each component described below was autoclaved before use. A 10-ml syringe barrel and adapter assembly (EFD Nordson, East Providence, RI) with a 0.20-μm syringe filter (Corning Inc.) served as a media reservoir. Microbore silicone tubing (0.50-mm inner diameter) (Cole-Parmer) connected the reservoir to the pump inlet, the pump outlet to the chip inlet, and the chip outlet back to the reservoir (FIG. 16A). The circuit was primed and perfused with UBCM+RI medium with a peristaltic pump (Ismatec) using two-stop PharMed BPT tubing (0.25 mm ID) (Cole-Parmer). Polypropylene pinch clamps were added to the silicone tubing to prevent uncontrolled flow when the circuit was disconnected from the pump such as during cell culture medium changes. The 3D model was then placed in the incubator and perfused with medium for 24 h before cell seeding to fully saturate the ECM with media. The medium reservoir was equilibrated with atmospheric incubator conditions by means of a sterile filter on top of the reservoir.

To seed the channel with cells, UB cells day 7-21 were dissociated from transwells as described above and concentrated in UBCM+RI to a density of ˜100×10⁶ cells/mL. Alternatively, cells at day 0 could be seeded directly. Then, 20 μL of cell suspension was then injected into the empty channel through the outlet and then construct was incubated at 37° C. and 5% CO₂ for 1-2 hrs without flow. During this incubation, the chip was rotated without medium in the chip reservoir every ˜20 minutes to thoroughly coat channels. After this incubation, 1 mL of hUBCM+RI was added to each chip reservoir and chips were incubated overnight. The next day, nonadherent cells were flushed out with hUBCM+RI and the chip reservoir medium was refreshed.

After 2-7 more days (when channels appeared confluent) the chips were connected to perfusion reservoir and constructs were continuously perfused using a unidirectional peristaltic flow rate of 5 μL/min. After 1 day of perfusion at 5 μL/min, flow rate was either maintained at 5 uL/min (low flow) or increased to 60 μL/min (high flow) equating to a physiologic FSS for the CCD of 0.1 dyn/cm² for further study. After 1 more day, some chips were changed to CD medium. Chip reservoir and perfusion reservoir mediums were changed every 2-3 days and cultured from 2 days to 2 weeks. All media on chip included amphotericin B at 0.25 ug/mL.

### Rheology:

A controlled stress rheometer (DHR-3, TA Instruments, New Castle, DE, USA) with a 25-mm-diameter plate (disposable aluminum, roughened) plate geometry with a gap of 1000 μm was used to measure the rheological properties of the ECM. For time sweeps, a premixed ECM solution was rapidly placed onto the Peltier plate at 4° C. with the temperature subsequently ramped and held at 37° C. in a humidified chamber. The shear storage (G′) and loss (G″) moduli were measured at a frequency of 0.1 Hz and an oscillatory strain (y) of 0.1%. Rheological testing consisted of frequency sweeps ranging from 100 to 0.1 Hz at 0.1% amplitude also in a humidified 37° C. chamber. Shear modulus was reported as the average storage modulus for 3 tests per matrix composition at an oscillation frequency of 0.5 Hz.

### RNA Isolation and Nanostring Analysis:

For each sample, 6-10 organoids were collected. Organoids were washed with DBPS and then treated with a 1:1 solution of collagenase IV and DPBS for 30 min. After treatment organoids were washed with DPBS without Ca2+ and Mg2+ and then lysed. Adherent 2D iPSCs were first treated with accutase and pelleted at 300 g×5 min. Excess medium was removed and then cells were lysed. RNA was isolated using RNeasy Plus Mini Kit (Qiagen) according to manufacturer's instructions. RNA concentration was assessed using both the Nanodrop 1000 spectrophotometer (NanoDrop Products, Thermo Scientific, Wilmington, DE) and Qubit (Life Technologies). A custom panel was designed, and assay was carried out using the NanoString nCounter Elements™ protocol per the manufacturer's instructions. All procedures regarding sample preparation, hybridization, detection and scanning were performed as recommended by NanoString Technologies (NanoString, Seattle, WA). The custom probes (A and B) were designed by IDT (IDT Technologies, Coralville, USA) and contained 35-50 bp each (Table 7). Probes were diluted to a final concentration of 0.6 nM (probe A) and 3.0 nM (probe B) to create the 30× working probe pools. The total amount of 100 ng RNA was used. RNA was hybridized with probe pools, hybridization buffer and TagSet reagents in a total volume of 30 μl and incubated at 67° C. for 20 h. Samples were then loaded to the automated nCounter Sample Prep Station (NanoString Technologies, Seattle, WA), which performed the purification steps and cartridge preparation. Finally, the cartridges containing immobilized and aligned reporter complexes were transferred to a nCounter Digital Analyzer (NanoString Technologies), and expression data were subsequently generated using the high-resolution setting, which takes 600 images per sample. Analysis was performed in NSolver.

TABLE 7
Nanostring probe sequences.

| | | Mark- | | | | | |
|---|---|---|---|---|---|---|---|
| | | er | Acc- | SEQ | | SEQ | |
| | Mark- | ref- | ession | ID | | ID | |
| Gene | er | erence | # | NO: | Probe A sequence | NO: | Probe B sequence |
| AA | LH | | NM_ | 1 | TATGCTATGAGGATCCCCACAATCAGAA | 2 | CGAAAGCCATGACCTCCGATCACTC |
| DA | | | 001086.2 | | GGTACAGCGATTTTCTTCCCATCAGGTC | | TCATTCTCCATGGCTCCTCAACGTT |
| C | | | | | TCGATCTCGTACAAAACGACTATGACCA | | ATCTGGGAGAGGCGTATAAATATAA |
| | | | | | T | | |
| AB | PT | | NM_ | 3 | TTCTGGCCACCAGAGAGCTGAGTTCCTT | 4 | CGAAAGCCATGACCTCCGATCACTC |
| CB | | | 000927.3 | | TGTCTCCTACTTTAGTGCTATACTTACGG | | GCAAAATATGAGGCTGTCTAACAAG |
| 1 | | | | | TTTTGACATTCCGGAAATTTGGATCGAA | | GGCACGAGCTATGGCAATGCGTTG |
| | | | | | | | T |
| AB | PT | | NM_ | 5 | GAAGTCTGGCTGCCAAAACTCCTCCGTG | 6 | CGAAAGCCATGACCTCCGATCACTC |
| CC | | | 004996.3 | | GGAAGCACCAGGAAACCACTTGCAGAA | | CAGTAGGCTGAATTTGGTCACTAGG |
| 1 | | | | | GATCAAAAAACGATCCCTGTCCATCAAT | | ATCTTCTATACAACCAATTCCTCCA |
| | | | | | AC | | |
| AB | PT | | NM_ | 7 | CAGCACAGTTACCCTGTAAGGCACTTTA | 8 | CGAAAGCCATGACCTCCGATCACTC |
| CC | | | 003786.3 | | CAGGTGCAAAATCTTAAGGTGCCTAGCC | | CTTAGGCGTGTCATTCACCACTTGG |
| 3 | | | | | CGCGCAACTCGTCCTATTAAAAGTCGGA | | GGATCATTTCCTCATCTAAAGCATT |
| | | | | | A | | |
| AB | PT | | NM_ | 9 | TGCACTGAGAGGATCGTCCAGGAGATA | 10 | CGAAAGCCATGACCTCCGATCACTC |
| CC | | | 00110551 | | GATGTCAGCATCTTGATACACTGCCTGC | | ATTTGACAAATACACAGTTCGAACA |
| 4 | | | 5.2 | | ATTCTCATGGAAATGCAATGGATTCATTC | | AGTGTCTGCTAACTTCCGCATCTAC |
| | | | | | C | | |
| AC | IJ | | NM_ | 11 | AGCAGGAAAGTGTTTTAGAAGCATTTGC | 12 | CGAAAGCCATGACCTCCGATCACTC |
| TA | | | 001613.3 | | GGTGGACAATGGAAGGCCCGGCCTACG | | AAGGCATAATTCCACAGGACATTCA |
| 2 | | | | | GTTACCGTCTTTATAAGTGAACAAAACC | | CAGTTGTGTGCTAGAGACAGAGAG |
| | | | | | GG | | G |
| AC | HK | | NM_ | 13 | ATCGGCAAAGGCGAGGCTCTGTGCTCG | 14 | CGAAAGCCATGACCTCCGATCACTC |
| TB | | | 001101.3 | | CGGGGCGGACGCGGTCTCGCATTTGGA | | CGATATCATCATCCATGGTGAGCTG |
| | | | | | ATGATGTGTACTGGGAATAAGACGACG | | GCGGCGGGTGTGGACGGGCGGCG |
| | | | | | | | G |
| AG | SC | | NM_ | 15 | TTGGAAACTGGACAGAACAATCTGGAAC | 16 | CGAAAGCCATGACCTCCGATCACTC |
| TR | | | 000685.3 | | TCTCATCTCCTGTTGCTCCTCTCAGAGTT | | GTAGCACAGTTGCTAATAGCTGAAA |
| 1A | | | | | GTCAAGACGTTGGCGTGATGGGATGAA | | ACCGGCACGAAAACTTTACTGCCCT |
| | | | | | C | | |
| AL | SC | | NM_ | 17 | GGCGCGGGGCAGGGGTCCGAAGGCCT | 18 | CGAAAGCCATGACCTCCGATCACTC |
| X1 | | | 006982.3 | | GCACGCATTTGCCCTTGGTGAAAAGTCT | | TGCTGTCCTGACAGGGCGAGGTCC |
| | | | | | CTTGTTATTTTGTGCTCGC | | TCTCCAAGCGCACGTGATGCTC |
| AQ | PT | | NM_ | 19 | AGAAGGAGGGCCGTGCCCAAAGGCACA | 20 | CGAAAGCCATGACCTCCGATCACTC |
| P1 | | | 198098.1 | | AAGCAATTACGGTAGAATCCCAGCACAA | | TCTTCCCCCTCCAAGCACCATTGGG |
| | | | | | GAATCCCTGCTAGCTGAAGGAGGGTCAA | | AGCAAGGTGCATGTTAGGAAA |
| | | | | | AC | | |
| AQ | CD- | | NM_ | 21 | AAAAAGGTCTGAGTTCAAGTGTTGGCTC | 22 | CGAAAGCCATGACCTCCGATCACTC |
| P2 | DT | | 000486.5 | | CACCATATACTACCTGTGAGACCAGATA | | TCCCAGGAGCCTAGGATTTGTCTCA |
| | | | | | AGGTTGTTATTGTGGAGGATGTTACTAC | | GTGGAAAAATCATGGGATTTTGTGT |
| | | | | | A | | |
| AQ | CD- | | NM_ | 23 | TTTCCTCTCCTTATGCACAGATGGACAG | 24 | CGAAAGCCATGACCTCCGATCACTC |
| P3 | DT | | 004925.3 | | GCTGCCTTCCCTTTCGTTGGGACGCTTG | | CTCCTCCATGTGAAGCCCCTGAAAC |
| | | | | | AAGCGCAAGTAGAAAAC | | ATACACACCCTGGAAC |
| AQ | CD- | | NM_ | 25 | AATCAGGTTTAATAGTGGTCCATTATCTC | 26 | CGAAAGCCATGACCTCCGATCACTC |
| P4 | DT | | 004028.3 | | ACTGCCACAGACACACAACTGCCAGCAG | | TGACGACTCTCTGGGAATCTCACAC |
| | | | | | ACCTGCAATATCAAAGTTATAAGCGCGT | | ATCACTCTTTCCTAGCACCGAAGAG |
| AS | SC | | NM_ | 27 | GGTCTACAGTTGATTGTAGCATTGTGGT | 28 | CGAAAGCCATGACCTCCGATCACTC |
| B4 | | | 016116.3 | | CCAGTAGCACCAGAAGACATTCCGAGGA | | AATCCACATTGGCCATTTCACAAGC |
| | | | | | AACAAGTGGGAGGTTTGTGAAGAGGTTT | | CACGTGAAGAGGGGTTTTCCCATTG |
| | | | | | A | | |
| AT | PT | | NM_ | 29 | GAGCAAGCCAGGTAGCTGAAGTCTTGTC | 30 | CGAAAGCCATGACCTCCGATCACTC |
| P1 | | | 000701.7 | | AAAAGAGACACCACTCTGATTCCAATAT | | CTGGTTAGCCTGAAACACTGCCCTG |
| A1 | | | | | GCCAATTGAGGCGAGGGAACTGGGATT | | TTACAAAGACCTGCAATTCTGGACA |
| | | | | | GT | | |
| AT | CD | | NM_ | 31 | TCCATGTTCACCCCCATGGCTGCAAAGA | 32 | CGAAAGCCATGACCTCCGATCACTC |
| P6 | | | 001692.3 | | CGATGGCGAAGTTGTCGCTTACAGATCG | | CCATGGTTCCATTCTGCTCAAAGTC |
| V1 | | | | | TGTGCTCATGACTTCCACAGACGT | | AGACTTGAAGAATCTGGCTGTC |
| B1 | | | | | | | |
| AT | CD | | NM_ | 33 | GGGCTCACGTCAGGAAGGCGCAGGAAT | 34 | CGAAAGCCATGACCTCCGATCACTC |
| P6 | | | 001693.3 | | TCAGAAAACTCTAAAGCTTGGAACACTC | | CACAGAGAGACCAACAGGACGCAT |
| V1 | | | | | ACTAATTGTCCGTGCGACGACAAATTAC | | GGCAGAACTGCATAGAAGATCGCTC |
| B2 | | | | | CG | | A |
| AV | CD | | NM_ | 35 | GTGACATAGGTGCGACGGCCCCAGGGC | 36 | CGAAAGCCATGACCTCCGATCACTC |
| PR | | | 000054.2 | | TCCGCAAACCAATTTGGTTTTACTCCCC | | GGGTAGGTGCCACGAACACCATCA |
| 2 | | | | | TCGATTATGCGGAGT | | GGGCAATCCAG |
| B2 | HK | | NM_ | 37 | AGACAAGTCTGAATGCTCCACTTTTTCA | 38 | CGAAAGCCATGACCTCCGATCACTC |
| M | | | 004048.2 | | ATTCTCTCTCCATTCTTCAGTACCACGC | | GGGGTGAATTCAGTGTAGTACAAGA |
| | | | | | GATGACGTTCGTCAAGAGTCGCATAATC | | GATAGAAAGACCAGTCCTTGCTGAA |
| | | | | | T | | |
| BM | UR | | NM_ | 39 | TCTTCCTCCTCCTCCCCAGACTGAAGCC | 40 | CGAAAGCCATGACCTCCGATCACTC |
| P4 | | | 001202.3 | | GGTAAAGATCCTAGCTGTTATGGCTATT | | GCGCTCAGGATACTCAAGACCAGT |
| | | | | | GCTGAAACAGCAAAATT | | GCTGTGGATCTGC |
| BN | PT | | NM_ | 41 | ACTCCTCTGTTAGGATGAAAACGAGGTG | 42 | CGAAAGCCATGACCTCCGATCACTC |
| C2 | | | 017637.5 | | TTACCACTGGCCTTTTCTATAGCCGAGA | | ATGAGTAATGATCAAGAAAGGAACG |
| | | | | | TACGTCTTACCGCACATAGGTAACCTAT | | ATCCATGCTGGTGCCATTCGCACCT |
| | | | | | T | | |
| BS | DT | | NM_ | 43 | CTGAAATCGCTGCAACTCAAGGCTTCAA | 44 | CGAAAGCCATGACCTCCGATCACTC |
| ND | | | 057176.2 | | GGGAGAGCAAGATCCCTTTCAACAGTTT | | CTGTAGTTCAGCCTAAACACCGAGA |
| | | | | | CATGCTAATCAAAGAAGAGCTAATCCGA | | CAGGCTTACACAGGGAGAGAAGAC |
| | | | | | C | | A |
| CA | DT | | NM_ | 45 | CCCCAGCACAGAGAATAAGAGCAAGATC | 46 | CGAAAGCCATGACCTCCGATCACTC |
| LB | | | 0049 | | CGTTCGGTACAGCTTCCCCTTGACGTAG | | TAGATACAGTGTATCACTAGCAAGT |
| 1 | | | 29.2 | | ATTGCTATCAGGTTACGATGACTGC | | GGTTGCGGCCACCAACTCTAGTTAT |
| CD | UB- | | NM_ | 47 | CTCTTCTGTCTTCTGAGGCCAGGAGAGG | 48 | CGAAAGCCATGACCTCCGATCACTC |
| H1 | CD- | | 004360.2 | | AGTTGGGAAATGTGAGCAATTCCATAAA | | ATGGGCCTTTTTCATTTTCTGGGCA |
| | DT | | | | ATTGGTTTTGCCTTTCAGCAATTCAACT | | GCTGATGGGAGGAATAACCCAGTCT |
| | | | | | T | | |
| CD | X | | NM | 49 | ACATCTGGGCATTGTGGCTGACCACCAC | 50 | CGAAAGCCATGACCTCCGATCACTC |
| H1 | | | 004062.2 | | GGGGATTATGTGTTCACGCTCGTAACCC | | CACGTTGCAGCGACACACGATCACT |
| 6 | | | | | CGGGCACTAGATTATGGTGAATGGT | | CGAACCAGGAGCTGCC |
| CD | SC- | | NM_ | 51 | TATGATGTCTACCCTGTTCTCAGGAACT | 52 | CGAAAGCCATGACCTCCGATCACTC |
| H2 | PT | | 001792.3 | | TCACCATACTATTTCTGTTCACGGATGA | | GGGTTGATCCTTATCGGTCACAGTT |
| | | | | | AGGCCTATATCAATG | | AGATTAGCTAC |
| CD | PT | | NM_ | 53 | AGTTGGCTGACCTGACATGGAGTGTAGT | 54 | CGAAAGCCATGACCTCCGATCACTC |
| H6 | | | 004932.3 | | AAAACACCAGGACAGCGGAAGCCGGCT | | TTAAAGCACAGTCCTTTATCCCATT |
| | | | | | ATGATTCACCACCGTGTAGGAGTTATGC | | AGCCTGTGAAATGTACAGTTAGGGC |
| | | | | | GC | | |
| CL | DT | | NM_ | 55 | AGAGAGTCTCCCCAAAGAGGCGCCCGA | 56 | CGAAAGCCATGACCTCCGATCACTC |
| Cn | | | 0011 | | TAGCAGCTCCATAGACAAAGATGCGGAT | | GATGGGATTGGTGATCCCTCCAGC |
| KB | | | 65945.1 | | GTGTCGCTAACCTCCTGGTCCTATAAAG | | CACGATGCCCTCAGGGAAGATAAAA |
| | | | | | CA | | G |
| CL | LH | | NM_ | 57 | AACTCAAGAGGCAGCTTGCCAGCGAGC | 58 | CGAAAGCCATGACCTCCGATCACTC |
| DN | | | 0011 | | TCTTTTAGACATAAGCATTTTTACAGAA | | ATGGGAGGGCCTTGATGGGATCATT |
| 10 | | | 60100.1 | | ATGTCACTCCCATGGTGGCTGATATAGA | | TTGTGAACAGTTCGCTTTTATAACA |
| | | | | | AA | | |
| CL | LH | | NM_ | 59 | AAAAATTGCCCAGAAGTAAACTTTGTGC | 60 | CGAAAGCCATGACCTCCGATCACTC |
| DN | | | 0011 | | TGGAACCCCTGCCTCCATTGACCATGTC | | CCCTGTGCTCTAAAGACATTTCCTC |
| 14 | | | 46077.1 | | GAACCTTGGATAGGAGCGACCGATTACG | | GCATTCACATTATTTCCTTGGATAC |
| | | | | | T | | |
| CO | IJ- | | NM_ | 61 | GCCCTGCGGCACAAGGGATTGACACGC | 62 | CGAAAGCCATGACCTCCGATCACTC |
| L1 | MX | | 000088.3 | | GTTCCCCAAATCCGATGTTTCTGCTCTG | | GTCTTTCAGCAACACAGTTACACAA |
| A1 | | | | | TGAACTGTCATCGGTCCGATCAATTAGT | | GGAACAGAACAGTCTCTCCCGCCC |
| | | | | | CT | | A |
| CO | SC- | | NM_ | 63 | CCTCTCTTTCCTTCTTCACCACTGGGAC | 64 | CGAAAGCCATGACCTCCGATCACTC |
| L1 | MX | | 000089.3 | | CAGGAGGACCTTGCCTATGCATCATGTG | | CTGGAGGGCCGGCAGATCCAGCTT |
| A2 | | | | | CCTCACTAGGACATCATGCT | | CCCCATTAGGG |
| CO | MX | | NM_ | 65 | GACCCCTTGGTCACCCTTGTCACCTTTT | 66 | CGAAAGCCATGACCTCCGATCACTC |
| L4 | | | 001845.4 | | GGTCCTTGAAAACTTAAGCCCACGGGAA | | TCTCCTTTTTCTTGAACTTGAGCTT |
| A1 | | | | | AACCATTGATCATGTATTCTTTGTCGCA | | GTCCTGGTACTCCTGGAGGCCCACT |
| | | | | | T | | |
| CO | MX | | NM_ | 67 | CTCAGTCCAGGAAAGCCCATGATTCCTT | 68 | CGAAAGCCATGACCTCCGATCACTC |
| L | | | 001846.2 | | CTTCTCCCTTCAAGGAAATGCCCCCTAT | | CTTCTGTCCTGGTGATCCTTTTTCA |
| A2 | | | | | GTTGTTGTTGAGGAGATTTATGTTGTGG | | CCACTCAAGCCAGGGTAACCC |
| | | | | | G | | |
| CO | MX | | NM_ | 69 | CTTTGACCCAGTGGTCCCATTTCACCTT | 70 | CGAAAGCCATGACCTCCGATCACTC |
| L4 | | | 000091.4 | | GATGACCTGGAATTCCCTCTAATACATC | | CATCTTCACCTGGTGGCCCTAAAAT |
| A3 | | | | | GTGATACGGGCGATATAATGCTC | | TCCCGGATTTCCTGGATATCCT |
| CO | MX | | NM_ | 71 | AGTTCCTTTCTGACCTTTCAATCCATGC | 72 | CGAAAGCCATGACCTCCGATCACTC |
| L4 | | | 000092.4 | | AAGCCGTTCAGGCCGAATTTGTCCGCTG | | TGGACCAGGTGGCCCCACATCATG |
| A4 | | | | | GGTAATGGTGGAATTAAAGA | | CAAACCTGAAGCACCTTT |
| CO | MX | | NM_ | 73 | AGAAGCACAGGGAAAAGAATTGTTAATT | 74 | CGAAAGCCATGACCTCCGATCACTC |
| L4 | | | 000495.4 | | CTGCGTGTGTGTTTTTGTTTCTCTTTAC | | GTTGTTTCCCAGTTCTGTACAAGCA |
| A5 | | | | | TTGTTGTCACCCCAGAGATGTTCTCAAT | | GAGGCCACAGGAGGATTCTTACATA |
| | | | | | T | | |
| CO | MX | | NM_ | 75 | ACCCTTTTGACCCTCAATTCCTTGGAAT | 76 | CGAAAGCCATGACCTCCGATCACTC |
| L4 | | | 001847.2 | | CCATTAAGCCCAGGAAATCCCACTCGTC | | ACAGCACCATCTATATCGATGAAAA |
| A6 | | | | | AATGATCTACGATCCTCGCTAAGTATTC | | CATCTGGGCCTGGCAGGCCAATGT |
| | | | | | A | | C |
| CR | N | | NM_ | 77 | CTGGTGCAGACCACGTCATCGGCGCCA | 78 | CGAAAGCCATGACCTCCGATCACTC |
| AB | | | 004378.2 | | AACGTCAGGATAAGTTCATCGCTTCAGT | | CTTCCTGAAAGTAGGAGCAAGCCAG |
| P1 | | | | | TAAAGGCTATCTTGCTCCGCTCGTTCTC | | CTGCCTTCACTCTCGGACATAAATT |
| CU | PT | | NM_ | 79 | GAGACGAATCTCAGCGTCAGGGCGCTG | 80 | CGAAAGCCATGACCTCCGATCACTC |
| BN | | | 001081.3 | | CTGAAGGATGTGATAGGATGGGGCCAC | | CTGATGCGGTGACCGTGGTGTGGA |
| | | | | | CGTTTTGCCAGTTCCACCAGTAACTGTA | | AACCCCCAGCACTGATGCTAGAATC |
| | | | | | AGG | | A |
| DB | CD- | | NM_ | 81 | GCTTCGTTTGCGTCGTTCGTGGCTGGAA | 82 | CGAAAGCCATGACCTCCGATCACTC |
| A | DT | | 001022.3 | | GGAACAGTGGTGGAGAATACTACCGGC | | GTGTAATAGAGAGAGGATAGAGAG |
| | | | | | ACAAGCAGACAAAATCAACATGGTCATT | | CTCCTGTTCGGAGCTGGGGGAACT |
| | | | | | TA | | TG |
| DU | LH | | NM_ | 83 | CCCCCACCCCCATCCCGCAAAGAAAAAG | 84 | CGAAAGCCATGACCTCCGATCACTC |
| SP | | | 001395.2 | | TAACAGTCGCAGAAGAGGCAGGCAGTT | | CCGGGACATGGGCCTGGACAACCA |
| 9 | | | | | CTTAATTCCATTGGCTCTCTCAATGGGT | | CCTGGAGAGGGAA |
| | | | | | CG | | |
| EB | SC | | NM_ | 85 | AGTCTCAAAGGAGATTCTCACACAGTAT | 86 | CGAAAGCCATGACCTCCGATCACTC |
| F1 | | | 024007.4 | | GTGTGCCACCAAGCTCTCGAGACAGTG | | CAGAGTGTGGAATTCTGTGCCTGGA |
| | | | | | CGTTTTATAAATACAGGGATGCTGTTTT | | CCCTGTTCCATGA |
| | | | | | TC | | |
| EG | X | | NM_ | 87 | TATTTTCTCTGATGTCTCCAACAGAGCC | 88 | CGAAAGCCATGACCTCCGATCACTC |
| F | | | 001963.4 | | TTCACTCCCGATATTTTGCTGATGTAAC | | CAGCCGCTTATCAAGCACATCCAAT |
| | | | | | TGTTACTGACCTTGC | | GACACAGCTGT |
| EL | CD | | NM_ | 89 | GAGCCCAAGCACTGAGCAGGGAGGTGA | 90 | CGAAAGCCATGACCTCCGATCACTC |
| F3 | | | 004433.4 | | ACGCCAGTCCAGAAAGAAGCTGTGTCC | | ACATATTTATATAGCACATAAATTA |
| | | | | | GTCTATACGCATACTGGTCCACATATA | | GGGAGTGCTCTGACCCCTGCCCGTG |
| EM | UB | | NM_ | 91 | AGAAGTTTGCATCAGGGTTTTAAGCCTC | 92 | CGAAAGCCATGACCTCCGATCACTC |
| X2 | | | 004098.3 | | AGGATGTTAGGAAAGGGAATGTCACCGT | | GCATTGCAGTGAATGGTTGAACTCG |
| | | | | | GTGGACGGCAACTCAGAGATAACGCATA | | GCCAATTTCTCCAACCACTGAAAGG |
| | | | | | T | | |
| EN | N | | NM_ | 93 | GCAAAGCACAAGTGACACATGGTCCCTC | 94 | CGAAAGCCATGACCTCCGATCACTC |
| O2 | | | 001975.2 | | TCTAACACCTCAGCACACCAACCCCGAA | | TATGCACAGTTCACGGCTCATATGC |
| | | | | | TGTATAATGCTGACGTTCTTGCTTTTGG | | AAAGTGGAAGACACGTGGGACAAG |
| | | | | | C | | A |
| ET | UB | | NM_ | 95 | TTCCATCAGGTTGTCTGGTGCAGAAAGC | 96 | CGAAAGCCATGACCTCCGATCACTC |
| V5 | | | 004454.2 | | ACAAAGCAGGCAACAAGGCGGGCCTCA | | TGTCCAGGCTGCCCTGCACACTTCA |
| | | | | | AGACCTAAGCGACAGCGTGACCTTGTTT | | AAAATGTACAACTCAGGTGCAAATG |
| | | | | | CA | | |
| FG | MX | | NM_ | 97 | ATTGCTATTCACAGTCTCATCTATATAT | 98 | CGAAAGCCATGACCTCCGATCACTC |
| B | | | 005141.5 | | AATTGGTGCTTTTCCAGTTCTGCGCTTT | | CTTCTCAGGTTTTCCAGGATTGAAC |
| | | | | | AGAATGCTTAAAGATGGCAGAGTTGGAG | | GAAGCACACGAAGGTTAGTTGGGAT |
| | | | | | G | | |
| FG | MET | | NM_ | 99 | CCAGCCTGCAGAGCAGCGCACAGCTCA | 100 | CGAAAGCCATGACCTCCGATCACTC |
| F8 | | | 033163.3 | | TCTTGCTTGAGCTGATGTACAGGGTGAA | | AACAGCAAACAATATCAACAACCGG |
| | | | | | GGAACTGTAACGGCAGCT | | AACCCAGGGCTCCCCCAGCACCTC |
| | | | | | | | C |
| FN1 | MX | | NM_ | 101 | TCGATGTGGTCTGCACAGAGGTGTGCCT | 102 | CGAAAGCCATGACCTCCGATCACTC |
| | | | 212482.2 | | CTCACACCTCCCCTTTCCCAAGTAAATG | | CTGCGGTTGGTAAACAGCTGCACG |
| | | | | | TACGGGAATTATCG | | AACATCGGTGAAGGGGCCAGATCC |
| | | | | | | | GC |
| FO | SC | | NM_ | 103 | GCGCCTGGAGGAGCGAACAAAACACCG | 104 | CGAAAGCCATGACCTCCGATCACTC |
| XD | | | 004472.2 | | AACCACCAAGACGAGAAAAGGAGCACAA | | TCCGAGAATTCGCAGCGGCGAAAAT |
| 1 | | | | | ACTCACTACTACCAACAACCTCACCAAA | | GGGCGCGCGAGGTCGAGAGGGGC |
| | | | | | AA | | C |
| FO | UB- | | NM_ | 105 | AAGCGTTGAAAGGCGGACGGAGGCTGG | 106 | CGAAAGCCATGACCTCCGATCACTC |
| XI1 | CD | | 005250.2 | | AGGAGGCGGCCAGGAGCGCCTGCCAAT | | CGAGCTGGTAAAAGCCGCCCTGGA |
| | | | | | GCACTCGATCTTGTCATTTTTTTGCG | | CATGCGGGTCGAGCATCAGGG |
| FO | CD- | | NM_ | 107 | ATGTCTCTCACACTCAGTCATACCTGGA | 108 | CGAAAGCCATGACCTCCGATCACTC |
| XQ | DT | | 033260.3 | | AATGCCACATACGTACACGGATCTCTAA | | GCACAGTGAAGCATCGGTCAATAGC |
| 1 | | | | | GGTAAAGTGCTTCTCAATAACATCCGCT | | AAGAAATCACTGAAGAGCCTCTGAC |
| | | | | | C | | |
| FX | DT | | NM_ | 109 | AGGCCCCCATTGCGAACGGTCTCATAGT | 110 | CGAAAGCCATGACCTCCGATCACTC |
| YD | | | 021603.3 | | CATAGTAGAACGGGTCCCAATCAGAAAC | | AGGAGGATGAGGAGCCCCACGATG |
| 2 | | | | | CCTACTTGCCTGCTGCATGTTGTG | | AAGGCCAGTCCAGCGAAGATC |
| GA | HK | | NM_ | 111 | TCCCCTCTTCAAGGGGTCTACATGGCAA | 112 | CGAAAGCCATGACCTCCGATCACTC |
| PD | | | 0012 | | CTGTGAGGAGGCTAGGACGCAAATCACT | | TACTTTATTGATGGTACATGACAAG |
| H | | | 56799.1 | | TGAAGAAGTGAAAGCGAG | | GTGCGGCTCCCTAGGCCCC |
| GA | SC | | NM_ | 113 | CGACTGCCCGCCCATATTGCACTTGGTC | 114 | CGAAAGCCATGACCTCCGATCACTC |
| TA | | | 0011 | | ACTACATCTATTTGTGGCAGAATATGTC | | GACACTAGTACAAAATAAGTTACTT |
| 2 | | | 45661.1 | | GATGCAAAAAACCT | | CTGGCCGTGGTGCTCCCTGCAG |
| GA | UB- | | NM_ | 115 | TTTCCTTTGCAGAGGCCAACAGCTTTGA | 116 | CGAAAGCCATGACCTCCGATCACTC |
| TA | CD- | | 0010 | | ACAAATGATTCGCCTACAACTTCTTTCG | | ACCCTCAACGGCAACTGGTGAACG |
| 3 | DT | | 02295.1 | | GGTTATATCTATCATTTACTTGACACCC | | GTAACACTGATTGCCCAGAACTGGT |
| | | | | | T | | A |
| GD | X | | NM_ | 117 | AAGTTTTTCAATATTTTGTCGTACGTTG | 118 | CGAAAGCCATGACCTCCGATCACTC |
| NF | | | 000514.3 | | TCTCAGCTGCATCGCAAGAGCCCGCTTT | | CCTACTTTGTCACTCACCAGCCTTC |
| | | | | | ATTATGTGTTCGTCTAACTCTGTTTCTG | | TATTTCTGGAT |
| | | | | | T | | |
| GF | UB | | NM_ | 119 | GCACACGGTGGCAAAACATGAGTGGGA | 120 | CGAAAGCCATGACCTCCGATCACTC |
| RA | | | 005264.4 | | ATTTCATTCTCAGACCCTGCTGGCTTCC | | TGTGTATTGCCCGACACATTGGATT |
| 1 | | | | | TTCCTGTGTTCCAGCTACAAACTTAGAA | | TCAGCTTCTGTGCCTGTAAATTT |
| | | | | | AC | | |
| GJ | X | | NM_ | 121 | TTGCCTGGGCACCACTCTTTTGCTTAAA | 122 | CGAAAGCCATGACCTCCGATCACTC |
| A1 | | | 000165.4 | | AGTAGTGAAGTCACGCCAAGTGCTAGCA | | TTGTCAAGGAGTTTGCCTAAGGCGC |
| | | | | | TCTCTGACGAAAACAGCAGACGGAAAAG | | TCCAGTCACCCATG |
| | | | | | T | | |
| GP | LH | | NM_ | 123 | ATCTGAGCCTGTTATGAAAGGTGTAGCT | 124 | CGAAAGCCATGACCTCCGATCACTC |
| D1 | | | 005276.2 | | CTGTCAGCAGATTAAGTTCCTGCATGAA | | CTACTGAGAAGGAAGCCTGGGTGA |
| | | | | | CGTGTCGTGTTATGCAGCGGTATGTCGT | | ATACAAAGGTCACAGGGAGCTGAGT |
| | | | | | G | | G |
| H2 | Y | | NM_ | 125 | CTAGGTGCTTGGATTGCCGAGTTGAGTT | 126 | CGAAAGCCATGACCTCCGATCACTC |
| AF | | | 002105.2 | | TGCTGGAAGGGAAATGGGGCGGCCTAA | | CAACGGAGGCTCAGTCCAGACAGG |
| X | | | | | ATTGGGAAAAAAGGTTTTAGCTATTGAT | | GATTAACCGACTTGTGCTGGTAT |
| | | | | | GG | | |
| HA | IJ | | NM_ | 127 | TGATTGTTATTCCAAAGGCCATCTGAAG | 128 | CGAAAGCCATGACCTCCGATCACTC |
| VC | | | 0010 | | ACTCTGTCACGGTGTCATTCCCCCTACA | | CTTTAGTGGTATTGGCCGTCAGTAG |
| R1 | | | 99414.1 | | TATATAGGAAAAGGGAAGGTAGAAGAGC | | ACTATGTTCTAGGAACAGTTGAGTT |
| | | | | | T | | |
| HG | X | | NM_ | 129 | GCAGTATTCACTTTTTTGGTTTTTATCT | 130 | CGAAAGCCATGACCTCCGATCACTC |
| F | | | 000601.4 | | TCAGTGCTGGATCTATTTTGATCCTGTT | | AAGTGAATGGAAGTCCTTTATTCCT |
| | | | | | GCAGTATCACGTAAATACCTACTTCGAT | | AGTACATCTATTAGCACATTGGTCT |
| | | | | | A | | |
| HI | SC | | NM_ | 131 | CGACTCCAGAACTCCCCGCCCTTGGTGA | 132 | CGAAAGCCATGACCTCCGATCACTC |
| C1 | | | 006497.4 | | CTGAGGAAGGAAGGGTTGGCCCCTCAG | | CGCGGGAGTGGGAGACCTGGTGGT |
| | | | | | CATCTTTAGCAGTAGCACTTGCTAAATT | | AGGCTCTTCGCCTTC |
| | | | | | GG | | |
| HN | PT | | NM_ | 133 | TGCATAGAAGGGAAACTGGGCTTCGGG | 134 | CGAAAGCCATGACCTCCGATCACTC |
| F1 | | | 000458.2 | | CTGCGCCTCCTGGTCAAGACTTGCATGA | | AGACAGGAGTCCTTGACATCGTGG |
| B | | | | | GGACCCGCAAATTCCT | | GAGAGGCATTGTGGCAATAC |
| HN | PT | | NM_ | 135 | GAAGCTATTCAGCCACTGTAGTTAAGAG | 136 | CGAAAGCCATGACCTCCGATCACTC |
| F4 | | | 178849.1 | | CTCCTGTTCTGATCCAGCGATTAAGCCG | | AAGCCCCTCAACTTGATCACGGTGA |
| A | | | | | TAGTTGAATTTATGGAGCGGTGCC | | GAACACAGGGAGCCTTTGGA |
| HO | SC | | NM_ | 137 | CCAGTGAAGCCACTGCTTTACACAGAAG | 138 | CGAAAGCCATGACCTCCGATCACTC |
| PX | | | 0011 | | ATACACATAGCTTCCTATTGTTCATTTG | | ACACCATCTGTCATCATGACTCAAA |
| | | | 45460.1 | | GTGTTGCCTAGTACTAGGTGACTGGTAC | | GGGAAATGCTAGCCACACCATTTTT |
| | | | | | C | | |
| HO | UB | | NM_ | 139 | TCTTTTCCCACAAGGATTTGCAGCCCTC | 140 | CGAAAGCCATGACCTCCGATCACTC |
| XA | | | 005523.5 | | TTCCACCTCAAAGCTACCTCCACCATGC | | GGTGGCTAGGAGGAGTGGAAAGAC |
| 11 | | | | | TGAAATCATGGTTGAAGAAGAAGAAGAT | | ACTGATGCCACCTGGAATCATAGAT |
| | | | | | C | | T |
| HO | SC | | NM_ | 141 | ATCAAAACCTGCCCTCTCAGACACATGC | 142 | CGAAAGCCATGACCTCCGATCACTC |
| XA | | | 000522.5 | | AGACCCAACAGCAGATTTTAAACATCAA | | CGTCAAGGTCAAGGTTTAAGGCCTT |
| 13 | | | | | CACCAAACGCAATATCAGGCTGAATGGT | | TCGTAGTCCTGTCATTTCAACAAAT |
| | | | | | G | | |
| HO | X | | NM_ | 143 | AGGGTTAGTCCAGACCCACAGTCCACAA | 144 | CGAAAGCCATGACCTCCGATCACTC |
| XB | | | 004502.3 | | AGACAGCATTAAAGAGGGCTTACCTAGT | | TGGGAAGGCGGAGGCTCAGGAGG |
| 7 | | | | | TGACTTGGAGTATGCCATGAAGACTCGT | | GCAGGCAACCAC |
| | | | | | C | | |
| IL1 | UB | | NM_ | 145 | TCAGCTTCCATCGCTGTGCAGAGGAACC | 146 | CGAAAGCCATGACCTCCGATCACTC |
| RN | | | 000577.3 | | AACCGGGGCGAGCTATTGTAAATACGCA | | CTTCGTCAGGCATATTGGTGAGGCT |
| | | | | | GCAACAAGCGTCCAG | | GACGGGCTGG |
| ISL | X | | NM_ | 147 | CAAACTTCTCTGCTCCAACTCAGCTCCA | 148 | CGAAAGCCATGACCTCCGATCACTC |
| 1 | | | 002202.2 | | TCGCCATTGGTCTGACGCAGCGCCTCG | | CGGCAGACTCGGCGCGGCTCGGG |
| | | | | | AACAGTGATACGCACACTGATAACTATG | | CACCTCTCTTCCTTATCTCTTACT |
| | | | | | CG | | |
| ITG | X | | NM_ | 149 | GCTCCTCAATGAAGTTCCGCAGCTGTAG | 150 | CGAAAGCCATGACCTCCGATCACTC |
| A8 | | | 003638.2 | | CCAAATGGCTCCCTGTTTCATTAATGTA | | CAGGAAGAACATGAACTGCACCAG |
| | | | | | ACGGACAGGTCGCTGAGGT | | CGTGCTCGTCCATGTGCCTTTCATT |
| | | | | | | | T |
| JA | PT- | | NM_ | 151 | CATCCCTCTGTCAGGCAGAGCCTATTAT | 152 | CGAAAGCCATGACCTCCGATCACTC |
| G1 | LH | | 000214.2 | | ACCCCGATTACCAGAACAGCTCTAACCT | | TGAGAACTCCAGATGAGTTTTGTGG |
| | | | | | GCATACATATGGCATTTAGTTGTTCA | | TTGAAAAGCCTTTCAGTTCTTCCTC |
| KC | DT | | NM_ | 153 | CCTTTGTCTTGGATACTATGGGAGCAAA | 154 | CGAAAGCCATGACCTCCGATCACTC |
| NJ | | | 153766.1 | | ACGGTAGCCCCAAAGCACCTCCCGATAC | | CACTTCCACTGTCTTGCTAAAGTTA |
| 1 | | | | | CCGTAGCGTGATGGTCTTATAGCTGCTC | | TGGAAATCCACTCGGTATTTCCCTT |
| | | | | | T | | |
| KD | V | | NM_ | 155 | CACTCACTTCCATAATCGTCAGTACATG | 156 | CGAAAGCCATGACCTCCGATCACTC |
| R | | | 002253.2 | | CCCCGCTTTAATTGTGTGATTGCATACG | | CTTTGAAATGGGATTGGTAAGGATG |
| | | | | | AAATTTGAGCAAGCAATTGAAGGCTTAG | | ACAGTGTAATTTCCTGTGTCTCTTT |
| | | | | | A | | |
| Ki6 | X | | NM_ | 157 | CTGATGGCATTAGATTCCTGCACGCTAA | 158 | CGAAAGCCATGACCTCCGATCACTC |
| 7 | | | 002417.2 | | GAGTTCTCCCTCTACATCTGCTGTAACG | | GTCTTTCTCTTCACCTACTGATGGT |
| | | | | | GTAGATGGGAAAAAGTGTGCTCATTTT | | TTAGGCGTGTGCATGGCTTTGCCTG |
| KL | X | | NM_ | 159 | TTTGGATTCCTCATTTTTCCTGATTATC | 160 | CGAAAGCCATGACCTCCGATCACTC |
| F4 | | | 004235.4 | | ACCTCACAAGATGACTCAGTTGCCTACG | | AATGATAGAAGATCCAGTCACAGAC |
| | | | | | TATATATCCAAGTGGTTATGTCCGACGG | | CCCATCTGTTCTTTGATTTTTGTCT |
| | | | | | C | | |
| KL | S | | NM_ | 161 | AATTGCCAGTTTAGAAGCAATTGTAGCA | 162 | CGAAAGCCATGACCTCCGATCACTC |
| F5 | | | 001730.5 | | GCATAGGATGGAGGTGGGGTTACATGTT | | TGGATGTTTTGTGAGTTAACTGGCA |
| | | | | | GGAGTTAACGGAGACCCGCCATCGTTTA | | GGGTGGTGGGTAAATTTGGATTGTG |
| | | | | | C | | |
| KR | CD | | NM_ | 163 | GGCCAACCGGGCATGGACACGGACAGC | 164 | CGAAAGCCATGACCTCCGATCACTC |
| T1 | | | 199187.1 | | AGGTGTTGTTGCGGTTGTTAATATGACA | | TGGAGCGAGTGGTGAAGCTCATGC |
| 8 | | | | | GGCCGCTAAAGACGTTCT | | CCCCAGAAACGGGGT |
| KR | UR | | NM_ | 165 | CTTGACTGGCGAGACATGGTGGCTTGTT | 166 | CGAAAGCCATGACCTCCGATCACTC |
| T5 | | | 000424.2 | | CCTGGTGGAGCAAGAGAACCAGCCTTCT | | TGAAGCTACGACTGCCCCCGCTCC |
| | | | | | TGAAGACCTATGTAAAGAAACGGGTCAC | | GGAAGGACACA |
| | | | | | T | | |
| KR | UB- | | NM_ | 167 | CTTCCCATCACGTGTCTCGATCTTCTTCA | 168 | CGAAAGCCATGACCTCCGATCACTC |
| T8 | CD | | 002273.3 | | CAACCACGGCCCGAACCTAACTCCTCGC | | CTGTTCACTTGGGCAGGACGTCAGA |
| | | | | | TACATTCCTATTGTTTTC | | GGACTCAGACACCAG |
| LA | MX | | NM_ | 169 | AATAAATCTTCAGCAGCCTTGAGTTCAA | 170 | CGAAAGCCATGACCTCCGATCACTC |
| MA | | | 005559.2 | | GGGTGGCATTTTGGTGCAACTGCAATCT | | ATACCTCCAATTCTTCCAGCGGCTT |
| 1 | | | | | ATATTCCTTACACGCTTGCTTGCTCCTAC | | CTGGTAATTTTCCTGAATTTGTGAC |
| LA | MX | | NM_ | 171 | CTGTGCTGATGCTCAAGTCATTACCCTC | 172 | CGAAAGCCATGACCTCCGATCACTC |
| MA | | | 000426.3 | | TAAGATAATCATAAGCTGGAGACTTTGA | | CAATACATTAGTATGTTCTTCAGAT |
| 2 | | | | | CAGAGAGTCAAAATCAATAAACTCCGAT | | GGGTGCAGGTACACCTCATCTTGGG |
| | | | | | C | | |
| LA | MX | | NM_ | 173 | CTCAAAGCCATTTCTCCGCTGACATCCA | 174 | CGAAAGCCATGACCTCCGATCACTC |
| MA | | | 0011 | | GTAGTGCTCTTCCAATTAAAGAACATGC | | AGAGGAGCCACAGAGGCAGAACCG |
| 4 | | | 05209.1 | | GTAAAACGACCTCGTTGTA | | AGCGCCAGGCTGAG |
| LA | MX | | NM_ | 175 | GAGGGAAGCAGCAGCGTTTCGGCAGAA | 176 | CGAAAGCCATGACCTCCGATCACTC |
| MA | | | 005560.4 | | CAGGGATGAGCTGCTGCCGGTGCCTGA | | CGTGGCAGCCACATGGACGGGCTC |
| 5 | | | | | CTAATCGTGTAGGTGTCTAATACA | | CGTTGTTATAGAAGAGGGA |
| LA | MX | | NM_ | 177 | GCATGAGCACTGGCCTGACTCCGCAAA | 178 | CGAAAGCCATGACCTCCGATCACTC |
| MB | | | 002291.3 | | GCAACTGTTGTTTAAGGCTCCGCCGCGT | | TAGTAACCAGGTTCCACTTCGTTGC |
| 1 | | | | | TTCGTTGCGATAACCTCATATAGTCCC | | ACTGACGTCCAATCATGTGAGGCCG |
| LA | MX | | NM_ | 179 | AAATATCAAGTACCTGGCATGTGGTTGC | 180 | CGAAAGCCATGACCTCCGATCACTC |
| MB | | | 002292.3 | | AGAGTCCCTTGGATGCGATCATCGAGCT | | TTCAGATGCAGCTTGTAGGAGATAC |
| 2 | | | | | TGGTTCTGTCCCTATAGCTACAGCTGTC | | CAGGCTCAAGGCAGACAGGATTAG |
| | | | | | T | | G |
| LA | MX | | NM_ | 181 | AACTTCACCCAAACTAGCTTGGCAGTCA | 182 | CGAAAGCCATGACCTCCGATCACTC |
| MC | | | 002293.3 | | ATCTTCAGTCCTCTTCTGCAACCCGTAG | | AAGGAAACCAAACCAAACCACCCCA |
| 1 | | | | | GAGTCGGTTGCCACTCGTTGATAGTCAA | | TTGTGGCCTGAGACTTGCTGGAGTG |
| | | | | | C | | |
| LA | MX | | NM_ | 183 | TGTGCCGGTAAAAGCCATTCTTGCACTT | 184 | CGAAAGCCATGACCTCCGATCACTC |
| MC | | | 005562.2 | | CTCGCAGTGAATGCCATCAGTGCTTACT | | AAGAGAACCTTTGGAGTTACAATTG |
| 2 | | | | | CTTGCCAGTGGGCATGAAATTGCGAGCT | | CAGGGCAAACAGCGGTCCCTTTCTC |
| | | | | | T | | |
| LA | MX | | NM_ | 185 | CGTAAGCTGCTGACTGGATGGATTAGAC | 186 | CGAAAGCCATGACCTCCGATCACTC |
| MC | | | 006059.3 | | TTGAACACGTGTGGGTCATACACTAATG | | CACACAGGTGGCTCTCACAACAGCT |
| 3 | | | | | ATCCCGCGGTGTAGTAATTCTACGGAAT | | GTGGACTGTAATGTGTGTGGAC |
| | | | | | G | | |
| LH | MET | | NM_ | 187 | ACACACTCGGCCGTGCAAGCCAAAACTC | 188 | CGAAAGCCATGACCTCCGATCACTC |
| X1 | | | 005568.3 | | GCACCAGGAAATCAAAGTAGGGCTGAG | | ACTCCTGAAGACAATCGGCACGAG |
| | | | | | GCTGTTAAAGCTGTAGCAACTCTTCCAC | | CTCCCCAGTCTCTCCAAAAAGAGGA |
| | | | | | GA | | C |
| LR | PT | | NM_ | 189 | AGCACCAAACCTAGAGCCCTCCCCTCGC | 190 | CGAAAGCCATGACCTCCGATCACTC |
| P2 | | | 004525.2 | | ACAGTGTAATACACAACCAGCTCGGATT | | ACAAGATTATTGCGGCCGGATTCAA |
| | | | | | GGATGTCTCGTAAGGAATACTTAT | | AGTTGGGGATGTAGGCACGTTTGAT |
| MA | N | | NM_ | 191 | GGTTTCTTGTATGGCAGTTACACTGGTT | 192 | CGAAAGCCATGACCTCCGATCACTC |
| P2 | | | 002374.3 | | GCCATATCCACTGGGTCACCAACTTAAA | | CCTGTGTAAAATCTCTTAGCTGAGA |
| | | | | | GCTATCCACGAATGTCAAAAATGTGGTT | | ATGAGTGGTGTGGGTTTGCTCCTAG |
| | | | | | T | | |
| MC | V | | NM_ | 193 | AAAATGTCACAGGAGACTTTGAAGAGGC | 194 | CGAAAGCCATGACCTCCGATCACTC |
| AM | | | 006500.2 | | AGACAGGAGGGTGGTGGCAGCACTATC | | GCACGTATCTTTTAAGGAATGACAC |
| | | | | | AATTCGTGACCCCGATCATCCAGTCCAG | | CAGTTCCTGGCTTCTGACCAAAGAA |
| | | | | | AA | | |
| ME | SC | | NM_ | 195 | CCTATGAAGAATTAGGGATGCTCCTTGG | 196 | CGAAAGCCATGACCTCCGATCACTC |
| IS1 | | | 002398.2 | | TCCAGAGTAGATGCCAAGAATGCATTCG | | GCATGTCCCCCAAATTGACTTCAGT |
| | | | | | CAACCATGTGAAGTAATGTGAGCGTACT | | TGGTATTTCCTGCTTTTTAAAGGTC |
| | | | | | T | | |
| ME | SC | | NM_ | 197 | AGAGACCAGCTTAACTCAGAGCACACTC | 198 | CGAAAGCCATGACCTCCGATCACTC |
| IS2 | | | 002399.2 | | CCTCCAAGCGGTGCACCAGTTAGCGTG | | CTGCGCAGATCCAGCAACCTTGTCA |
| | | | | | GCGTATACCATGTTGTTAACA | | ATACTCATTTTTTTCCAGGACCAGA |
| MM | CD | | NM_ | 199 | AGAGTCCAAGAGAATGGCCGAGTTCATG | 200 | CGAAAGCCATGACCTCCGATCACTC |
| P1 | | | 002421.2 | | AGCCGCAACACGATGTAAGTTGCTATGA | | ACTGAAGGTGTAGCTAGGGTACATC |
| | | | | | TAAATGGTTTTCATCAGCCGGATTTTGT | | AAAGCCCCGATATCAGTAGAATGGG |
| | | | | | T | | |
| MM | UB | | NM_ | 201 | AATGCTGATTAGCTGTAGAGCTGAAGGC | 202 | CGAAAGCCATGACCTCCGATCACTC |
| P2 | | | 004530.2 | | ACGGCTGCCAGGCAGCAAGAAGGAGTA | | CACCGGGAGGAGCCACTCTCTGGA |
| | | | | | TGGAACTTATAGCAAGAGAG | | ATCTTAAATTACCAGGTAGGAGTGA |
| | | | | | | | G |
| MM | UB | | NM_ | 203 | GCACTCCACATCTGGGCTTCTGCATTAT | 204 | CGAAAGCCATGACCTCCGATCACTC |
| P7 | | | 002423.3 | | TTCTATGACGCGGGAGTTTAACCTATCA | | GGAAGTCCATTTTGGGCTATTTGGA |
| | | | | | GCTAATAGGGTCGGCTCAACAGTGTATC | | AATAGTGAGTATTCTGCAACATCTG |
| | | | | | C | | |
| MU | UB | | NM_ | 205 | GGAGATGCAGACACTGATGCAGTCGTG | 206 | CGAAAGCCATGACCTCCGATCACTC |
| C6 | | | 005961.2 | | GGATGAGTGGACAATGAGGAGTGCTCA | | GGAGAGTGGCCCTAATGGTAGTAG |
| | | | | | AGAGCTTGCGAACAAGACGGAAGAAGAT | | AGGCAGCTGGAGAAGAAGGAAAAA |
| | | | | | ATG | | GA |
| MY | S | | NM_ | 207 | CGCCCGCTGCTATGGGCAAAGTTTCGTG | 208 | CGAAAGCCATGACCTCCGATCACTC |
| C | | | 002467.3 | | GATGCGGCAAGGGTTGCGCTATGCAGA | | CGTCCTTGCTCGGGTGTTGTAAGTT |
| | | | | | CGAGCTGGCAGAGGAGAGAAATCA | | CCAGTGCAAAGTGCC |
| MY | SC | | NM_ | 209 | AATGTCCTCTCGTCTCTGGCTTGGCGAA | 210 | CGAAAGCCATGACCTCCGATCACTC |
| H1 | | | 0010 | | TTGCCCGTGATTTTTCTAGCAGCGTTGG | | CACTTCTCATCTTCTCCTTGGCTCC |
| 1 | | | 40113.1 | | GAATGCCATCTCTATCCCTTCTTTTTCA | | AGCAATCATGTAGTAAAAGATGTGG |
| | | | | | A | | |
| NA | S | | NM_ | 211 | TCAGTTTCACTCATCTTCACACGTCTTC | 212 | CGAAAGCCATGACCTCCGATCACTC |
| NO | | | 024865.2 | | AGGTTGCATGTTCATGGAGTAGCACCCC | | GGAGGGGAGAGGAAGGATTCAGCC |
| G | | | | | TCCAAACGCATTCTTATTGGCAAATGGA | | AGTGTCCAGACTGAAATTGAGTAAT |
| | | | | | A | | A |
| NG | IJ | | NM_ | 213 | CTGATCCAGTAGTCACACTTCTTTTTCC | 214 | CGAAAGCCATGACCTCCGATCACTC |
| AL | | | 005564.3 | | TAAACAGGACGGAGGTGACATTCACGCA | | TAATGTTGCCCAGCGTGAACTCGCC |
| | | | | | GCTCTTAGCGCAATGTATGTGCTGTTAT | | GGGCTGGCAACCTGGAACAAAAGT |
| | | | | | C | | C |
| NP | PT | | NM_ | 215 | TATTAAGGCCCGAGGCACCCACGTCGA | 216 | CGAAAGCCATGACCTCCGATCACTC |
| AS | | | 002518.3 | | GTGTGTTAAAGTGCTGCCGAGGTCGCTG | | GTGCGAGGATTTGTGGGAACTTCTT |
| 2 | | | | | GGTTAAATAGAGAAATGGCTACTCATTT | | GAGGACGCCGATGGCGAATGACTG |
| | | | | | AG | | G |
| NP | CI | | NM_ | 217 | AAATAGACAGAGGCGTACATGTCTGCTG | 218 | CGAAAGCCATGACCTCCGATCACTC |
| HP | | | 0011 | | AGAACCTGTATGCTCATTCCTGCCTTAC | | TGCCATGTGGCTCTGACTGTATGAA |
| 1 | | | 28179.1 | | GACTTCACTGCAATTGACGATTCAGTTA | | TGTTGCTCAGAACCTTATTACCATO |
| | | | | | A | | |
| NP | CI | | NM_ | 219 | CTGTAATCCACTGGTTAGTCTTTTAATT | 220 | CGAAAGCCATGACCTCCGATCACTC |
| HP | | | 025114.4 | | TGCCTTTTCAGTTCATCATTCTCCTCAT | | CTTTGGAGTTCTTCAATTAGACTTT |
| 6 | | | | | ACCAATGTAAAGTATAGTTAACGCCCTG | | GTTTATTATCTGTCAGGGGTTTGCC |
| | | | | | T | | |
| NP | PO | | NM_ | 221 | AAGTTTAGTTTCTAAAGCCAACCCCAGG | 222 | CGAAAGCCATGACCTCCGATCACTC |
| HS | | | 004646.3 | | ATGCACCTTGTTTCTACTCTGGCGTGAA | | AGCATTTCATTTTTGAGACGACGTT |
| 1 | | | | | CCAGATTATGTATGGACGCGCAATAGAT | | TACAATCTGCCCTGTCCTTTTGGAG |
| | | | | | A | | |
| NR | Y | | NM_ | 223 | CAGAACCCTGGATTGCAAAGAGCGCACT | 224 | CGAAAGCCATGACCTCCGATCACTC |
| K | | | 198465.1 | | TCAATGGCCTTTTGGCCCCATCCCCCAA | | GTGCACAGGAATCTCAGCTTCTTAA |
| | | | | | CAACCGCCGGATATACGTTGGGATATAA | | TTGACCTGCGCTTCAGCTCACTTTC |
| | | | | | A | | |
| OC | S | | NM_ | 225 | TGCCCCCACCCTTTGTGTTCCCAATTCC | 226 | CGAAAGCCATGACCTCCGATCACTC |
| T4 | | | 002701.4 | | TTCCTTAGTGAATGAAGAACTTCTGACA | | CATCATTGAACTTCACCTTCCCTCC |
| | | | | | CATTAGTAACGTCGGCAAGCACTTAGTC | | AACCAGTTGCCCCAAACTCCCC |
| | | | | | G | | |
| OD | Y | | NM_ | 227 | CCACGCAGGCCCTGACATCACATAGTAG | 228 | CGAAAGCCATGACCTCCGATCACTC |
| C1 | | | 0012 | | ATCGTCGGCCCCCTCTTAACTATAGCTC | | CTACTTCGGGTGGGAAGTCGGGGT |
| | | | 87189.1 | | CATTTGGACAGACGTT | | TCTGGAATTGCTGCATGAGTTG |
| OS | SC | | NM_ | 229 | CTCACTCTCCGCAGTCAGAGTTACAAGC | 230 | CGAAAGCCATGACCTCCGATCACTC |
| R1 | | | 145260.3 | | TTATATGGTCAAAGACAAAAATCGCGAT | | CTGCCAGTGCTAAGGACTTCGTTGC |
| | | | | | ACTGATGATGAAATTTTCAAAAATGGCT | | CTCTCCTCTCTCCA |
| | | | | | G | | |
| PA | UB- | | NM_ | 231 | AAATAAATGTAGACTTTTTGTGGTTCTG | 232 | CGAAAGCCATGACCTCCGATCACTC |
| X2 | CD | | 000278.3 | | TTCAGTTCCTGCCGGAGGAGGACAACAG | | CTGGTGGGCTTTTGTGTTGTTTTGT |
| | | | | | CCACTTTTTTTCCAAATTTTGCAAGAGC | | TCCTTTTTGCAAAGACCATCATATT |
| | | | | | C | | |
| PA | MET | | NM_ | 233 | AGAGGGAAGTTCTGCCATTGAAAAACCA | 234 | CGAAAGCCATGACCTCCGATCACTC |
| X8 | | | 013953.3 | | GCCGCATCTCAGATCCCTTCAGCACTAC | | TCAGGAAGACTCTCAGATGTCATGG |
| | | | | | GATCCCATGAGAACACCTGTAGATACTC | | ATTCGGAGTCGCGCTGC |
| | | | | | A | | |
| PC | DT | | NM_ | 235 | CTGCTTGAAGATGGCATCACGGCCTTCC | 236 | CGAAAGCCATGACCTCCGATCACTC |
| BD | | | 000281.2 | | AGCTCATTCCTGGTCTAGGTATCTAATT | | GCCACTCTTGTCATGAACCCAAAGG |
| 1 | | | | | CGTGGGTCGGGTACT | | CCCTGTTGAAGTCTTTGAAATGAAA |
| PD | SC | | NM_ | 237 | GAGTTAACAGTTCAAAAGTATAGCTCTT | 238 | CGAAAGCCATGACCTCCGATCACTC |
| GF | | | 0013 | | CACAGCATCTTCATTTTGAGCTCGTGGT | | AGTTGAGCCATGGTGATCATCGACC |
| RA | | | 47829.1 | | ATCCCTGGTTGAGACATTGGACTAGTGT | | AAGTCCAGAATGGATGAAGGAACTT |
| | | | | | A | | |
| PD | SC | | NM_ | 239 | CTGGTGCAGGCTCCTGAAGGCTCAGGA | 240 | CGAAAGCCATGACCTCCGATCACTC |
| GF | | | 002609.3 | | GAACAGAGGGATGCCTGAATCAATAGAA | | GTCCCAGAGTGGGTAACAGCTGAG |
| RB | | | | | CAATATCAGTTATGGCGGTG | | TAGAAGGACAGGCAGGA |
| PE | V | | NM_ | 241 | TGGACTGTGTTGCTTTTCTTGACCACTT | 242 | CGAAAGCCATGACCTCCGATCACTC |
| CA | | | 000442.3 | | TGTCAATACCTGCAGTGCTTGAGCTCTA | | GGGCATCATAAGAAATCCTGGGCTG |
| M1 | | | | | GGCCCAAAACGACCTTAATGGTCA | | GGAGAGCATTTCACATACGACTATC |
| PE | CD | | NM_ | 243 | GATGGAAAATGATGCAGCCAGCATCTCC | 244 | CGAAAGCCATGACCTCCGATCACTC |
| ND | | | 000441.1 | | GAGAACAAGCTCACAGGTGGAACTTGGA | | GTGGCATATACTTTTCCTACTGACA |
| RI | | | | | GGAGTTGATAGTGGTAAAACAACATTAG | | CTGCAATAGCATAAGCCACCACAGC |
| N | | | | | C | | |
| PIE | X | | NM_ | 245 | GCGGCCGGCGAAGCTGACCACGATGAA | 246 | CGAAAGCCATGACCTCCGATCACTC |
| ZO | | | 0011 | | CATGCCAGCACACACATAGATCCTTCTG | | GTGAGGCAGAGCAGGAAGAGGAAC |
| 1 | | | 42864.1 | | GAATTTCTTCCTTTGATTTTGCCATTTT | | ATGTAGACAATCTTGTAGACCACGA |
| | | | | | | | G |
| PK | CI | | NM_ | 247 | CGGAAGGTGTAAGAGATGGTAGGACCC | 248 | CGAAAGCCATGACCTCCGATCACTC |
| D1 | | | 0010 | | CCAGGGATGGGCGCATCTCCATGACTG | | CTCGTTCTCAGCCGTGACGATGATA |
| | | | 09944.2 | | CTTGAGCGGCTGGAGAATCTG | | TTGAAGGTGCCCACGGAG |
| PK | CI | | NM_ | 249 | ATTCAACAGCACTGCATGCTCTTTTGCT | 250 | CGAAAGCCATGACCTCCGATCACTC |
| D2 | | | 000297.3 | | TATTAGCGCTCAAAATGTCACCCTTTCG | | AAAATATAGCATTAAACGACAGGAT |
| | | | | | CCACCCATATAAACCCCACTTCGTCCTC | | TCTCTTGGTTCTCCAAGTGAGGGTT |
| | | | | | A | | |
| PK | CI | | NM_ | 251 | ATAATATCAGGCTAACACCTTCCAAACT | 252 | CGAAAGCCATGACCTCCGATCACTC |
| HD | | | 138694.3 | | AGAGCCTCGGATGGTGGCCCAGCAAGGC | | TCTGCTTGAATTGCTTGTAGCGACA |
| 1 | | | | | AGAGCAAATGTGACACTGTCTATCAGTA | | TTGATGGCACACGAGTAAGATCCAA |
| | | | | | C | | |
| PL | Y | | NM_ | 253 | GAGCGACCCAGGTAGACGATGTAGTCC | 254 | CGAAAGCCATGACCTCCGATCACTC |
| AU | | | 002658.2 | | TCCTTCTTTGGGTAATCAATGAACAAAG | | GGTTTTCCACCTCAAACTTCATCTC |
| | | | | | CAAACCTAATGAAGCCAGACACGAGATC | | CCCTTGCGTGTTGGAGTTAAGCCTT |
| | | | | | AC | | |
| PO | PO- | | NM_ | 255 | CTCAATCAGATGAAACCTCACCATGCCC | 256 | CGAAAGCCATGACCTCCGATCACTC |
| DX | S | | 005397.3 | | ACTGCTTAGGAGCCTTCTACATCTAGCC | | ACGATGGAGACCATGGCGAAAGTT |
| L | | | | | CAGATCCTACGAGATGAGCTACGTAACT | | CAACATTCCACACAGGATTCCCCCT |
| | | | | | A | | T |
| PO | LH- | | NM_ | 257 | TTGGATCCCGGGCGGCGTCATGCGCTT | 258 | CGAAAGCCATGACCTCCGATCACTC |
| U3 | DT | | 006236.1 | | CTCCTTTTCCATACGCATGACTACATTA | | CCCACCTGCGAGTAGACGTCGTCG |
| F3 | | | | | CAACGGGCCAGGAAG | | GGCGTCTGCTG |
| RB | N | | NM_ | 259 | TGGTGGCAGAATTTCCTCTGGCTGCTGT | 260 | CGAAAGCCATGACCTCCGATCACTC |
| FO | | | 0010 | | GACTTCAAGCTGCCTATTGAAGCAATCC | | GGTTCCCAGCTGGAGGGGCTGCAG |
| X3 | | | 82575.2 | | TCTCCCCAATACTTAAAAA | | ACCTGGGAAAA |
| RE | X | | NM_ | 261 | GGAGGGAAAGTCTTGGATGCACCTGGC | 262 | CGAAAGCCATGACCTCCGATCACTC |
| N1 | | | 016118.4 | | ACTCAATCCACTCGGCCCAGCTGTCTCA | | CGCATGATCCATCCTGTCTTCAGTC |
| | | | | | ATTATTGCTCAAAAACCACTATT | | AGTGCCTTCTGGAAG |
| RE | UB | | NM_ | 263 | CTGGACGTTGATGCCACTGAATGCCTGG | 264 | CGAAAGCCATGACCTCCGATCACTC |
| T | | | 020975.4 | | CAGTTTTCCACACAGACTTTCCCATCCT | | GTGACCACCCCTAGCGTGCTGCAG |
| | | | | | CTTCTTTTCTTGGTGTTGAGAAGATGCT | | TTGGCACCAGAGGAATGCAGCTTGT |
| | | | | | C | | A |
| RO | X | | NM_ | 265 | ACATACTTCACTTTGAAGCCTTGGTTTA | 266 | CGAAAGCCATGACCTCCGATCACTC |
| BO | | | 0011 | | TTAATCACCAACACCTGCCAGTCTATCG | | CAGGAAGGTATGATGCTATGCCCAC |
| 2 | | | 28929.2 | | TGGACCCAAAGTCGATACGTCCGATTAA | | GTTATTCCAAAAGTCATCTGCACAC |
| | | | | | G | | |
| SB | UB | | NM_ | 267 | AAGTTCCTTGACACCGAGGATTACCAAT | 268 | CGAAAGCCATGACCTCCGATCACTC |
| SP | | | 153225.3 | | TGCTTGCCAATGGAGAGTCTGACCGGG | | ACTGTGAACAGCTGGACAAGAACAC |
| ON | | | | | AATCGGCATTTCGCATTCTTAGGATCTA | | TGGTCTACTCGCCGAACTTTTTTCC |
| | | | | | AA | | |
| SH | UB | | NM_ | 269 | CCTGAAAGTCCCAAGGCATCAGCTGGG | 270 | CGAAAGCCATGACCTCCGATCACTC |
| RO | | | 133456.2 | | ACAGGTTTGAGGACCCGAAGCAATACTG | | GGCTAGGGCAGTATTGTGAGAGGG |
| OM | | | | | TCGTCACTCTGTATGTCCGT | | ACCTGGAGTATCATTG |
| 1 | | | | | | | |
| SIX | X | | NM_ | 271 | GGCGGAGGAGTAGGGGAGGTTCTGGAC | 272 | CGAAAGCCATGACCTCCGATCACTC |
| 1 | | | 005982.3 | | ACGGAACGGCTCCTAAGGTTGCTGATTT | | AAGGCGAAAACCGGAGTCGGAACT |
| | | | | | GGTTGTTGGAGACCCA | | TGGCGGTGGGCGGCCAAGGAAGAG |
| | | | | | | | AA |
| SIX | X | | NM_ | 273 | AGGGAAAAGGCCTCAGGAGATGGCCTA | 274 | CGAAAGCCATGACCTCCGATCACTC |
| 2 | | | 016932.4 | | GGTTCAAAGTCCCTGTATCAAGGCCTAG | | TTACTAGACGAAAAGCTGACACCTG |
| | | | | | CCTAAAGGTTCTTGCAGAGCAACAT | | CAGCTCCCACTGACCCACATGGGC |
| | | | | | | | G |
| SL | LH | | NM_ | 275 | CCATATACAACAAATCCGATATGGATCC | 276 | CGAAAGCCATGACCTCCGATCACTC |
| C1 | | | 000338.2 | | CTTTCTTGCCACGGGAAGGCTCCTCAGG | | TCTAACTAGTAAGACAGGTGGGAGG |
| 2A | | | | | TTGTTACTTGAAGGGTTCAACACGAGCT | | TTCTTTGTGAGGATTTCCAACCAAG |
| 1 | | | | | C | | |
| SL | DT | | NM_ | 277 | GTGGACAGGGATGTCAAAGCCTGGAGT | 278 | CGAAAGCCATGACCTCCGATCACTC |
| C1 | | | 0011 | | TACTGGCAGTAAAAGGTGAGCACCATTA | | CTGTGGACTAGAGGCAATCAAGTTC |
| 2A | | | 26107.1 | | GCTCGGATGCTATCAGCTTGCGCCTATT | | CAACTTATCCCACACACTCAGAGCT |
| 3 | | | | | AT | | |
| SL | PT | | NM_ | 279 | GACAGTGGAGGGCCTCTGGTTTGCTTTG | 280 | CGAAAGCCATGACCTCCGATCACTC |
| C2 | | | 021977.2 | | AGAAGGACAAATACATTTTACACTTAGG | | GGCTGGCAGCCTAACCCTCACATG |
| 2A | | | | | CTACCAAATGAATTTAAAGCCAGCTGAA | | AATTTTTCCCTACCTCTATTTAGGG |
| 3 | | | | | A | | T |
| SL | PT | | NM_ | 281 | AGCACCCTGCTAAGCTTTTGGGACCCAT | 282 | CGAAAGCCATGACCTCCGATCACTC |
| C2 | | | 0003 | | CAGAACTATATTTCCATAATTGCCTAAT | | AGGATTGATCAGTGCTCCAGTTGGT |
| A2 | | | 40.1 | | TAGCTCTAGGAAACACAACCCCGGGATT | | GGAGAAAACAGCCTAGAGATACGTT |
| | | | | | T | | |
| SL | PT | | NM_ | 283 | TCAGGAGCATCACGGCCACCATGGATGT | 284 | CGAAAGCCATGACCTCCGATCACTC |
| C3 | | | 003052.4 | | TGAAGGAGGCTTTTTTGCAATTAAACTG | | TGGATGATGAGCTTCGTGAAGGGCT |
| 4A | | | | | CCCAGGCGATCTGTTG | | CTGTGATGATCTTGAGCAGG |
| 1 | | | | | | | |
| SL | PT | | NM_ | 285 | ACAGAGATTATGGGCACAGATTCAGGAC | 286 | CGAAAGCCATGACCTCCGATCACTC |
| C4 | | | 014585.5 | | TTGTCTCCGGGACAATATTAGCCCAATG | | AGGACCAAAGACCGATTCTAGCAGC |
| 0A | | | | | CTTGCAGTATGTATCCTGATCGTGCGTG | | AATGACGCCTGCAAACAGCAGACTG |
| 1 | | | | | C | | |
| SL | DT | | NM_ | 287 | ACCTTCTTGGCACAGCAGCAGTGTGGCC | 288 | CGAAAGCCATGACCTCCGATCACTC |
| C4 | | | 017836.3 | | CACGGAGCACGATCTGTATTTTGCACCT | | ATGAAGGGTTGTATGAGGCCCCATC |
| 1A | | | | | TTCGCTATGCTGAG | | CTGGGGAGGCTGTAC |
| 3 | | | | | | | |
| SL | PT | | NM_ | 289 | TGTAGGTCAGCTCCTGACTGACCTCTAC | 290 | CGAAAGCCATGACCTCCGATCACTC |
| C6 | | | 0010 | | CACGAAGAAGAAGAGGAAGATGCTTGA | | GGAGATCTTCTGGGATTTGGGAAAT |
| A1 | | | 03841.2 | | GTTATACGGAACTTCGCAAAAGTATTCC | | TCCTCGTAGCCAGGGTCCCAGATG |
| 9 | | | | | CT | | C |
| SL | Y | | NM_ | 291 | AAACGATGATGTGCTTTTTGAATGGGGT | 292 | CGAAAGCCATGACCTCCGATCACTC |
| C8 | | | 0011 | | TATTCTTACTGTTTGCATAGGGCGTTCA | | ATCCCAAGCAACGGCAACATGGGA |
| A1 | | | 12800.1 | | ATCGCTTAGTTTCGTGGCGGGATTTGAG | | CTGAGACACATGTTAATGCAAATGG |
| | | | | | G | | A |
| SL | PT | | NM_ | 293 | CGGTGACTGCGTCGTTCAGCAGCGACT | 294 | CGAAAGCCATGACCTCCGATCACTC |
| C9 | | | 004174.2 | | CCCCGAAGACGATGATGAACAGGCCAT | | GTTGTCACCTCCCAGCGCCACGAAA |
| A3 | | | | | CCATCAACAACTGCTCCAACAGCCTTTC | | GATTCAAACACATTGTACAGAACCA |
| | | | | | CAT | | |
| SN | SC | | NM_ | 295 | GATTGGGGTCGGAGGGCTTCCTGACGA | 296 | CGAAAGCCATGACCTCCGATCACTC |
| Al1 | | | 005985.2 | | GGAAAGAGCGCGGCATAGCATTACGAC | | TAAACTCTGGATTAGAGTCCTGCAG |
| | | | | | TTTACGAGTTCGCAGAACAAAGACTT | | CTCGCTGTAGTTAGGCTTCC |
| SO | S | | NM_ | 297 | ATCCTGCCGCCGCCGATGATTGTTATTA | 298 | CGAAAGCCATGACCTCCGATCACTC |
| X2 | | | 003106.2 | | TTATTTTTTGGAAAGGCTTAAGCCGATC | | GGCAAACTGGAATCAGGATCAAAAA |
| | | | | | TTCATAACGGACAAACTGAACGGGCCAT | | AAGCGCTTCCCTCCTCCTCTGGCCG |
| | | | | | T | | |
| SO | UB | | NM_ | 299 | TTTCTCGTTGATTTCGCTGCTCCATTTA | 300 | CGAAAGCCATGACCTCCGATCACTC |
| X9 | | | 000346.2 | | GCCAAGGTTGGCCTGGCCACTGCACAAT | | ATTCTCCATCATCCTCCACGCTTGC |
| | | | | | TCTGCGGGTTAGCAGGAAGGTTAGGGAA | | TCTGAAGAGGGTTTAAAAAGTCCAG |
| | | | | | C | | |
| SP | Y | | NM_ | 301 | CTGACTCGTTTCATAACTGTCCTTCCCA | 302 | CGAAAGCCATGACCTCCGATCACTC |
| P1 | | | 000582.2 | | CGGCTGTCCCAATCAGAAGGCGCACTGG | | TTGTGGCTGTGGGTTTCAGCACTCT |
| | | | | | TTGGTTATCCAATGATCGACCTGGAGTC | | GGTCATCCAG |
| | | | | | T | | |
| ST | UB | | NM_ | 303 | GGATCTGGATACACTGAAAGCTTAGGTG | 304 | CGAAAGCCATGACCTCCGATCACTC |
| C1 | | | 003155.2 | | AGGATTTGATGAGGATTTTTTTCTTTTA | | TTCCTCTTTCCCTCTCCTGGCTTGA |
| | | | | | ACAAACACTGGCTTTTTCCATCTTGTTG | | GTGAAGATGT |
| | | | | | C | | |
| SY | PO | | NM_ | 305 | ACAACAGTTGCCGGTGGGTTCTGATTGA | 306 | CGAAAGCCATGACCTCCGATCACTC |
| NP | | | 0011 | | CATCGGTGGCTGCAAATGCACTCTATAT | | CTGAGGAATTCTGTTGGATGCTAGA |
| O | | | 09974.2 | | GGAGGGAGAGTAGCTGGAT | | AAGTGGCAGGCTCTGTGGG |
| TA | UB | | NM_ | 307 | AAGACATCCAAACTGCGTTCAGGCAGCT | 308 | CGAAAGCCATGACCTCCGATCACTC |
| CS | | | 002353.2 | | GAAACAGGCTTCTTTCCCAGTGCCGTCT | | GGCAAGCTGAAGAATAAATAGACTG |
| TD | | | | | CAGATGAGTGGGTTAATCAATCAAGTAT | | AGTTTCCGGGCAATGTCTGTCCTCA |
| 2 | | | | | G | | |
| TB | SC | | NM_ | 309 | CTACTGAAATTCCTCACCGTGTTATTTA | 310 | CGAAAGCCATGACCTCCGATCACTC |
| X1 | | | 0010 | | ACACCATACAGGCCTAGGAACTCTATGT | | GCTTTGGCCTTTGCACTATAGCTCC |
| 8 | | | 80508.1 | | CGTGCTATAATGGCGTCTGTCGTGCTCA | | CAAAAGACAGATTGCTGGAGATAAC |
| | | | | | T | | |
| TG | Y | | NM_ | 311 | CACAAAAGGCTGCACAGGTGATTACAGG | 312 | CGAAAGCCATGACCTCCGATCACTC |
| FA | | | 003236.2 | | CCAAGTAGGAAGGTCTGTGGCACCATCC | | ACACTGAATAACCCCAAGCAGACGG |
| | | | | | ACTTTCATGGAAACAATAAGAGCAGGGA | | AGTTCTTGACAGAGTTTTGAAGGCC |
| | | | | | A | | |
| TG | X | | NM_ | 313 | CCTTCTGCTCTCCTGAAGCCACCTTGCT | 314 | CGAAAGCCATGACCTCCGATCACTC |
| FB | | | 0010 | | AGATGGGAACTGAGACATGATTCTGGAA | | GCTGGAAGGGAGGCTGGGTCTTTT |
| 111 | | | 42454.1 | | TATCCTCATGCTCATAGATGTCAAATTT | | CTTATCTTCAGACTGGTCCT |
| | | | | | C | | |
| TJ | Y | | NM_ | 315 | GAGGCTTAAAACTGCCAGTGTCATTTAC | 316 | CGAAAGCCATGACCTCCGATCACTC |
| P1 | | | 175610.2 | | ATCCTTCTTGGTCTCTAAGGATCATACG | | GGGTTTGGGACCAATGATGGGAGC |
| | | | | | CCTTACGCCCCAAGGTTTAGCGTATCAA | | ACCTGAAGGTTTAGATGCTACTTCT |
| | | | | | A | | G |
| TR | PT | | NM_ | 317 | AAGCTATGTTCCTCCTTATGATTTAGTT | 318 | CGAAAGCCATGACCTCCGATCACTC |
| PS | | | 0012 | | CTGCAGCATCACTCTGATCTAGCTCAAT | | CTGCGCTTTTCAAGTCCTTCTTACT |
| 1 | | | 82902.1 | | TGCCATTGCTGGAATAGTGTCGGTA | | GCTAGAAGATGGATCTTGAACATGC |
| TU | HK | | NM_ | 319 | ACTGCTGACACCTCCCTTGAAGCTGAGA | 320 | CGAAAGCCATGACCTCCGATCACTC |
| BB | | | 178014.2 | | TGGGAAATGGACATACTTAGAACGCTCA | | ACAGACTCCTCCAGAGTAGAGCTTG |
| | | | | | TTTTGAACATACGATTGCGATTACGGAA | | GAGGGAGATTGAAAGTGGAGATAAT |
| | | | | | A | | |
| TW | SC | | NM_ | 321 | TCCTGGAAACGGTGCCGGTGCTGCAGA | 322 | CGAAAGCCATGACCTCCGATCACTC |
| IST | | | 000474.3 | | GCCCGCGACAGCGAATCTGCAACTAAC | | AAAAAGAAAGCGCCCAACGGCTGG |
| 1 | | | | | GCAAGTTACATCCTAG | | ACGCACACCCCGCCAGGCC |
| UM | LH | | NM_ | 323 | TCATAGTTTCCAGCAAACCGGAACATCT | 324 | CGAAAGCCATGACCTCCGATCACTC |
| OD | | | 0010 | | GGACGGAAAATCGGCCCTGGGACGTCG | | TTTCATTCATGGTGTCACAGAGATA |
| | | | 08389.1 | | TGTCTTAGACGACTGTGTGTGATTCTCG | | GACTTCACAGTGCAGGTAGACTAGG |
| | | | | | AG | | |
| UP | U | | NM_ | 325 | GCTCAAATGGCAATCACAAACGGTTCAG | 326 | CGAAAGCCATGACCTCCGATCACTC |
| K1 | R | | 007000.3 | | CAAGAATGGCGATGCCTGCCATCTCATG | | GACAGTAAAGTGACCGTGACAGAAC |
| A | | | | | TCCTCTGTTAATCCAGCCTGAATATGCC | | TCTCATGGCAATAGAGGCTTCCAGA |
| | | | | | A | | |
| UP | UR | | NM_ | 327 | TGCTTCAGGAAGAGGTTGGGTGTGAAAA | 328 | CGAAAGCCATGACCTCCGATCACTC |
| K1 | | | 006952.3 | | AGTCTTGTTGTGTTGCTGCTGTCAAACT | | ACTGGTCATCATTGTTTGGAGGGCT |
| B | | | | | GGAGAGAGAAGTGAAGACGATTTAACCC | | GTTGTTTTGGTACCTCTCTAGCATC |
| | | | | | A | | |
| UP | UR | | NM_ | 329 | ACGAGGTTTGTCACCTGGTATGCACTGA | 330 | CGAAAGCCATGACCTCCGATCACTC |
| K2 | | | 006760.3 | | GCCGAGTGACTGCGATTGCTGCATTCCG | | TCCCCTTCTTCACTAGGTAGGAAAT |
| | | | | | CTCAACGCTTGAGGAAGTA | | GTAGAATTTGGTTCCTGGC |
| UP | UR | | NM_ | 331 | GGGCCTCTTTGCTGTCAAACATGCAGAG | 332 | CGAAAGCCATGACCTCCGATCACTC |
| K3 | | | 0011 | | AGGCTTTTCGCCCTGGAACAACGGTTAT | | CAGGACATACAGGTAGACCTCGTG |
| | | | 67574.1 | | ATTCTCTGGCAGAAC | | GGTGCCAGTGA |
| VE | Y | | NM_ | 333 | ATTTTCCAATATTCTGGGTAGAGTACAG | 334 | CGAAAGCCATGACCTCCGATCACTC |
| GF | | | 005429.4 | | TCATGAGTTCATCTACACTGGACCGAGT | | CCTGTTCTCTGTTATGTTGCCAGCC |
| C | | | | | GCATGAGCTGTCTTTCACATGATACATC | | TCCTTTCCTTAGCTGACACTTGTAC |
| | | | | | G | | |
| VIM | SC | | NM_ | 335 | CGAATGTGCGGACTTTTTGTTAGCAAGA | 336 | CGAAAGCCATGACCTCCGATCACTC |
| | | | 003380.3 | | AACTTCTGCAGCCTTTGGAGAGCTGTGA | | GACAAGAGCGCCCCTAAGTTTTTAA |
| | | | | | CATCGCTTCTAGAGTATGAGCCGCAATG | | TAACTCGCTAAAGCCTGTCTTTGCT |
| | | | | | C | | |
| WN | UB | | XM_ | 337 | CAAAGCACAAGCCGCCTCCCATGCCTG | 338 | CGAAAGCCATGACCTCCGATCACTC |
| T1 | | | 01154 | | GCATCTGGAATTCACAAGCCTGGAGTTT | | CAGACCTTCTGTTCCTGGTGGCTTC |
| 1 | | | 5241.2 | | ATGTATTGCCAACGAGTTTGTCTTT | | CAAGTGAAGG |
| WN | UB | | NM_ | 339 | CAGCCTGGGAACTGGTCTGGAGAGGCC | 340 | CGAAAGCCATGACCTCCGATCACTC |
| T7 | | | 058238.1 | | AGCCGGCGTAGCTTTTCTGTGTCCTGTT | | TAATTTGTGTCAACATCTGTCCCCA |
| B | | | | | GAGATTATTGAGCTTCATCATGACCAGA | | CCGCCCCAGGGCCCAGCGGCAGAC |
| | | | | | AG | | C |
| WN | UB | | NM_ | 341 | CTTGGTCTCCCTTATTTCCCTCCATGTC | 342 | CGAAAGCCATGACCTCCGATCACTC |
| T9 | | | 003396.1 | | TTTCTCACTCTGCCAAAGACGCCTATCT | | CTCTCTCCCGTCCAAAATTTCAGTC |
| b | | | | | TCCAGTTTGATCGGGAAACT | | CTGCTGTGCT |
| WT1 | PC | | NM_ | 343 | TGACCTCGGGAATGTTAGACAAGATTCA | 344 | CGAAAGCCATGACCTCCGATCACTC |
| | | | 000378.3 | | CCCCCGATGCCTTGCTCTCTGACGCTCA | | AAAAGTTGCCTGGCAGAACTACATC |
| | | | | | CGTGATCTACCCTAGCTGACCGCTAATG | | CTGCTTTCCAGGTTAGCAGCCTGGC |
| | | | | | A | | |

### Statistical Analysis:

The number of biological replicates necessary to achieve power of 0.8 were estimated using G*Power using previous or preliminary data. Statistical significance was calculated by the indicated tests using GraphPad/Prism. For all experiments, N values were the sum of replicates collected over at least 3 independent experiments. Statistical details (exact value of N and what N represents, statistical test used for comparison, definition of center, dispersion and precision measures, and definition of significance) can be found in figure legends.

### Results

### Increased Scale of UB-Like Organoids

Howden et al. (2021), Shi et al. (2022) and Zeng et al. (2021) recently described methods for generating human UB from iPSC.

Here, a protocol described by Zeng et al. was used to generate UB cells because (i) it allows for direct differentiation from hiPSCs, and (ii) a relatively homogenous UB tip-like cell population is generated which facilitates scaling, digesting, and reseeding cells on-chip. Moreover, Zeng et al. reported WNT11 expression by UB organoids and UB to CD-like cell differentiation may facilitate recombination with metanephric mesenchyme organoids. However, single organoid culture with individual embedding and feeding steps in 96-well plates currently limits scalability.

Here more cells were required for chip experiments, but it was important to confirm that potential changes in paracrine signaling or growth factor depletion did not negatively affect the UB differentiation.

Thus, a study was designed to test whether it is possible to scale up UB organoid generation via a microwell (μ-well)/transwell platform compared to an established 96-well format (FIG. 13A).

First, the UB-like phenotype was validated to be sure that UB-like cells were obtained (FIG. 13B). Transcriptional profiling revealed upregulation of UB associated genes, and downregulation of stemness makers in UB organoids compared to iPSCs in both culture formats (FIG. 13B,C). Stemness markers PODXL, OCT4, SOX2, MYC and NANOG were significantly downregulated in organoids. Conversely, significant upregulation of key UB markers (e.g. RET, GFRA1, EMX2, WNT11, SOX9, WNT9b, TACSTD2, GATA3, PAX2) was observed (Howden et al., 2021; Tsujimoto et al., 2020).

Based on the previous protocol, a study was designed to evaluate gene expression at day 21 (an early time point at which the phenotype is expected to be stable) and to compare this phenotype to a later timepoint at day 35.

With few exceptions (MMP7 and SOX2), no significant changes in gene expression were observed between the established 96-well method and the scale μ-well method at day 21 or day 35 (FIG. 13D).

This suggests that there is at least a 2-week time window in which the UB phenotype is relatively stable.

Importantly, no significant differences were observed between 96-well and μ-well culture formats (FIG. 13E), indicating that the change in format did not deleteriously affect differentiation. Expression of broad UB markers such as ETV5, KRT8 and E-CAD was confirmed by immunofluorescent staining of UB organoids in all conditions. (FIG. 13E).

Generation of CD-Like Organoids from UB

A study was designed to evaluate whether CD-like organoids can be generated from UB using Zeng et al. protocol.

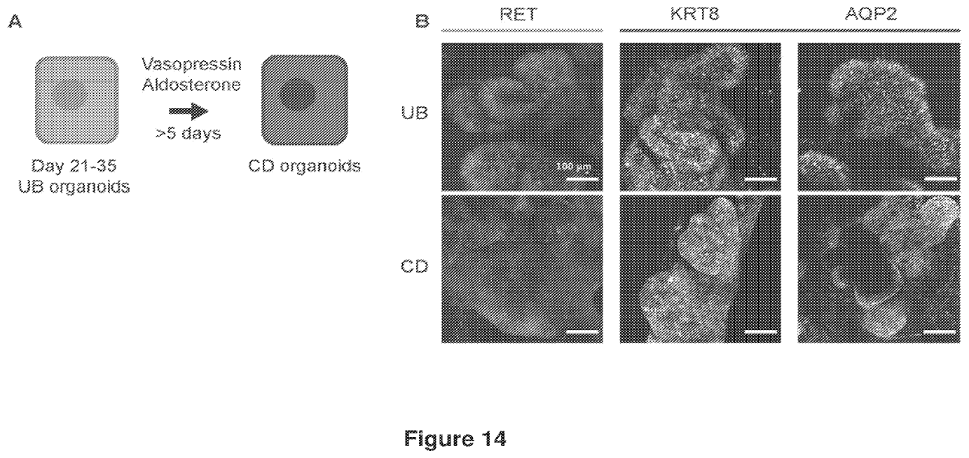

In this protocol, UB growth factors were removed and vasopressin and aldosterone were added to the culture medium for at least 5 days to induce CD differentiation (FIG. 14A).

Immunofluorescent staining of CD and UB organoids demonstrated a loss of RET and an increase in KRT8 protein expression in CD relative to UB organoids (FIG. 14B).

AQP2 expression appeared in both UB and CD.

These results indicate a differentiation of UB to CD organoids.

### Matrix Optimization for Culturing UB Monolayers

The role of ECM in UB morphologic and functional differentiation is poorly understood. Most studies of UB branching morphogenesis and differentiation to date (and our results thus far) have been performed in Matrigel or Geltrex, which is rich in basement membrane proteins such as collagen IV and laminin. However, matrix composition can affect UB morphologic differentiation.

For example, collagen I is a primary component of kidney interstitial matrix and is known to support the collecting duct through specific integrin expression (Chen et al., 2021).

A study was designed to determine which material would be optimal for chip experiments.

Initially gelbrin, which is a mixture of gelatin and fibrin that was previously used on-chip kidney models (Aceves et al., 2022; Rein et al., 2020) was tested, as well as basement membrane matrix (BsM: Matrigel or Geltrex), collagen I, and a mixture of BsM and collagen I.

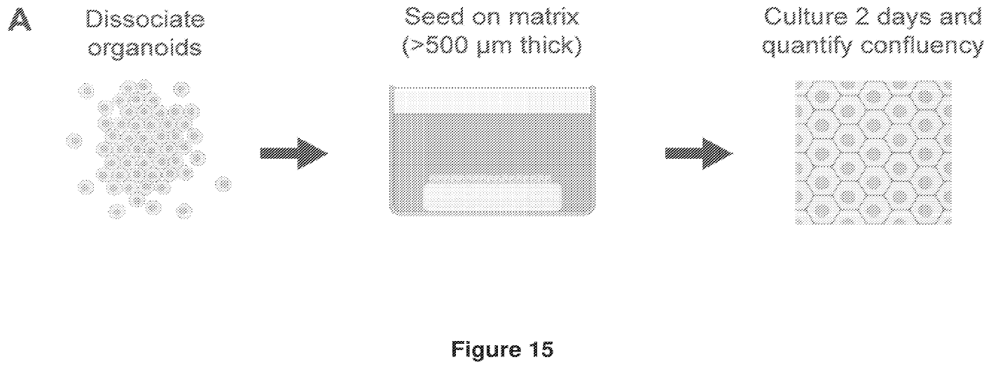

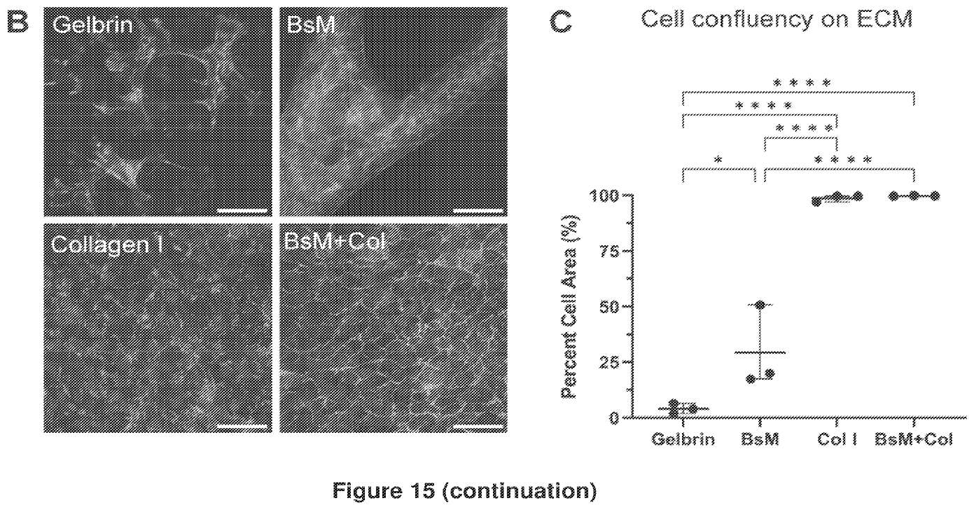

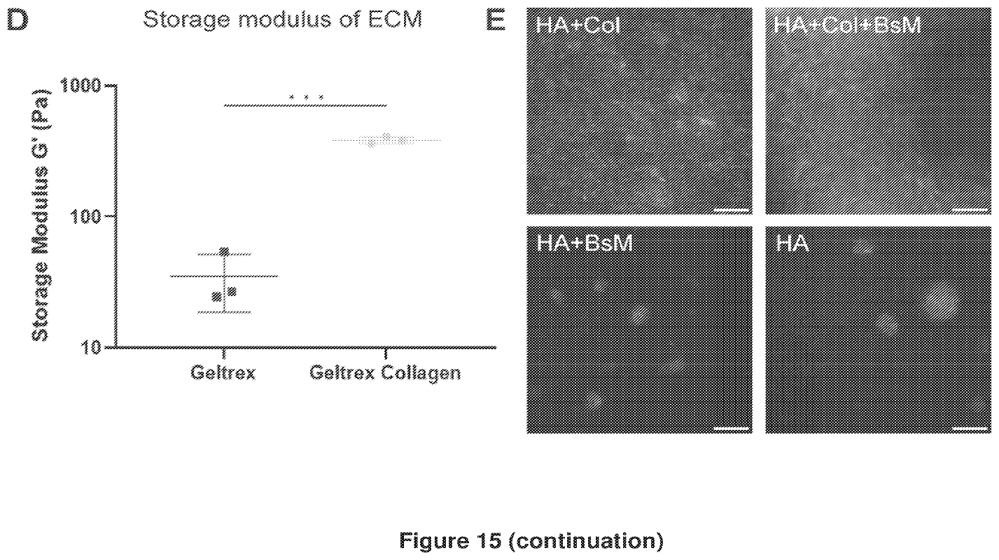

Toward the goal of seeding cells on precast gels, the potential for extracellular matrix substrates (ECM) to support initial adhesion and potential monolayer formation was tested. UB organoids (day 21-35) were dissociated and seeding them onto precast hydrogels that were >500 μm thick to ensure no effect from the glass substrate below was tested. After 2 days, confluency was imaged and quantified (FIG. 15A).

Gelbrin did not promote strong attachment or confluent monolayer formation (FIG. 15B,C). Only a few single cells remained attached and spread on gelbrin alone after 2 days.

BsM alone also did not form strong attachment; in this case cells aggregated into chords or spheroids on the surface of the BsM gels.

Both collagen I and BsM-collagen I supported attachment and the formation of a confluent monolayer, but the addition of BsM improved the epithelial morphology as evidenced by the cobblestone appearance with lateral actin localization (FIG. 15B,C).

However, both BsM and BsM-collagen I formed highly compliant matrices with storage modulus of ˜30 Pa and ˜300 Pa, respectively.

In the initial attempts to put these matrices on chip, it was found that channels made from BsM-collagen I would undergo dramatic strain changes when perfused, leading to channel collapse. It was hypothesized that incorporating an interpenetrating covalently crosslinked network could increase the hydrogel modulus and prevent channel collapse. Methacrylated hyaluronic was selected given i) it is widely used in biofabrication, ii) it forms a highly elastic nanoporous network that can still be degraded by cells, and iii) hyaluronic acid affects UB branching structure depending on the molecular weight and concentration in which it is added to Matrigel. (Rosines et al., 2007). The addition of hyaluronic acid increased storage modulus and prevented channel collapse.

Interestingly, it was found that HA-collagen I alone best supported confluent monolayer formation over HA-collagen 1-geltrex (FIG. 15E).

It is possible that the combination of hyaluronic acid and geltrex lead to steric hindrince of key collagen I cell-binding sites, preventing cell attachment. Nevertheless, HA-collagen I was used for the described chip experiments. The HA-collagen I could also be coated with basement membrane matrix and/or polylysine to further reinforce cell attachment.

### Fabrication of UB-On-Chip

A custom, perfusable chip was fabricated, in which the ECM surrounding the cast channel was directly bathed in medium which was exposed directly to air (FIG. 13A,B).

To fabricate this chip, first the various chip components were assembled (FIG. 17A) and ECM was added to an inner reservoir around a pin and crosslinked. The pin was then removed forming an open channel that can be seeded with cells (FIG. 17B). This design allowed for easy access to the ECM and medium bathing the ECM. After seeding, cells formed confluent monolayers along channel walls within 3-7 days (FIG. 16C,D,E). Perfused epithelialized channels retained FITC-inulin after 1 hour relative to empty channels, indicating barrier function (FIG. 16F,G). Furthermore, UB cells within the channels showed epithelial morphology with lateral CDH1 expression, apical BK-alpha expression, primary cilia, and NaK⁺-ATPase.

### SUMMARY

Here, a perfusable model of human, stem cell-derived ureteric bud model was developed.

To generate sufficient numbers of cells, the production was scaled up by adapting a microwell/transwell format without impacting phenotype. Then, 3D perfusable channel embedded in an optimized extracellular matrix was developed, where the ureteric bud cells formed a confluent monolayer and maintained their marker expression and morphology. The UB organoids were differentiated into CD-like organoids, which could also be seeded on chip or differentiated directly on chip.

This model has the potential to advance research in understanding kidney function, disease phenotypes, and drug interactions as well as for direct embedding within functional tissue.

### REFERENCES

Aceves, J. O., Heja, S., Kobayashi, K., Robinson, S. S., Miyoshi, T., Matsumoto, T., Schäffers, O. J. M., Morizane, R., and Lewis, J. A. (2022). 3D proximal tubule-on-chip model derived from kidney organoids with improved drug uptake. Sci. Rep. 12, 1-14.
Catena, J. R., Dalrymple, N.C., and Siegel, C. L. (2007). Segmental disorders of the nephron: histopathological and imaging perspective. Br. J. Radiol. 80, 593-602.
Chen, D., Roberts, R., Pohl, M., Nigam, S., Kreidberg, J., Wang, Z., Heino, J., Ivaska, J., Coffa, S., Harris, R. C., et al. (2021). Differential expression of collagen- and laminin-binding integrins mediates ureteric bud and inner medullary collecting duct cell tubulogenesis. 37232, 602-611.
Fleming, G. M. (2011). Renal replacement therapy review: Past, present and future. Organogenesis 7, 2-12.
Gaston, R. S., Cecka, J. M., Kasiske, B. L., Fieberg, A. M., Leduc, R., Cosio, F. C., Gourishankar, S., Grande, J., Halloran, P., Hunsicker, L., et al. (2010). Evidence for antibody-mediated injury as a Major determinant of late kidney allograft failure. Transplantation 90, 68-74.
Howden, S. E., Wilson, S. B., Groenewegen, E., Jain, S., Spence, J. R., Little, M. H., Howden, S. E., Wilson, S. B., Groenewegen, E., Starks, L., et al. (2021). Plasticity of distal nephron epithelia from human kidney organoids enables the induction of ureteric tip and stalk. Cell Stem Cell 28, 1-14.
Jang, K. J., and Suh, K. Y. (2010). A multi-layer microfluidic device for efficient culture and analysis of renal tubular cells. Lab Chip 10, 36-42.
Jansen, J., Fedecostante, M., Wilmer, M. J., van den Heuvel, L. P., Hoenderop, J. G., and Masereeuw, R. (2014). Biotechnological challenges of bioartificial kidney engineering. Biotechnol. Adv. 32, 1317-1327.
Knight, A. (2007). Systematic reviews of animal experiments demonstrate poor human clinical and toxicological utility. Altern. Lab. Anim. 35, 641-659.
Little, M. H., and Combes, A. N. (2019). Kidney organoids: accurate models or fortunate accidents. Genes Dev. 33, 1319-1345.
Mae, S., Ryosaka, M., Sakamoto, S., Matsuse, K., Nozaki, A., Igami, M., Kabai, R., Watanabe, A., and Osafune, K. (2020a). Expansion of Human iPSC-Derived Ureteric Bud Organoids with Repeated Branching Potential. Cell Rep. 32, 107963.
Mae, S., Ryosaka, M., Sakamoto, S., Matsuse, K., Nozaki, A., Igami, M., Kabai, R., Watanabe, A., Osafune, K., Correspondence, K. O., et al. (2020b). Expansion of Human iPSC-Derived Ureteric Bud Organoids with Repeated Branching Potential. CellReports 32, 107963.
McCormick, F., Held, P. J., and Chertow, G. M. (2018). The terrible toll of the kidney shortage. J. Am. Soc. Nephrol. 29, 2775-2776.
Mounier, F., Foidart, J.-M., and Gubler, M.-C. (1986). Distribution of Extracellular Matrix Glycoproteins during Normal Development of Human Kidney. States-Canadian Div. Int. Acad. Pathol. 54, 394-401.
Rein, J. L., Heja, S., Flores, D., Carrisoza-Gaytan, R., Lin, N.Y.C., Homan, K. A., Lewis, J. A., and Satlin, L. M. (2020). Effect of luminal flow on doming of mpkCCD cells in a 3D perfusable kidney cortical collecting duct model. Am. J. Physiol.-Cell Physiol. 318, C136-C147.
Rosines, E., Schmidt, H. J., and Nigam, S. K. (2007). The effect of hyaluronic acid size and concentration on branching morphogenesis and tubule differentiation in developing kidney culture systems: Potential applications to engineering of renal tissues. Biomaterials 28, 4806-4817.
Sellarés, J., De Freitas, D. G., Mengel, M., Reeve, J., Einecke, G., Sis, B., Hidalgo, L. G., Famulski, K., Matas, A., and Halloran, P. F. (2012). Understanding the causes of kidney transplant failure: The dominant role of antibody-mediated rejection and nonadherence. Am. J. Transplant. 12, 388-399.
Shi, M., McCracken, K. W., Patel, A. B., Zhang, W., Ester, L., Valerius, M. T., and Bonventre, J. V. (2022). Human ureteric bud organoids recapitulate branching morphogenesis and differentiate into functional collecting duct cell types. Nat. Biotechnol. 41, 252-261.
Skylar-Scott, M. A., Uzel, S. G. M., Nam, L. L., Ahrens, J. H., Truby, R. L., Damaraju, S., and Lewis, J. A. (2019). Biomanufacturing of organ-specific tissues with high cellular density and embedded vascular channels. Sci. Adv. 5, eaaw2459.
Sun, Y., Chen, C. S., and Fu, J. (2012). Forcing Stem Cells to Behave: A Biophysical Perspective of the Cellular Microenvironment. Annu. Rev. Biophys. 41, 519-542.
Takasato, M., Er, P. X., Chiu, H. S., Maier, B., Baillie, G. J., Ferguson, C., Parton, R. G., Wolvetang, E. J., Roost, M. S., De Sousa Lopes, S. M. C., et al. (2015). Kidney organoids from human iPS cells contain multiple lineages and model human nephrogenesis. Nature 526, 564-568.
Tsujimoto, H., Kasahara, T., Sueta, S., Araoka, T., Sakamoto, S., and Okada, C. (2020). Human Kidney Lineages from Pluripotent Stem Cells A Modular Differentiation System Maps Multiple Human Kidney Lineages from Pluripotent Stem Cells. CellReports 31, 107476.
Wagner, C., Devuyst, O., Bourgeois, S., and Mohebbi, N. (2009). Regulated acid-base transport in the collecting duct. 458.
Zeng, Z., Huang, B., Parvez, R. K. K., Li, Y., Chen, J., Vonk, A. C. C., Thornton, M. E. E., Patel, T., Rutledge, E. A. A., Kim, A. D. D., et al. (2021). Generation of patterned kidney organoids that recapitulate the adult kidney collecting duct system from expandable ureteric bud progenitors. Nat. Commun. 12, 3614.

1. A perfusable 3D tubule-on-chip model comprising:
organoid-derived cells; and
a multifluidic platform comprising at least one individually addressable chip,
wherein the chip comprises a first channel consisting of one patent lumen circumscribed by the organoid-derived cells,
wherein the first channel is embedded within an extracellular matrix (ECM).

2. The perfusable 3D tubule-on-chip model of claim 1, wherein:
the organoid-derived cells are organoid-derived proximal tubule epithelial cells (OPTECs) isolated from kidney organoids derived from human pluripotent stem cells (hPSCs); or
the organoid-derived cells are ureteric bud (UB) cells isolated from UB organoids derived from hiPSCs.

3. The perfusable 3D tubule-on-chip model of claim 1, wherein the chip further comprises a second channel, the second channel being empty (non-seeded),
wherein the first channel and the second channel are co-localized on the chip; and
wherein the second channel is embedded within the ECM.

4. The perfusable 3D tubule-on-chip model of claim 1, wherein the multifluidic platform comprises at least two individually addressable chips.

5. The perfusable 3D tubule-on-chip model of claim 1, wherein the multifluidic platform comprises 6 to 10 individually addressable chips.

6. The perfusable 3D tubule-on-chip model of claim 1, wherein the ECM comprises;
at least one of gelatin and fibrinogen; and/or
20 mg/mL fibrinogen.

7. (canceled)

8. The perfusable 3D tubule-on-chip model of claim 1, wherein the second channel is seeded with endothelial cells thereby creating a vascularized 3D tubule-on-chip model.

9. A perfusable 3D proximal tubule-on-chip model comprising:
organoid-derived proximal tubule epithelial cells (OPTECs); and
a multifluidic platform comprising at least one individually addressable chip,
wherein the chip comprises a first channel consisting of one patent lumen circumscribed by the OPTECs,
wherein the first channel is embedded within an extracellular matrix (ECM).

10. The perfusable 3D proximal tubule-on-chip model of claim 9, wherein the chip further comprises a second channel, the second channel being empty (non-seeded),
wherein the first channel and the second channel are co-localized on the chip; and
wherein the second channel is embedded within the ECM.

11. The perfusable 3D proximal tubule-on-chip model of claim 9, wherein the OPTECs are isolated from kidney organoids derived from human pluripotent stem cells (hPSCs).

12. The perfusable 3D proximal tubule-on-chip model of claim 9, wherein the multifluidic platform comprises at least two individually addressable chips.

13. The perfusable 3D proximal tubule-on-chip model of claim 9, wherein the multifluidic platform comprises 6 to 10 individually addressable chips.

14. The perfusable 3D proximal tubule-on-chip model of claim 9, wherein the ECM comprises;
at least one of gelatin and fibrinogen; and/or
20 mg/mL fibrinogen.

15. (canceled)

16. The perfusable 3D proximal tubule-on-chip model of claim 9, wherein the OPTECs exhibit:
at least 1.5-fold higher drug transporter expression, as compared to an immortalized proximal tubule epithelial cell line; and/or
at least 2-fold higher drug uptake, as compared to an immortalized proximal tubule epithelial cell line; and/or
a higher expression of basolateral drug transporters OCT2, OAT1, and OAT3, as compared to an immortalized proximal tubule epithelial cell line.

17. (canceled)

18. The perfusable 3D proximal tubule-on-chip model of claim 9, wherein:
the first channel exhibits a higher cell death response to known nephrotoxins, cisplatin and aristolochic acid, compared to an immortalized proximal tubule epithelial cell line; and/or
the second channel is seeded with endothelial cells thereby creating a vascularized OPTEC-on-chip model.

19. (canceled)

20. The perfusable 3D tube-on-chip model of claim 1, wherein:
the organoid derived cells are organoid-derived ureteric bud (UB) cells; and
the chip comprises a first channel consisting of one patent lumen circumscribed by the organoid-derived UB cells.

21.-27. (canceled)

28. A method of producing a perfusable 3D kidney-on-chip model comprising:
isolating organoid-derived cells from an organoid derived from human pluripotent stem cells (hPSCs);
seeding the isolated organoid-derived cells onto a multifluidic platform comprising at least one individually addressable chip,
wherein the chip contains a first channel consisting of one patent lumen, wherein the organoid-derived cells are seeded within the first channel and circumscribe the first channel.

29. The method of claim 28, wherein the organoid derived cells are:
organoid-derived proximal tubule epithelial cells (OPTECs) from a kidney organoid derived from human pluripotent stem cells (hPSCs); or
ureteric bud (UB) cells isolated from UB organoids derived from hiPSCs.

30. The method of claim 28, wherein the chip further comprises a second channel, the second channel being empty (non-seeded),
wherein the first channel and the second channel are co-localized on the chip; and
wherein the first and the second channels are embedded within the ECM.

31. The method of claim 30, further comprises seeding the second channel with endothelial cells thereby creating a vascularized 3D kidney-on-chip model.

32. The method of claim 28, wherein the step of isolating the organoid-derived cells is by magnetic-activated cell sorting.

33. The method of claim 29, wherein the isolated OPTECs are LTL+ OPTECs.

34. The method of claim 29, further comprising expanding the organoid-derived cells in 2D culture.

35. The method of claim 29, further comprising:
differentiating hPSCs into nephron progenitor cells;
producing kidney organoids from the nephron progenitor cells; and maturing the kidney organoids under static culture conditions.

36. The method of claim 29, wherein the first channel is coated with laminin-5 1 1.

37. The method of claim 29, wherein the chip is produced by:
encapsulating a first channel template and/or a second channel template within an ECM solution cast into the chip;
enzymatically cross-linking the ECM solution;
removing the first channel template, thereby forming the first channel, where the first channel can be seeded with organoid-derived cells and/or removing the second channel template, thereby forming the second channel.

38. (canceled)

39. The method of claim 29, wherein a minimum seeding density of organoid-derived cells is 10 million cells/mL.

40. The method of claim 29, wherein the ECM solution is a gelatin-fibrinogen solution.

41.-44. (canceled)

45. A perfusable 3D proximal tubule-on-chip model of claim 9, wherein:
the first channel is an OPTEC tubule;
wherein the OPTEC tubule is embedded within an extracellular matrix (ECM); and/or
wherein the OPTEC tubule exhibits:
at least 1.5-fold higher drug transporter expression, as compared to a tubule with an immortalized proximal tubule epithelial cell line, and/or
at least 2-fold higher drug uptake, as compared to a tubule with an immortalized proximal tubule epithelial cell line.

46. The perfusable 3D proximal tubule-on-chip model of claim 45, further comprising an unseeded tubule,
wherein the OPTEC tubule and the unseeded tubule are co-localized on the chip; and wherein the unseeded tubule is embedded within the ECM.

47.-54. (canceled)
